(19) United States
(12) Patent Application Publication
(10) Pub. No.: US 2024/0263240 A1
(43) Pub. Date: Aug. 8, 2024

(54) CD274 MUTATIONS FOR CANCER TREATMENT

(71) Applicant: Foundation Medicine, Inc., Cambridge, MA (US)

(72) Inventors: Richard Sheng Poe HUANG, Cary, NC (US); Brennan DECKER, Winchester, MA (US); Jeffrey ROSS, Lebanon Springs, NY (US)

(73) Assignee: Foundation Medicine, Inc., Cambridge, MA (US)

(21) Appl. No.: 18/290,138

(22) PCT Filed: May 13, 2022
(86) PCT No.: PCT/US2022/029313

§ 371 Date: Nov. 9, 2023

Related U.S. Application Data
(60) Provisional application No. 63/188,719, filed on May 14, 2021.

(51) Int. Cl. C12Q 1/6886; A61K 45/06

(52) U.S. Cl. CPC C12Q 1/6886; A61K 45/06; C12Q 2600/106; C12Q 2600/156

(57) ABSTRACT

Provided herein are methods related to detecting CD274 gene mutations, as well as methods of treatment, uses, and kits related thereto. Detection of CD274 gene mutations can be used to identify individuals that may or may not benefit from an anti-cancer therapy.

### CROSS REFERENCE TO RELATED APPLICATIONS

This application claims the priority benefit of U.S. Provisional Application No. 63/188,719, filed May 14, 2021, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 197102006240SEQLIST.TXT, date recorded: May 12, 2022, size: 7,188 bytes).

### FIELD

Provided herein are methods related to detecting mutations in a cluster of differentiation 274 (CD274) gene, as well as methods of diagnosis/treatment, uses, and kits related thereto.

### BACKGROUND

The programmed death-ligand 1 (PD-L1) protein is encoded by the approximately 17.6 kilobase CD274 gene located on chromosome 9p24.1 (Fabrizio et al., Ther Adv Med Oncol (2018) 10:1758835918815598-). The CD274 Matched Annotation from the U.S. National Center for Biotechnology Information (NCBI) and the European Bioinformatics Institute (EMBL-EBI) (Matched Annotation from NCBI and EMBL-EBI: MANE) transcript (ENST00000381577.4) encodes for a type 1 transmembrane protein that is 290 amino acids long and has immunoglobulin V-like and C-like domains.

Immune checkpoint inhibitors (ICPI) that block the PD-L1 and programmed cell death protein 1 (PD-L1/PD-1) axis have shown great clinical utility in a wide variety of solid tumors and hematologic malignancies (Li et al., Int J Mol Sci. (2016) 17(7); Schachter et al., Lancet (2017) 390(10105):1853-62; Pai-Scherf et al., Oncologist (2017) 22(11):1392-9; and Schmid et al., N Engl J Med (2018) 379(22):2108-21).

Multiple companion diagnostics for ICPI have been developed and subsequently approved by the United States Food and Drug Administration (FDA) (FDA: List of Cleared or Approved Companion Diagnostic Devices (In Vitro and Imaging Tools), available from: www.fda.gov/medical-devices/vitro-diagnostics/list-cleared-or-approved-companion-diagnostic-devices-vitro-and-imaging-tools; Gjoerup et al., Aaps J (2020) 22(6):132). A frequent ICPI companion diagnostic utilized for multiple tumor types is PD-L1 immunohistochemistry (IHC), which can detect PD-L1 protein expression and over-expression on tumor cells and tumor infiltrating immune cells. Clinical trials have shown that in specific tumor types, a certain level of PD-L1 protein expression is necessary in the tumor microenvironment for a PD-L1/PD-1 inhibitor to be efficacious (Schmid et al., N Engl J Med (2018) 379(22):2108-21; Chung et al., Journal of Clinical Oncology: Official Journal of the American Society of Clinical Oncology (2019) 37(17):1470-8; and Hellmann et al., N Engl J Med (2019) 381(21):2020-31).

Currently, in large public genomic databases like the Catalogue Of Somatic Mutations In Cancer (COSMIC), only 229 CD274 non-amplification short variant (SV) mutated samples have been reported (Tate J G et al., Nucleic Acids Res (2019) 47(D1):D941-d7; COSMIC v92, released 27 Aug. 2020 cancer.sanger.ac.uk2020; cancer.sanger.ac.uk/cosmic). Studies have examined PD-L1 protein expression in a variety of tumor types. However, limited data on CD274 SV mutations or their potential effects exists (Huang et al., Mod Pathol (2020) 34:252-263; O'Malley et al., Mod Pathol (2019) 32(7):929-42).

Thus, there is a need in the art for characterizing the landscape of CD274 mutations in cancer and evaluating their effects, and for developing methods of identifying and evaluating patients with cancer having such CD274 mutations. Such CD274 mutations can be an effective approach to develop compositions, methods and assays for evaluating and treating cancer.

All references cited herein, including patent applications and publications, are incorporated by reference in their entirety.

### SUMMARY

In some aspects, provided herein is a method of identifying an individual having cancer who may benefit from a treatment comprising an anti-cancer therapy, the method comprising detecting one or more mutations in a CD274 gene in a sample from the individual, wherein the presence of the one or more mutations in the CD274 gene in the sample identifies the individual as one who may or may not benefit from the anti-cancer therapy.

In some aspects, provided herein is a method of detecting the presence or absence of a cancer in an individual, the method comprising: (a) detecting the presence or absence of a cancer in a sample from the individual; and (b) detecting the presence or absence of one or more mutations in a CD274 gene in the sample.

In some aspects, provided herein is a method of selecting a therapy for an individual having cancer, the method comprising detecting one or more mutations in a CD274 gene in a sample from the individual, wherein the presence of the one or more mutations in the CD274 gene in the sample identifies the individual as one who may benefit from a treatment comprising an anti-cancer therapy.

In some embodiments of any of the aspects provided herein, the presence of the one or more mutations in the CD274 gene in the sample identifies the individual as one who may have a cancer that is resistant to one or more immune checkpoint inhibitors.

In some aspects, provided herein is a method of identifying one or more treatment options for an individual having cancer, the method comprising: (a) detecting one or more mutations in a CD274 gene in a sample from the individual; and (b) generating a report comprising one or more treatment options identified for the individual based at least in part on the presence of the one or more mutations in the CD274 gene in the sample, wherein the one or more treatment options comprise an anti-cancer therapy.

In some aspects, provided herein is a method of identifying one or more treatment options for an individual having cancer, the method comprising: (a) acquiring knowledge of one or more mutations in a CD274 gene in a sample from the individual; and (b) generating a report comprising one or more treatment options identified for the individual based at least in part on said knowledge, wherein the one or more treatment options comprise an anti-cancer therapy.

In some embodiments of any of the aspects provided herein, the report identifies the individual as one who may have a cancer that is resistant to one or more immune checkpoint inhibitors.

In some aspects, provided herein is a method of selecting or not selecting a treatment for an individual having cancer, comprising acquiring knowledge of one or more mutations in a CD274 gene in a sample from an individual having cancer, wherein responsive to the acquisition of said knowledge: (i) the individual is classified as a candidate to receive treatment with an anti-cancer therapy, or the individual is not classified as a candidate to receive treatment with an anti-cancer therapy; and/or (ii) the individual is identified as likely to respond to a treatment that comprises an anti-cancer therapy, or the individual is identified as unlikely to respond to a treatment that comprises an anti-cancer therapy. In some embodiments, responsive to the acquisition of said knowledge: (i) the individual is classified as having a cancer that is resistant to one or more immune checkpoint inhibitors; and/or (ii) the individual is identified as unlikely to respond to a treatment that comprises one or more immune checkpoint inhibitors.

In some aspects, provided herein is a method of predicting survival of an individual having cancer, comprising acquiring knowledge of one or more mutations in a CD274 gene in a sample from the individual, wherein responsive to the acquisition of said knowledge, the individual is predicted to have shorter survival when treated with one or more immune checkpoint inhibitors, as compared to survival of an individual whose cancer does not comprise the one or more mutations in a CD274 gene.

In some aspects, provided herein is a method of predicting survival of an individual having a cancer treated with one or more immune checkpoint inhibitors, the method comprising acquiring knowledge of one or more mutations in a CD274 gene in a sample from the individual, wherein responsive to the acquisition of said knowledge, the individual is predicted to have shorter survival after treatment with the one or more immune checkpoint inhibitors, as compared to an individual whose cancer does not exhibit one or more mutations in a CD274 gene.

In some aspects, provided herein is a method of treating or delaying progression of cancer, comprising: (a) acquiring knowledge of one or more mutations in a CD274 gene in a sample from an individual; and (b) responsive to said knowledge, administering to the individual an effective amount of a treatment that comprises an anti-cancer therapy.

In some aspects, provided herein is a method of treating or delaying progression of cancer, comprising, responsive to acquiring knowledge of one or more mutations in a CD274 gene in a sample from an individual, administering to the individual an effective amount of a treatment that comprises an anti-cancer therapy.

In some aspects, provided herein is a method of monitoring an individual having cancer, comprising acquiring knowledge of one or more mutations in a CD274 gene in a sample from the individual, wherein responsive to the acquisition of said knowledge, the individual is predicted to have increased risk of cancer resistance to one or more immune checkpoint inhibitors, as compared to an individual whose cancer does not comprise one or more mutations in a CD274 gene.

In some aspects, provided herein is a method of evaluating an individual having cancer, comprising acquiring knowledge of one or more mutations in a CD274 gene in a sample from the individual, wherein responsive to the acquisition of said knowledge, the individual is predicted to have increased risk of cancer that is resistant to one or more immune checkpoint inhibitors, as compared to an individual whose cancer does not comprise the one or more mutations in a CD274 gene.

In some aspects, provided herein is a method of screening an individual having cancer, comprising acquiring knowledge of one or more mutations in a CD274 gene in a sample from the individual, wherein responsive to the acquisition of said knowledge, the individual is predicted to have increased risk of cancer that is resistant to one or more immune checkpoint inhibitors, as compared to an individual whose cancer does not comprise the one or more mutations in a CD274 gene.

In some aspects, provided herein is a method of treating or delaying progression of cancer, comprising: (a) detecting one or more mutations in a CD274 gene in a sample from an individual; and (b) administering to the individual an effective amount of a treatment that comprises an anti-cancer therapy.

In some aspects, provided herein is a method of diagnosing/assessing one or more mutations in a CD274 gene in a cancer in an individual, the method comprising: (a) detecting one or more mutations in a CD274 gene in a sample from the individual; and (b) providing an assessment of the one or more mutations in a CD274 gene.

In some aspects, provided herein is a method of diagnosing an immune checkpoint inhibitor-resistant cancer in an individual, the method comprising: (a) detecting one or more mutations in a CD274 gene in a sample from the individual; and (b) providing a diagnosis of an immune checkpoint inhibitor-resistant cancer in the individual.

In some aspects, provided herein is a method of detecting one or more mutations in a CD274 gene, the method comprising detecting the one or more mutations in a CD274 gene in a sample from an individual having a cancer.

In some aspects, provided herein is a method of detecting one or more mutations in a CD274 gene, the method comprising: (a) providing a plurality of nucleic acids obtained from a sample from an individual, wherein the plurality of nucleic acids comprises nucleic acids encoding a CD274 gene; (b) optionally, ligating one or more adaptors onto one or more nucleic acids from the plurality of nucleic acids; (c) optionally, amplifying nucleic acids from the plurality of nucleic acids; (d) optionally, capturing a plurality of nucleic acids corresponding to the CD274 gene; (e) sequencing, by a sequencer, the plurality of nucleic acids to obtain a plurality of sequence reads corresponding to the CD274 gene; (f) analyzing the plurality of sequence reads; and (g) based on the analysis, detecting one or more mutations in a CD274 gene. In some embodiments, the plurality of nucleic acids corresponding to the CD274 gene is captured from the amplified nucleic acids by hybridization with a bait molecule.

In some aspects, provided herein is a method of detecting one or more mutations in a CD274 gene, the method comprising: (a) providing a sample from an individual having a cancer, wherein the sample comprises one or more nucleic acids; (b) preparing a nucleic acid sequencing library from the one or more nucleic acids in the sample; (c) amplifying said library using a polymerase chain reaction (PCR); (d) selectively enriching for one or more nucleic acids comprising CD274 nucleotide sequences in said library to produce an enriched sample; (e) sequencing the enriched sample, thereby producing a plurality of sequencing reads; (f) analyzing the plurality of sequencing reads for the presence of one or more mutations in a CD274 gene; (g) detecting, based on the analyzing step, one or more mutations in a CD274 gene in the sample from the individual.

In some aspects, provided herein is a method of treating or delaying progression of cancer, comprising administering to an individual having cancer an effective amount of an anti-cancer therapy, wherein the cancer comprises one or more mutations in a CD274 gene.

In some embodiments of any of the aspects or embodiments provided herein, the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA).

In some embodiments of any of the aspects or embodiments provided herein, the one or more mutations in a CD274 gene comprise one or more of a missense mutation, a truncation, a nonsense mutation, a splice site mutation, an insertion/deletion, and any combination thereof. In some embodiments, the one or more mutations in a CD274 gene comprise two or more missense mutations. In some embodiments, the one or more mutations in a CD274 gene comprise one or more missense mutations and a truncation. In some embodiments, the one or more mutations in a CD274 gene comprise one or more mutations listed in Tables 1-4 and 6. In some embodiments, the one or more mutations in a CD274 gene further comprise a CD274 gene deletion, wherein the CD274 gene is a deletion of a portion of the CD274 gene. In some embodiments, the one or more mutations in a CD274 gene further comprise a CD274 genomic rearrangement or a CD274 gene fusion. In some embodiments, the one or more mutations in a CD274 gene are somatic mutations or germline mutations. In some embodiments, the one or more mutations in a CD274 gene are clonal mutations. In some embodiments, the one or more mutations in a CD274 gene are sub-clonal mutations. In some embodiments, the one or more mutations in a CD274 gene further comprise a CD274 gene amplification.

In some embodiments of any of the aspects or embodiments provided herein, the one or more mutations in a CD274 gene result in: (a) low expression of a PD-L1 protein in the cancer, (b) no expression of a PD-L1 protein in the cancer, or (c) high expression of a PD-L1 protein in the cancer. In some embodiments, PD-L1 protein expression is assessed using an immunohistochemistry assay in sample obtained from the individual. In some embodiments, PD-L1 protein expression is assessed in tumor cells. In some embodiments, a low expression of a PD-L1 protein in the cancer is assessed based on a tumor proportion score (TPS) of between 1% and 49%. In some embodiments, the one or more mutations in a CD274 gene comprise one or more mutations listed in Table 2. In some embodiments, no expression of a PD-L1 protein in the cancer is assessed based on a TPS of less than 1%. In some embodiments, the one or more mutations in a CD274 gene comprise one or more mutations listed in Table 3. In some embodiments, high expression of a PD-L1 protein in the cancer is assessed based on a TPS of 50% or greater. In some embodiments, the one or more mutations in a CD274 gene comprise one or more mutations listed in Table 4. In some embodiments, the one or more mutations in a CD274 gene comprise one or more missense mutations, optionally wherein the one or more mutations are clonal or sub-clonal mutations. In some embodiments, the one or more mutations in a CD274 gene comprise a truncating mutation, optionally wherein the truncating mutation is a clonal or sub-clonal mutation.

In some embodiments of any of the aspects or embodiments provided herein, the one or more mutations in a CD274 gene reduce the interaction between a PD-L1 polypeptide encoded by the CD274 gene and a PD-1 receptor; and/or the one or more mutations in a CD274 gene reduce the activity of a PD-L1 polypeptide encoded by the CD274 gene.

In some embodiments of any of the aspects or embodiments provided herein, the one or more mutations in a CD274 gene result in an immune checkpoint inhibitor resistant cancer. In some embodiments of any of the aspects or embodiments provided herein, the one or more mutations in a CD274 gene are associated with an immune checkpoint inhibitor resistant cancer. In some embodiments of any of the aspects or embodiments provided herein, the one or more mutations in a CD274 gene occur in an immune checkpoint inhibitor resistant cancer.

In some embodiments of any of the aspects or embodiments provided herein, the anti-cancer therapy is a therapy other than an immune checkpoint inhibitor. In some embodiments, the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA).

In some embodiments of any of the aspects or embodiments provided herein, the acquiring knowledge of one or more mutations in a CD274 gene comprises detecting the one or more mutations in a CD274 gene in the sample. In some embodiments of any of the aspects or embodiments provided herein, the methods further comprise selectively enriching for one or more nucleic acids comprising nucleotide sequences that comprise the one or more mutations in the CD274 gene; wherein the selectively enriching produces an enriched sample. In some embodiments of any of the aspects or embodiments provided herein, the one or more mutations in the CD274 gene are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments of any of the aspects or embodiments provided herein, the one or more mutations in the CD274 gene are detected in a PD-L1 polypeptide encoded by the CD274 gene. In some embodiments, the one or more mutations in the CD274 gene are detected in the sample by one or more of: immunoblotting, enzyme linked immunosorbent assay (ELISA), immunohistochemistry, or mass spectrometry.

In some embodiments of any of the aspects or embodiments provided herein, the cancer is a carcinoma, a sarcoma, a lymphoma, a leukemia, a myeloma, a germ cell cancer, or a blastoma. In some embodiments of any of the aspects or embodiments provided herein, the cancer is a solid tumor. In some embodiments of any of the aspects or embodiments provided herein, the cancer is a hematologic malignancy. In some embodiments of any of the aspects or embodiments provided herein, the cancer is a cancer listed in Table 5 or Table 6. In some embodiments of any of the aspects or embodiments provided herein, the cancer is diffuse large B-cell lymphoma, cutaneous squamous cell carcinoma, endometrial adenocarcinoma, unknown primary melanoma, or cutaneous melanoma.

In some embodiments of any of the aspects or embodiments provided herein, the cancer is a skin cancer. In some embodiments, the cancer comprises a tumor mutational burden (TMB) of ≥10 mutations/Megabase (mut/Mb). In some embodiments, the cancer comprises a TMB of less than 10 mut/Mb. In some embodiments, TMB is assessed based on about 0.79 megabases (Mb) of sequenced DNA. In some embodiments, TMB is assessed based on about 0.80 Mb of sequenced DNA. In some embodiments, TMB is assessed based on between about 0.83 Mb and about 1.14 Mb of sequenced DNA. In some embodiments, TMB is assessed based on about 1.1 Mb of sequenced DNA. In some embodiments, TMB is assessed based on up to about 1.24 Mb of sequenced DNA. In some embodiments, TMB is assessed based on up to about 1.1 Mb of sequenced DNA. In some embodiments, the cancer comprises a TMB of at least about 100 mut/Mb, at least about 110 mut/Mb, at least about 120 mut/Mb, at least about 130 mut/Mb, at least about 140 mut/Mb, at least about 150 mut/Mb, or more. In some embodiments, TMB is assessed by sequencing, whole exome sequencing, whole genome sequencing, gene-targeted sequencing, or next-generation sequencing. In some embodiments, the cancer is cutaneous squamous cell carcinoma, cutaneous melanoma, or unknown primary melanoma.

In some embodiments of any of the aspects or embodiments provided herein, the cancer is a non-serous endometrial adenocarcinoma. In some embodiments, the cancer comprises a high microsatellite instability status (MSI). In some embodiments, MSI is assessed based on DNA sequencing of up to about 114 loci.

In some embodiments of any of the aspects or embodiments provided herein, the cancer is a cancer comprising a CD274 mutation as listed in Table 6.

In some embodiments of any of the aspects or embodiments provided herein, the cancer is metastatic.

In some embodiments of any of the aspects or embodiments provided herein, the sample is obtained from the cancer. In some embodiments, the sample is a formalin-fixed paraffin-embedded (FFPE) sample. In some embodiments, the sample comprises fluid, cells, or tissue. In some embodiments, the sample comprises a tumor biopsy or a circulating tumor cell. In some embodiments, the sample is a nucleic acid sample. In some embodiments, the nucleic acid sample comprises mRNA, genomic DNA, circulating tumor DNA, cell-free DNA, or cell-free RNA. In some embodiments, the sample comprises one or more nucleic acids obtained from an FFPE sample from the individual. In some embodiments, the one or more nucleic acids comprise mRNA, genomic DNA, circulating tumor DNA, cell-free DNA, or cell-free RNA. In some embodiments, the methods provided herein further comprise obtaining more than one sample from the individual at different time points.

In some embodiments of any of the aspects or embodiments provided herein, the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample. In some embodiments, the bait comprises a capture nucleic acid molecule configured to hybridize to the one or more nucleic acids. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and 200 nucleotides. In some embodiments, the bait is conjugated to an affinity reagent or to a detection reagent. In some embodiments, the affinity reagent is an antibody, an antibody fragment, or biotin, or wherein the detection reagent is a fluorescent marker. In some embodiments, the capture nucleic acid molecule comprises a DNA, RNA, or mixed DNA/RNA molecule. In some embodiments, the methods provided herein further comprise sequencing the one or more nucleic acid molecules in the enriched sample.

In some embodiments of any of the aspects or embodiments provided herein, the selectively enriching comprises amplifying the one or more nucleic acids in the sample using a polymerase chain reaction (PCR) to produce the enriched sample. In some embodiments, the methods provided herein further comprise sequencing the one or more nucleic acid molecules in the enriched sample.

In some aspects, provided herein is a kit comprising a probe or bait for detecting one or more mutations in a CD274 gene, optionally wherein the one or more mutations in a CD274 gene comprise one or more mutations listed in Tables 1-4 and 6.

In some aspects, provided herein is a nucleic acid encoding a CD274 gene comprising one or more mutations listed in Tables 1-4 and 6.

In some aspects, provided herein is a vector comprising a nucleic acid provided herein.

In some aspects, provided herein is a host cell comprising a vector provided herein.

In some aspects, provided herein is an antibody or antibody fragment that specifically binds to a PD-L1 polypeptide encoded by a CD274 gene comprising one or more mutations listed in Tables 1-4 and 6.

In some aspects, provided herein is a kit comprising an antibody or antibody fragment provided herein.

In some aspects, provided herein is an in vitro use of one or more oligonucleotides for detecting a CD274 gene, or a portion thereof, comprising one or more mutations listed in Tables 1-4 and 6.

In some aspects, provided herein is a kit comprising one or more oligonucleotides for detecting a CD274 gene, or a portion thereof, comprising one or more mutations listed in Tables 1-4 and 6.

In some aspects, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of one or more mutations in a CD274 gene; and (c) detect, based on the analyzing, one or more mutations in a CD274 gene, in the sample. In some embodiments, the one or more mutations in a CD274 gene comprise one or more of a missense mutation, a truncation, a nonsense mutation, a splice site mutation, an insertion/deletion, and any combination thereof. In some embodiments, the one or more mutations in a CD274 gene comprise two or more missense mutations. In some embodiments, the one or more mutations in a CD274 gene comprise one or more missense mutations and a truncation. In some embodiments, the one or more mutations in a CD274 gene comprise one or more mutations listed in Tables 1-4 and 6. In some embodiments, the one or more mutations in a CD274 gene further comprise a CD274 gene amplification. In some embodiments, the one or more mutations in a CD274 gene further comprise a CD274 gene deletion, wherein the CD274 gene is a deletion of a portion of the CD274 gene. In some embodiments, the one or more mutations in a CD274 gene further comprise a CD274 genomic rearrangement or a CD274 gene fusion. In some embodiments, the one or more mutations in a CD274 gene are somatic mutations or germline mutations. In some embodiments, the one or more mutations in a CD274 gene are clonal mutations. In some embodiments, the one or more mutations in a CD274 gene are sub-clonal mutations. In some embodiments, the plurality of sequence reads is obtained by sequencing, whole exome sequencing, whole genome sequencing, gene-targeted sequencing, or next-generation sequencing.

In some aspects, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of one or more mutations in a CD274 gene; and (c) detecting, using the one or more processors and based on the analyzing, one or more mutations in a CD274 gene, in the sample. In some embodiments, the one or more mutations in a CD274 gene comprise one or more of a missense mutation, a truncation, a nonsense mutation, a splice site mutation, an insertion/deletion, and any combination thereof. In some embodiments, the one or more mutations in a CD274 gene comprise two or more missense mutations. In some embodiments, the one or more mutations in a CD274 gene comprise one or more missense mutations and a truncation. In some embodiments, the one or more mutations in a CD274 gene comprise one or more mutations listed in Tables 1-4 and 6. In some embodiments, the one or more mutations in a CD274 gene further comprise a CD274 gene amplification. In some embodiments, the one or more mutations in a CD274 gene further comprise a CD274 gene deletion, wherein the CD274 gene is a deletion of a portion of the CD274 gene. In some embodiments, the one or more mutations in a CD274 gene further comprise a CD274 genomic rearrangement or a CD274 gene fusion. In some embodiments, the one or more mutations in a CD274 gene are somatic mutations or germline mutations. In some embodiments, the one or more mutations in a CD274 gene are clonal mutations. In some embodiments, the one or more mutations in a CD274 gene are sub-clonal mutations. In some embodiments, the plurality of sequence reads is obtained by sequencing, whole exome sequencing, whole genome sequencing, gene-targeted sequencing, or next-generation sequencing.

In some aspects, provided herein is an anti-cancer therapy for use in a method of treating or delaying progression of cancer, wherein the method comprises administering the anti-cancer therapy to an individual, wherein one or more mutations in a CD274 gene are detected in a sample obtained from the individual.

In some aspects, provided herein is an anti-cancer therapy for use in the manufacture of a medicament for treating or delaying progression of cancer, wherein the medicament is to be administered to an individual, wherein one or more mutations in a CD274 gene are detected in a sample obtained from the individual.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

FIG. 1 provides an overview of the comprehensive genomic profiling (CGP) methods used to profile the landscape of CD274 mutations, as described in Example 1.

FIG. 2 is a lollipop plot of all the missense and nonsense mutations identified in the CGP analysis described in Example 1. The most common CD274 mutations were R260H (n=51), R260C (n=18), R125Q (n=12), C272fs*13 (n=11), R86W (n=10), and R113H (n=10).

FIG. 3 is a longtail plot showing the prevalence of CD274 mutations in different tumor types. Tumor types (at least 800 total samples) with the highest rates of CD274 mutations in descending order were: diffuse large b-cell lymphoma, cutaneous squamous cell carcinoma, uterine endometrial adenocarcinoma, unknown primary melanoma, and cutaneous melanoma.

FIGS. 4A-4C show the correlation of CD274 non-truncating mutations with PD-L1 immunohistochemistry (IHC) tumor cell expression. FIG. 4A shows that among non-truncating variants, 181 samples with missense substitutions and two in-frame indels were identified (lower panel). A subset of the variants was recurrent, with 12 samples harboring a substitution at R260. PD-L1 TPS scores corresponding to each sample with a non-truncating variant shown are provided in the upper panel. Sub-clonal variants are denoted with square markers. FIG. 4B shows the correlation of CD274 missense mutations and PD-L1 protein expression in mutations where at least 2 cases were analyzed for PD-L1 expression by IHC. FIG. 4C shows a comparison of PD-L1 expression in the clonal (n=153) versus sub-clonal (n=28) missense mutations. A significantly lower PD-L1 IHC expression in the clonal missense mutations compared to sub-clonal missense mutations was observed (mean: TPS=11 vs 22, respectively; ANOVA, p=0.049). Sub-clonal variants were defined as samples where <50% of tumor cells were predicted to harbor the variant based on the variant allele fraction and the pathologic and/or computational tumor cell purity estimates.

FIGS. 5A-5B show the correlation of CD274 truncating mutations with PD-L1 immunohistochemistry (IHC) tumor cell expression. FIG. 5A shows 39 identified putative truncating variants, including 12 nonsense mutations, 10 frame shift indels, and seven canonical splice variants (lower panel). PD-L1 TPS scores corresponding to each sample with a truncating variant shown are provided in the upper panel. Sub-clonal variants are denoted with square markers. FIG. 5B shows a comparison of PD-L1 expression in samples with clonal truncating variants (nonsense or frame shift indel) and sub-clonal truncating variants. A significantly lower level of PD-L1 expression in samples with clonal truncating variants (nonsense or frame shift indel) was observed when compared to samples with sub-clonal truncating variants (mean: TPS=1 vs TPS=38; ANOVA, p<0.001). Sub-clonal variants were defined as samples where <50% of tumor cells were predicted to harbor the variant based on the variant allele fraction and the pathologic and/or computational tumor cell purity estimates.

FIG. 6 shows the correlation between the predicted functionality of missense CD274 mutations (assessed by Meta SVM Rankscore) and PD-L1 TPS score.

FIG. 7 depicts an exemplary device, “Device 1100,” in accordance with some embodiments.

FIG. 8 depicts an exemplary system, “System 1200,” in accordance with some embodiments.

FIG. 9 depicts a block diagram of an exemplary process for detecting one or more mutations in a CD274 gene, in accordance with some embodiments.

### DETAILED DESCRIPTION

The present disclosure relates generally to detecting one or more mutations in a CD274 gene, as well as methods of treatment, uses, and kits related thereto.

The present disclosure describes comprehensive genomic profiling undertaken on a large pan-cancer cohort of 314,631 samples, which revealed 1,081 cases with CD274 mutations. Identified mutations included, without limitation, missense mutations, nonsense mutations and insertion/deletion alterations. Without wishing to be bound by theory, it is thought that CD274 mutations can mediate resistance to immune checkpoint inhibitors (ICPI) due to, for example and without limitation, steric or affinity-altering interferences in the binding of the PD-L1 ligand to the PD-1 receptor.

The present disclosure further describes an analysis of PD-L1 protein expression in a subset of cancer cases with CD274 mutations (N=213), revealing that the expression levels of PD-L1 differed among samples comprising the various identified CD274 mutations. Certain CD274 mutations were associated with no PD-L1 protein expression, low PD-L1 protein expression, or high PD-L1 protein expression. Without wishing to be bound by theory, it is thought that CD274 mutations associated with low or no PD-L1 expression can act as resistance biomarkers for ICPI due to, for example and without limitation, the lack of PD-L1 protein present on the tumor cells, and/or loss of affinity for binding of PD-L1 antibodies used to detect PD-L1 protein expression and therefore loss of affinity for binding of anti-PD-L1 therapies.

### I. GENERAL TECHNIQUES

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N. Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

### II. DEFINITIONS

As used in this specification and the appended claims, the singular forms “a”, “an” and “the” include plural referents unless the context clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The terms “cancer” and “cancerous” refer to or describe the physiological condition in mammals that is typically characterized by unregulated cell growth. Included in this definition are benign and malignant cancers.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

As used herein, the terms “cluster of differentiation 274” or “CD274” refer to a gene encoding a programmed death-ligand 1 (PD-L1) mRNA or a PD-L1 polypeptide. CD274 is a gene located on chromosome 9p24.1. CD274 is also known as B7-H, B7-H1, B7H1, PD-L1, PDCD1LG1, PDL1. In some embodiments, a CD274 gene is a human CD274 gene.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2-0-methyl-, 2-0-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “isolated” antibody is one which has been identified and separated and/or recovered from a component of its natural environment. Contaminant components of its natural environment are materials which would interfere with research, diagnostic, and/or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. An isolated antibody includes the antibody in situ within recombinant cells since at least one component of the antibody's natural environment will not be present. Ordinarily, however, an isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “light chains” of antibodies (immunoglobulins) from any mammalian species can be assigned to one of two clearly distinct types, called kappa (“w”) and lambda (“k”), based on the amino acid sequences of their constant domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2, and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The term “hypervariable region,” “HVR,” or “HV,” as used herein, refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, for example, Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, for example, Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | numbering) |
| H1 | H31-H35 | H26-H35 | H26-H32 | H30-H35 (Chothia |
| | | | | numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody comprising the antigen-binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target-binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target-binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target-binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101 (34): 12467-12472 (2004); and Lee et al., J. Immunol. Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg et al., Intern. Rev. Immunol. 13: 65-93 (1995)).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody.

A “humanized form” of an antibody, e.g., a non-human antibody, refers to an antibody that has undergone humanization.

A “blocking” antibody or an “antagonist” antibody is one which inhibits or reduces biological activity of the antigen it binds. For example, blocking antibodies or antagonist antibodies substantially or completely inhibit the biological activity of the antigen.

As used herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 1 0% of the binding of the antibody to the target as measured, e.g., by a radioimmunoassay (RIA). In certain embodiments, an antibody that specifically binds to a target has a dissociation constant (Kd) of <1 μM, <100 nM, <10 nM, <1 nM, or <0.1 nM. In certain embodiments, an antibody specifically binds to an epitope on a protein that is conserved among the protein from different species. In another embodiment, specific binding can include, but does not require exclusive binding.

“Percent (%) amino acid sequence identity” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, California. The ALIGN-2 program should be compiled for use on a UNIX operating system, for example, digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “detection” includes any means of detecting, including direct and indirect detection. The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy including a checkpoint inhibitor). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations), polypeptides, polypeptide and polynucleotide modifications (e.g., post-translational modifications), carbohydrates, and/or glycolipid-based molecular markers.

The “amount” or “number” of somatic mutations associated with an increased clinical benefit to an individual is a detectable level in a biological sample. These can be measured by methods known to one skilled in the art and also disclosed herein. The amount of a somatic mutation assessed can be used to determine the response to the treatment.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, N Y, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain somatic mutations in a biological sample from an individual.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some instances, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refers to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and in some embodiments stop) cancer cell infiltration into peripheral organs; inhibit (i.e., slow to some extent and in some embodiments stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

As used herein, “administering” is meant a method of giving a dosage of a compound (e.g., an antagonist) or a pharmaceutical composition (e.g., a pharmaceutical composition including an antagonist) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker (e.g., a one or more mutations in a CD274 gene) described herein. In certain embodiments, the manufacture or kit is promoted, distributed, or sold as a unit for performing the methods described herein.

The phrase “based on” when used herein means that the information about one or more biomarkers (e.g., a one or more mutations in a CD274 gene) is used to inform a treatment decision, information provided on a package insert, or marketing/promotional guidance, etc.

### III. METHODS, SYSTEMS, AND DEVICES

In one aspect, provided herein are methods of identifying an individual having cancer who may or may not benefit from a treatment comprising an anti-cancer therapy. In some embodiments, the methods comprise detecting one or more mutations in a CD274 gene in a sample from the individual, wherein the presence of the one or more mutations in a CD274 gene in the sample identifies the individual as one who may benefit from an anti-cancer therapy. In some embodiments, the presence of the one or more mutations in the CD274 gene in the sample identifies the individual as one who may have a cancer that is resistant to one or more immune checkpoint inhibitors.

In another aspect, provided herein are methods of detecting the presence or absence of a cancer in an individual. In some embodiments, the methods comprise: (a) detecting the presence or absence of a cancer in a sample from the individual; and (b) detecting the presence or absence of one or more mutations in a CD274 gene in the sample.

In another aspect, provided herein are methods of selecting a therapy for an individual having cancer. In some embodiments, the methods comprise detecting one or more mutations in a CD274 gene in a sample from the individual, wherein the presence of the one or more mutations in a CD274 gene in the sample identifies the individual as one who may benefit from an anti-cancer therapy. In some embodiments, the presence of the one or more mutations in the CD274 gene in the sample identifies the individual as one who may have a cancer that is resistant to one or more immune checkpoint inhibitors.

In another aspect, provided herein are methods of identifying one or more treatment options for an individual having cancer. In some embodiments, the methods comprise detecting, or acquiring knowledge of, one or more mutations in a CD274 gene in a sample from the individual and generating a report comprising one or more treatment options identified for the individual based at least in part on the presence of the one or more mutations in a CD274 gene in the sample, wherein the one or more treatment options comprise an anti-cancer therapy. In some embodiments, the report identifies the individual as one who may have a cancer that is resistant to one or more immune checkpoint inhibitors.

In another aspect, provided herein are methods of selecting or not selecting treatment for an individual having cancer. In some embodiments, the methods comprise acquiring knowledge of one or more mutations in a CD274 gene in a sample from an individual having cancer, wherein responsive to the acquisition of said knowledge: (i) the individual is classified as a candidate to receive treatment with an anti-cancer therapy, or the individual is not classified as a candidate to receive treatment with an anti-cancer therapy; and/or (ii) the individual is identified as likely to respond to a treatment that comprises an anti-cancer therapy, or the individual is identified as unlikely to respond to a treatment that comprises an anti-cancer therapy. In some embodiments, responsive to the acquisition of said knowledge: (i) the individual is classified as having a cancer that is resistant to one or more immune checkpoint inhibitors; and/or (ii) the individual is identified as unlikely to respond to a treatment that comprises one or more immune checkpoint inhibitors.

In another aspect, provided herein are methods of treating or delaying progression of cancer. In some embodiments, the methods comprise administering to an individual an effective amount of an anti-cancer therapy, wherein the cancer comprises one or more mutations in a CD274 gene. In some embodiments, the methods comprise, responsive to knowledge of one or more mutations in a CD274 gene in a sample from an individual, administering to the individual an effective amount of an anti-cancer therapy. In some embodiments, the methods comprise detecting or acquiring knowledge of one or more mutations in a CD274 gene in a sample from the individual. In some embodiments, the methods comprise detecting one or more mutations in a CD274 gene in a sample from the individual and administering to the individual an effective amount of an anti-cancer therapy.

In another aspect, provided herein are methods of monitoring an individual having cancer. In some embodiments, the methods comprise acquiring knowledge of one or more mutations in a CD274 gene in a sample from the individual, wherein responsive to the acquisition of said knowledge, the individual is predicted to have increased risk of cancer resistance to one or more immune checkpoint inhibitors, e.g., as compared to an individual whose cancer does not comprise one or more mutations in a CD274 gene.

In another aspect, provided herein are methods of predicting survival of an individual having cancer. In some embodiments, the methods comprise acquiring knowledge of one or more mutations in a CD274 gene in a sample from the individual, wherein responsive to the acquisition of said knowledge, the individual is predicted to have shorter survival when treated with one or more immune checkpoint inhibitors, e.g., as compared to survival of an individual whose cancer does not comprise one or more mutations in a CD274 gene.

In another aspect, provided herein are methods of evaluating an individual having cancer. In some embodiments, the methods comprise acquiring knowledge of one or more mutations in a CD274 gene in a sample from the individual, wherein responsive to the acquisition of said knowledge, the individual is predicted to have increased risk of cancer that is resistant to one or more immune checkpoint inhibitors, e.g., as compared to an individual whose cancer does not comprise one or more mutations in a CD274 gene.

In another aspect, provided herein are methods of screening an individual having cancer. In some embodiments, the methods comprise acquiring knowledge of one or more mutations in a CD274 gene in a sample from the individual, wherein responsive to the acquisition of said knowledge, the individual is predicted to have increased risk of cancer that is resistant to one or more immune checkpoint inhibitors, e.g., as compared to an individual whose cancer does not comprise one or more mutations in a CD274 gene.

In another aspect, provided herein is a method of predicting survival of an individual having a cancer treated with one or more immune checkpoint inhibitors. In some embodiments, the method comprises acquiring knowledge of one or more mutations in a CD274 gene in a sample from the individual, wherein responsive to the acquisition of said knowledge, the individual is predicted to have shorter survival after treatment with the one or more immune checkpoint inhibitors, as compared to an individual whose cancer does not exhibit one or more mutations in a CD274 gene.

In another aspect, provided herein are methods of detecting one or more mutations in a CD274 gene. In some embodiments, the methods comprise detecting one or more mutations in a CD274 gene in a sample from an individual.

In another aspect, provided herein are methods of diagnosing or assessing one or more mutations in a CD274 gene. In some embodiments, the methods comprise detecting one or more mutations in a CD274 gene in a sample from an individual and providing a diagnosis/assessment of one or more mutations in a CD274 gene.

In another aspect, provided herein are methods of diagnosing an immune checkpoint inhibitor-resistant cancer in an individual. In some embodiments, the methods comprise detecting one or more mutations in a CD274 gene in a sample from an individual and optionally providing a diagnosis of an immune checkpoint inhibitor-resistant cancer in the individual.

In another aspect, provided herein are methods of detecting one or more mutations in a CD274 gene, e.g., in a sample from an individual. In some embodiments, the methods comprise providing a plurality of nucleic acids obtained from a sample from an individual, wherein the plurality of nucleic acids comprises nucleic acids encoding a CD274 gene, or a portion thereof; optionally, ligating one or more adaptors onto one or more nucleic acids from the plurality of nucleic acids; optionally, amplifying nucleic acids from the plurality of nucleic acids; optionally, capturing a plurality of nucleic acids corresponding to the CD274 gene; sequencing, by a sequencer, the plurality of nucleic acids to obtain a plurality of sequence reads corresponding to the CD274 gene; analyzing the plurality of sequence reads; and based on the analysis, detecting one or more mutations in the CD274 gene. In some embodiments, the plurality of nucleic acids corresponding to the CD274 gene are captured from the amplified nucleic acids by hybridization with a bait molecule. In some embodiments, the methods comprise providing a sample from an individual having a cancer, wherein the sample comprises one or more nucleic acids; preparing a nucleic acid sequencing library from the one or more nucleic acids in the sample; amplifying said library using a polymerase chain reaction (PCR); selectively enriching for one or more nucleic acids comprising CD274 nucleotide sequences in said library to produce an enriched sample; sequencing the enriched sample, thereby producing a plurality of sequencing reads; analyzing the plurality of sequencing reads for the presence of one or more mutations in a CD274 gene; and detecting, based on the analyzing step, one or more mutations in a CD274 gene in the sample from the individual.

In another aspect, provided herein are uses (e.g., in vitro uses) of one or more oligonucleotides for detecting one or more mutations in a CD274 gene.

In another aspect, provided herein are kits or articles of manufacture comprising one or more oligonucleotides for detecting one or more mutations in a CD274 gene.

In another aspect, provided herein are kits or articles of manufacture comprising an anti-cancer therapy and a package insert comprising instructions for using the anti-cancer therapy in a method of treating or delaying progression of cancer, e.g., by administration to an individual from whom a sample comprising one or more mutations in a CD274 gene has been obtained.

In another aspect, provided herein are anti-cancer therapies for use in a method of treating or delaying progression of cancer. In some embodiments, the method comprises administering the anti-cancer therapies to an individual, wherein one or more mutations in a CD274 gene have been detected in a sample from the individual.

In another aspect, provided herein are anti-cancer therapies for use in the manufacture of a medicament for treating or delaying progression of cancer, e.g., in an individual from whom a sample comprising one or more mutations in a CD274 gene has been obtained. In some embodiments, the method comprises administering the anti-cancer therapy to an individual, wherein one or more mutations in a CD274 gene have been detected in a sample from the individual.

In another aspect, provided herein are systems, e.g., comprising a memory and one or more processors. In some embodiments, the systems comprise a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of one or more mutations in a CD274 gene; and (c) detect, based on the analyzing, one or more mutations in a CD274 gene, in the sample.

In another aspect, provided herein are computer-readable storage media. In some embodiments, the computer-readable storage media comprise one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of one or more mutations in a CD274 gene; and (c) detecting, using the one or more processors and based on the analyzing, one or more mutations in a CD274 gene in the sample. In some embodiments, the computer-readable storage media are non-transitory. In some embodiments, the computer-readable storage media are transitory.

### CD274 Mutations

The CD274 gene, located on chromosome 9p24.1, encodes programmed death-ligand 1 (PD-L1) protein. The CD274 Matched Annotation from the U.S. National Center for Biotechnology Information (NCBI) and the European Bioinformatics Institute (EMBL-EBI) (Matched Annotation from NCBI and EMBL-EBI: MANE) transcript (ENST00000381577.4) encodes for a type 1 transmembrane protein that is 290 amino acids long and has immunoglobulin V-like and C-like domains.

An exemplary CD274 gene is represented by NCBI Gene ID No. 29126. An exemplary CD274 mRNA sequence is represented by NCBI Ref. Seq. NM_014143:

(SEQ ID NO: 1)
AGTTCTGCGCAGCTTCCCGAGGCTCCGCACCAGCCGCGCTTCTGTCCGCCTGCAGGGCAT
TCCAGAAAGATGAGGATATTTGCTGTCTTTATATTCATGACCTACTGGCATTTGCTGAAC
GCATTTACTGTCACGGTTCCCAAGGACCTATATGTGGTAGAGTATGGTAGCAATATGACA
ATTGAATGCAAATTCCCAGTAGAAAAACAATTAGACCTGGCTGCACTAATTGTCTATTGG
GAAATGGAGGATAAGAACATTATTCAATTTGTGCATGGAGAGGAAGACCTGAAGGTTCA
GCATAGTAGCTACAGACAGAGGGCCCGGCTGTTGAAGGACCAGCTCTCCCTGGGAAATG
CTGCACTTCAGATCACAGATGTGAAATTGCAGGATGCAGGGGTGTACCGCTGCATGATC
AGCTATGGTGGTGCCGACTACAAGCGAATTACTGTGAAAGTCAATGCCCCATACAACAA
AATCAACCAAAGAATTTTGGTTGTGGATCCAGTCACCTCTGAACATGAACTGACATGTCA
GGCTGAGGGCTACCCCAAGGCCGAAGTCATCTGGACAAGCAGTGACCATCAAGTCCTGA
GTGGTAAGACCACCACCACCAATTCCAAGAGAGAGGAGAAGCTTTTCAATGTGACCAGC
ACACTGAGAATCAACACAACAACTAATGAGATTTTCTACTGCACTTTTAGGAGATTAGAT
CCTGAGGAAAACCATACAGCTGAATTGGTCATCCCAGAACTACCTCTGGCACATCCTCCA
AATGAAAGGACTCACTTGGTAATTCTGGGAGCCATCTTATTATGCCTTGGTGTAGCACTG
ACATTCATCTTCCGTTTAAGAAAAGGGAGAATGATGGATGTGAAAAAATGTGGCATCCA
AGATACAAACTCAAAGAAGCAAAGTGATACACATTTGGAGGAGACGTAATCCAGCATTG
GAACTTCTGATCTTCAAGCAGGGATTCTCAACCTGTGGTTTAGGGGTTCATCGGGGCTGA
GCGTGACAAGAGGAAGGAATGGGCCCGTGGGATGCAGGCAATGTGGGACTTAAAAGGC
CCAAGCACTGAAAATGGAACCTGGCGAAAGCAGAGGAGGAGAATGAAGAAAGATGGAG
TCAAACAGGGAGCCTGGAGGGAGACCTTGATACTTTCAAATGCCTGAGGGGCTCATCGA
CGCCTGTGACAGGGAGAAAGGATACTTCTGAACAAGGAGCCTCCAAGCAAATCATCCAT
TGCTCATCCTAGGAAGACGGGTTGAGAATCCCTAATTTGAGGGTCAGTTCCTGCAGAAGT
GCCCTTTGCCTCCACTCAATGCCTCAATTTGTTTTCTGCATGACTGAGAGTCTCAGTGTTG
GAACGGGACAGTATTTATGTATGAGTTTTTCCTATTTATTTTGAGTCTGTGAGGTCTTCTT
GTCATGTGAGTGTGGTTGTGAATGATTTCTTTTGAAGATATATTGTAGTAGATGTTACAAT
TTTGTCGCCAAACTAAACTTGCTGCTTAATGATTTGCTCACATCTAGTAAAACATGGAGT
ATTTGTAAGGTGCTTGGTCTCCTCTATAACTACAAGTATACATTGGAAGCATAAAGATCA
AACCGTTGGTTGCATAGGATGTCACCTTTATTTAACCCATTAATACTCTGGTTGACCTAAT
CTTATTCTCAGACCTCAAGTGTCTGTGCAGTATCTGTTCCATTTAAATATCAGCTTTACAA
TTATGTGGTAGCCTACACACATAATCTCATTTCATCGCTGTAACCACCCTGTTGTGATAAC
CACTATTATTTTACCCATCGTACAGCTGAGGAAGCAAACAGATTAAGTAACTTGCCCAAA
CCAGTAAATAGCAGACCTCAGACTGCCACCCACTGTCCTTTTATAATACAATTTACAGCT
ATATTTTACTTTAAGCAATTCTTTTATTCAAAAACCATTTATTAAGTGCCCTTGCAATATC
AATCGCTGTGCCAGGCATTGAATCTACAGATGTGAGCAAGACAAAGTACCTGTCCTCAA
GGAGCTCATAGTATAATGAGGAGATTAACAAGAAAATGTATTATTACAATTTAGTCCAGT
GTCATAGCATAAGGATGATGCGAGGGGAAAACCCGAGCAGTGTTGCCAAGAGGAGGAA
ATAGGCCAATGTGGTCTGGGACGGTTGGATATACTTAAACATCTTAATAATCAGAGTAAT
TTTCATTTACAAAGAGAGGTCGGTACTTAAAATAACCCTGAAAAATAACACTGGAATTCC
TTTTCTAGCATTATATTTATTCCTGATTTGCCTTTGCCATATAATCTAATGCTTGTTTATAT
AGTGTCTGGTATTGTTTAACAGTTCTGTCTTTTCTATTTAAATGCCACTAAATTTTAAATT
CATACCTTTCCATGATTCAAAATTCAAAAGATCCCATGGGAGATGGTTGGAAAATCTCCA
CTTCATCCTCCAAGCCATTCAAGTTTCCTTTCCAGAAGCAACTGCTACTGCCTTTCATTCA
TATGTTCTTCTAAAGATAGTCTACATTTGGAAATGTATGTTAAAAGCACGTATTTTTAAA
ATTTTTTTCCTAAATAGTAACACATTGTATGTCTGCTGTGTACTTTGCTATTTTTATTTATT
TTAGTGTTTCTTATATAGCAGATGGAATGAATTTGAAGTTCCCAGGGCTGAGGATCCATG
CCTTCTTTGTTTCTAAGTTATCTTTCCCATAGCTTTTCATTATCTTTCATATGATCCAGTAT
ATGTTAAATATGTCCTACATATACATTTAGACAACCACCATTTGTTAAGTATTTGCTCTAG
GACAGAGTTTGGATTTGTTTATGTTTGCTCAAAAGGAGACCCATGGGCTCTCCAGGGTGC
ACTGAGTCAATCTAGTCCTAAAAAGCAATCTTATTATTAACTCTGTATGACAGAATCATG
TCTGGAACTTTTGTTTTCTGCTTTCTGTCAAGTATAAACTTCACTTTGATGCTGTACTTGCA
AAATCACATTTTCTTTCTGGAAATTCCGGCAGTGTACCTTGACTGCTAGCTACCCTGTGCC
AGAAAAGCCTCATTCGTTGTGCTTGAACCCTTGAATGCCACCAGCTGTCATCACTACACA
GCCCTCCTAAGAGGCTTCCTGGAGGTTTCGAGATTCAGATGCCCTGGGAGATCCCAGAGT
TTCCTTTCCCTCTTGGCCATATTCTGGTGTCAATGACAAGGAGTACCTTGGCTTTGCCACA
TGTCAAGGCTGAAGAAACAGTGTCTCCAACAGAGCTCCTTGTGTTATCTGTTTGTACATG
TGCATTTGTACAGTAATTGGTGTGACAGTGTTCTTTGTGTGAATTACAGGCAAGAATTGT
GGCTGAGCAAGGCACATAGTCTACTCAGTCTATTCCTAAGTCCTAACTCCTCCTTGTGGT
GTTGGATTTGTAAGGCACTTTATCCCTTTTGTCTCATGTTTCATCGTAAATGGCATAGGCA
GAGATGATACCTAATTCTGCATTTGATTGTCACTTTTTGTACCTGCATTAATTTAATAAAA
TATTCTTATTTATTTTGTTACTTGGTACACCAGCATGTCCATTTTCTTGTTTATTTTGTGTTT
AATAAAATGTTCAGTTTAACATCCCA

An exemplary PD-L1 polypeptide is represented by NCBI Protein ID No. NP_054862.1. An exemplary PD-L1 amino acid sequence is represented by NCBI Ref. Seq. NP_054862.1:

(SEQ ID NO: 2)
MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLFNVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET

Certain aspects of the present disclosure relate to detection of one or more mutations in a CD274 gene.

In some embodiments, the one or more mutations in a CD274 gene comprise one or more of a substitution of one or more nucleotides, an insertion of one or more nucleotides, or a deletion of one or more nucleotides. In some embodiments, the one or more mutations comprise one or more of a missense mutation, a truncation, a nonsense mutation, a splice site mutation, an insertion/deletion (e.g., an indel), and any combination thereof. In some embodiments, the one or more mutations comprise two or more missense mutations (e.g., any of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more missense mutations). In some embodiments, the one or more mutations comprise one or more missense mutations and a truncation. In some embodiments, a truncation mutation is a nonsense mutation, a substitution of a codon for a stop codon, a frameshift mutation, or an indel. In some embodiments, the one or more mutations comprise one or more of a genomic rearrangement, an alteration in a promoter, a gene fusion, or a copy number alteration. In some embodiments, the one or more mutations comprise a gene copy number alteration. In some embodiments, the one or more mutations comprise a gene amplification. In some embodiments, the one or more mutations comprise a gene deletion, e.g., a deletion of the entire gene or of a portion of the gene. In some embodiments, the one or more mutations comprise a point mutation. In some embodiments, the one or more mutations comprise a single nucleotide polymorphism. In some embodiments, the one or more mutations comprise one or more mutations in an exon and/or an intron of the gene. In some embodiments, the one or more mutations comprise a non-synonymous mutation. In some embodiments, the one or more mutations comprise a gain-of-function mutation, e.g., an activating mutation. In some embodiments, the one or more mutations comprise a loss-of-function mutation, e.g., an inactivating mutation. In some embodiments, the one or more mutations result in a frameshift. In some embodiments, the one or more mutations result in a premature stop codon. In some embodiments, the one or more mutations comprise a functional alteration. In some embodiments, the one or more mutations comprise a mutation that alters the function of the polypeptide or protein encoded by the gene. In some embodiments, the one or more mutations comprise a complex insertion. In some embodiments, the one or more mutations comprise a complex deletion. In some embodiments, the one or more mutations comprise a mutation in a splice site. In some embodiments, the one or more mutations alter the splicing of an mRNA molecule encoded by the gene. In some embodiments, the one or more mutations comprise an insertion of one or more nucleotides. In some embodiments, the insertion comprises an insertion of between about 1 and about 5 nucleotides, between about 5 and about 10 nucleotides, between about 10 and about 20 nucleotides, between about 20 and about 30 nucleotides, between about 30 and about 40 nucleotides, or between about 40 and about 50 nucleotides. In some embodiments, the insertion comprises an insertion of between about 50 and about 100 nucleotides, between about 100 and about 200 nucleotides, between about 200 and about 300 nucleotides, between about 300 and 400 nucleotides, between about 400 and about 500 nucleotides, between about 500 and about 600 nucleotides, between about 600 and about 700 nucleotides, between about 700 and about 800 nucleotides, between about 800 and about 900 nucleotides, or between about 900 and about 1000 nucleotides. In some embodiments, the insertion comprises an insertion of between about 1000 and about 1500 nucleotides, between about 1500 and about 2000 nucleotides, between about 2000 and about 2500 nucleotides, between about 2500 and about 3000 nucleotides, between about 3000 and about 3500 nucleotides, between about 3500 and about 4000 nucleotides, between about 4000 and about 4500 nucleotides, between about 4500 and about 5000 nucleotides, between about 5000 and about 5500 nucleotides, between about 5500 and about 6000 nucleotides, between about 6000 and about 6500 nucleotides, between about 6500 and about 7000 nucleotides, between about 7000 and about 7500 nucleotides, between about 7500 and about 8000 nucleotides, between about 8000 and about 8500 nucleotides, between about 8500 and about 9000 nucleotides, between about 9000 and about 9500 nucleotides, or between about 9500 and about 10000 nucleotides. In some embodiments, the one or more mutations comprise a deletion of one or more nucleotides. In some embodiments, the deletion comprises a deletion of between about 1 and about 5 nucleotides, between about 5 and about 10 nucleotides, between about 10 and about 20 nucleotides, between about 20 and about 30 nucleotides, between about 30 and about 40 nucleotides, or between about 40 and about 50 nucleotides. In some embodiments, the deletion comprises a deletion of between about 50 and about 100 nucleotides, between about 100 and about 200 nucleotides, between about 200 and about 300 nucleotides, between about 300 and 400 nucleotides, between about 400 and about 500 nucleotides, between about 500 and about 600 nucleotides, between about 600 and about 700 nucleotides, between about 700 and about 800 nucleotides, between about 800 and about 900 nucleotides, or between about 900 and about 1000 nucleotides. In some embodiments, the deletion comprises a deletion of between about 1000 and about 1500 nucleotides, between about 1500 and about 2000 nucleotides, between about 2000 and about 2500 nucleotides, between about 2500 and about 3000 nucleotides, between about 3000 and about 3500 nucleotides, between about 3500 and about 4000 nucleotides, between about 4000 and about 4500 nucleotides, between about 4500 and about 5000 nucleotides, between about 5000 and about 5500 nucleotides, between about 5500 and about 6000 nucleotides, between about 6000 and about 6500 nucleotides, between about 6500 and about 7000 nucleotides, between about 7000 and about 7500 nucleotides, between about 7500 and about 8000 nucleotides, between about 8000 and about 8500 nucleotides, between about 8500 and about 9000 nucleotides, between about 9000 and about 9500 nucleotides, or between about 9500 and about 10000 nucleotides. In some embodiments, the one or more mutations result in a substitution, insertion, or deletion of one or more amino acid residues in a polypeptide or a protein encoded by the gene. In some embodiments, the one or more mutations result in a substitution of one or more amino acid residues in a polypeptide or a protein encoded by the gene. In some embodiments, the one or more mutations result in a deletion of one or more amino acid residues in a polypeptide or a protein encoded by the gene. In some embodiments, the one or more mutations result in an insertion of one or more amino acid residues in a polypeptide or a protein encoded by the gene.

In some embodiments, the one or more mutations in a CD274 gene reduce the interaction between a PD-L1 polypeptide encoded by the CD274 gene and one or more PD-L1 ligands, e.g., PD-1 receptor or B7-1. In some embodiments, the one or more mutations in a CD274 gene reduce the interaction between a PD-L1 polypeptide encoded by the CD274 gene and a PD-1 receptor.

In some embodiments, the one or more mutations in a CD274 gene reduce the activity of a PD-L1 polypeptide encoded by the CD274 gene. In some embodiments, the one or more mutations in a CD274 gene result in an immune checkpoint inhibitor resistant cancer.

Exemplary and non-limiting CD274 gene mutations include those listed in Table 1.

TABLE 1
CD274 variants.

| Mutation Type | CD274 Mutation |
|---|---|
| Missense Mutations | R260H, R260C, R125Q, R86W, R113H, D215H, R140I, R140T, H233Y, A18T, |
| | R86Q, E223K, P24S, M266I, Y112C, S169N, A163V, G245E, E217Q, A232G, |
| | D284A, A85V, G177S, K280N, T290A, A52V, E158K, H220Y, K105Q, P235S, |
| | Q83H, S184F, R262I, E187Q, E217K, Q139R, K25N, M36I, Q173E, E205Q, |
| | D61N, F207L, G177D, K129N, D276Y, G119D, N183S, P146L, P43S, R140K, |
| | A232T, A232V, E187D, E289D, G95E, M1I, P230S, R265T, A109V, A5V, F9L, |
| | Q156H, R262K, R265K, W13C, A232S, P216S, S79N, T148A, D268N, G264E, |
| | M10I, P227S, R212K, E150K, G110R, K185N, L231V, P146S, S169C, D145Y, |
| | E237K, E58K, L16P, L229P, N135S, A132V, G95R, I258M, I274M, R113C, |
| | S279L, V253I, E218K, E188K, E223Q, E288Q, F67C, K75T, L241F, Q173H, |
| | Q47E, T179I, T201I, V193M, V21L, W57G, Y208C, D122N, D268H, D61Y, |
| | E71Q, G119S, G70A, H78R, K178E, L27I, N131I, P235L, Q107K, S169R, |
| | T210S, V21A, V242I, A157T, D284N, E164Q, L244M, L249V, Q66E, R2M, |
| | V165A, D268G, D26G, E288G, I8K, K62N, L142F, P133L, P133S, P216L, |
| | P43Q, T285I, V174G, V174I, V174L, V6I, A85T, C209Y, D103Y, D276N, |
| | E39K, E39Q, F257L, I8V, K75N, L106F, M59I, R198K, V143A, V147A, V30A, |
| | D108N, D122Y, D73N, D90Y, G159C, G177C, H14Y, I38T, L190F, L53P, |
| | R238K, T221A, V29M, V68E, V76F, C272Y, D171G, E188V, E71K, H69N, |
| | K25R, L92H, T196A, T221I, V242A, A51V, A97V, A98T, E45K, E45Q, F257C, |
| | H69R, P161L, P216H, Q139E, Q91H, S80R, T181A, T256S, V23F, V68A, |
| | Y160H, D90A, E152Q, E158V, E288K, E31G, F4S, G245A, G245V, G252D, |
| | G264V, G70R, I126F, I126L, I199V, I38M, I3T, K162N, K189Q, L197P, |
| | L244V, L50V, L94Q, N135D, N183D, N192I, P161S, P24A, Q275R, R113S, |
| | R238T, R265G, R82K, S283T, V111L, V253G, V269M, P234S, Q47R, R186G, |
| | R198T, R212I, S176G, T102I, T20A, T37K, V174A, V174D, Y28S, A121V, |
| | A222V, A246V, D215Y, D49G, D61V, E288D, F211C, G252C, G264R, H240L, |
| | I166V, I65M, K25M, K89N, L16R, L190I, L255R, L53I, M59T, N236D, P227A, |
| | P234R, Q100R, Q156E, Q83R, R2K, S195I, T154I, T182P, T22S, V128L, |
| | V130L, V174F, Y12F, Y32C, Y56C, C209F, C209S, D171N, D276E, I206M, |
| | I206T, I226L, I226M, I243V, K136E, M115L, N200S, N204I, N219T, N236I, |
| | N96S, P227T, Q100H, Q282R, Q91E, R198G, R262G, R2W, T203S, V143I, |
| | V44A, V55F, V68M, Y123H, A51D, A85S, A98V, C250G, C272R, D284Y, |
| | D49N, E152K, E158G, E188D, E218Q, G119V, I64V, K124N, K46R, L248S, |
| | L287V, M10T, M10V, M36T, M36V, N131K, N138T, N17D, N200D, N219I, |
| | N236H, S79G, T194A, T285S, W167C, W57S, Y118F, Y134C, A18S, A222G, |
| | A232P, A97T, C114Y, C250Y, D215N, E150G, E31A, F211L, G252S, G264W, |
| | H172Y, H240P, I258F, I64M, I64T, K281N, L190R, L249S, L251F, N131H, |
| | N138H, N138K, N35D, P43A, S195R, T127A, T180I, T202I, T202R, T239S, |
| | T37A, V23I, V29G, D145E, D61H, D73H, E164A, E187V, E237Q, F7L, |
| | G159R, H240Y, I141V, N35K, N96H, N96Y, P230L, P230T, R125L, S184P, |
| | S195N, T181I, T203A, T203I, T285A, V143F, V76I, A246D, D122E, D145H, |
| | E187K, E205K, E223V, E228V, E31K, F211I, F259L, G177V, G273D, H151L, |
| | H69L, I126S, I166L, I243T, I54L, K189E, K271N, K280Q, L190V, L231M, |
| | L244P, L255M, L50M, L53R, L88F, M115T, M267I, N204K, N35H, N63Y, |
| | P234F, P43L, R186K, R213K, R2G, R82I, A157S, A51S, D103N, D26N, D90E, |
| | E158Q, E58G, F67I, H151R, H172Q, H220D, I3M, K162R, K185E, K263E, |
| | L142W, L261F, N183H, P234T, Q156K, R113L, R186T, R212T, R265I, S149Y, |
| | T154S, T285P, V165L, Y81H, A132D, A254G, A5D, D103H, D145N, D26Y, |
| | D276H, D61E, E188Q, E228G, E60Q, G110E, G33C, G70E, H14R, H172P, |
| | H69Y, H78Y, I101T, I141M, I258V, K162Q, K189M, L16M, L197R, L255Q, |
| | L48S, M267T, N131S, M1? |
| Truncations | C272fs*13, K271fs*44, R125*, Q77*, E152*, E217*, Q66*, E39*, R265fs*2, |
| | S279*, A85fs*66, W13*, E188fs*7, E150*, K46fs*3, P133fs*21, R213*, W57*, |
| | F211fs*4, L251fs*30, Y134*, P146*, Q173*, W167*, D90fs*10, E158fs*15, |
| | F207fs*8, N183fs*22, Q107*, L142fs*12, R140*, R186*, T127fs*3, |
| | *291Qext*42, C40fs*5, D145fs*8, E188fs*12, G264fs*21, N192fs*13, |
| | *291Sext*42, A85fs*5, F7fs*27, K41*, Q100*, Q66fs*13, E237*, E71*, |
| | H151fs*3, Q275*, T290fs*3+, *291Yext*42, I199fs*16, L241*, L106*, F9fs*27 |
| Splice Site Mutations | Splice site 791 − 1G > A, splice site 791 − 1G > T, splice site 52 + 2T > C, splice site |
| | 683 − 1G > A, splice site 394 + 1G > A, splice site 630_682 + 272del325, splice site |
| | 682 + 1G > A, splice site 790 + 1G > A, splice site 683 − 2A > C, splice site 53 − |
| | 49_82del79, splice site 790 + 1G > T, splice site 851 − 1G > C, splice site 683 − 1G > T, |
| | splice site 52 + 1G > A, splice site 791 − 1G > C, splice site 394 + 2T > A, splice site |
| | 790 + 1_790 + 4delGTAG, |
| Insertions/Deletions | T203del, R213del, E31_Y32insFTVTVPKDLYVVE, K41_E45 > R, |
| | T290_T290 > ? |

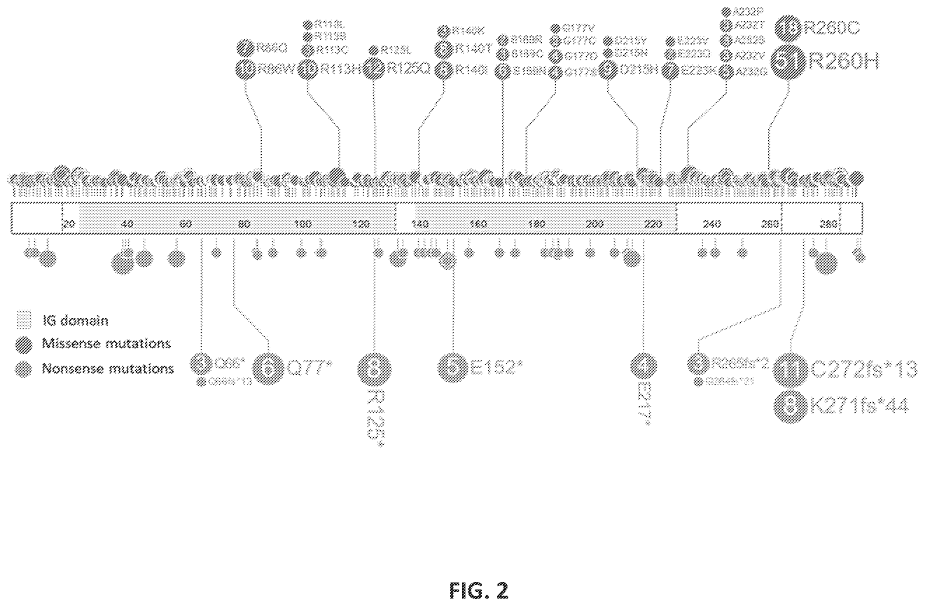

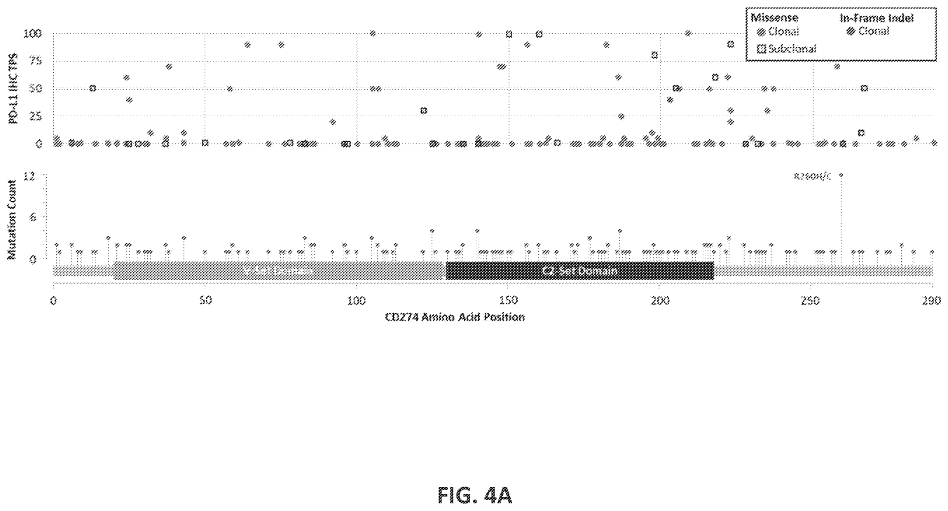

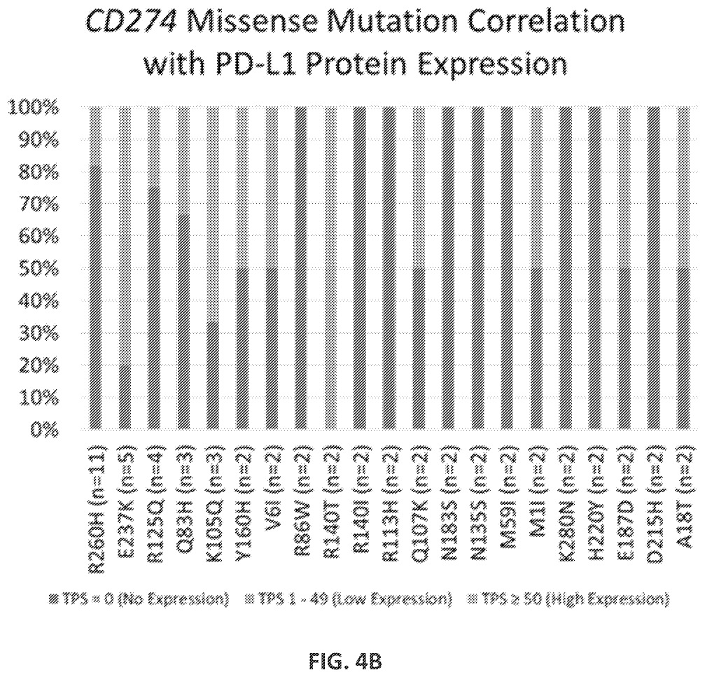

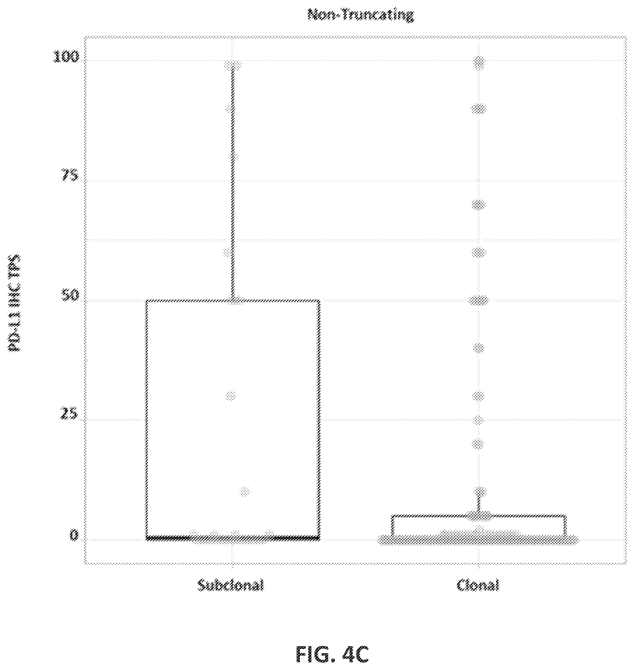

In some embodiments, the one or more CD274 mutations comprise one or more of the mutations provided in any of Tables 1-4 and 6. In some embodiments, the one or more CD274 mutations comprise one or more of the mutations provided herein in Example 1. In some embodiments, the one or more CD274 mutations comprise one or more of the mutations provided herein in FIG. 2 or FIG. 4B. In some embodiments, the one or more CD274 mutations comprise a fusion of a CD274 gene or a portion thereof with another gene (or a portion thereof). In some embodiments, the one or more CD274 mutations comprise a fusion of a CD274 gene, or a portion thereof, with a PLGRKT gene, or a portion thereof (i.e., a CD274-PLGRKT fusion). In some embodiments, the one or more CD274 mutations comprise a CD274 intragenic rearrangement.

In some embodiments, the one or more CD274 mutations are somatic mutations. In some embodiments, the one or more CD274 mutations are germline mutations. Whether a CD274 mutation is a germline mutation or a somatic mutation may be assessed using any suitable method known in the art, such as using a Somatic Germline Zygosity (SGZ) bioinformatics algorithm, see, e.g., Sun et al., PLOS Computational Biology (2018) 14(2):e1005965.

In some embodiments, the one or more CD274 mutations are clonal mutations. In some embodiments, the one or more CD274 mutations are sub-clonal mutations. In some embodiments, the clonality of a CD274 mutation is assessed using any suitable method known in the art. In some embodiments, a CD274 mutation is determined to be a sub-clonal mutation if in a sample, e.g., a sample obtained from an individual having a cancer, such as sample from the cancer or from a tumor, less than 50% of tumor cells comprise or are predicted to have the CD274 mutation, e.g., based on the variant allele fraction (VAF) and/or pathologic tumor cell purity estimates and/or computational tumor cell purity estimates.

In some embodiments, the one or more CD274 mutations comprise one or more missense mutations selected from R260H, R260C, R125Q, R86W, R113H, D215H, R1401, R140T, H233Y, A18T, R86Q, E223K, P24S, M266I, Y112C, S169N, A163V, G245E, E217Q, A232G, D284A, A85V, G177S, K280N, T290A, A52V, E158K, H220Y, K105Q, P235S, Q83H, S184F, R262I, E187Q, E217K, Q139R, K25N, M361, Q173E, E205Q, D61N, F207L, G177D, K129N, D276Y, G119D, N183S, P146L, P43S, R140K, A232T, A232V, E187D, E289D, G95E, M1I, P230S, R265T, A109V, A5V, F9L, Q156H, R262K, R265K, W13C, A232S, P216S, S79N, T148A, D268N, G264E, M101, P227S, R212K, E150K, G110R, K185N, L231V, P146S, S169C, D145Y, E237K, E58K, L16P, L229P, N135S, A132V, G95R, I258M, I274M, R113C, S279L, V2531, E218K, E188K, E223Q, E288Q, F67C, K75T, L241F, Q173H, Q47E, T1791, T201I, V193M, V21L, W57G, Y208C, D122N, D268H, D61Y, E71Q, G119S, G70A, H78R, K178E, L27I, N131I, P235L, Q107K, S169R, T210S, V21A, V2421, A157T, D284N, E164Q, L244M, L249V, Q66E, R2M, V165A, D268G, D26G, E288G, I8K, K62N, L142F, P133L, P133S, P216L, P43Q, T2851, V174G, V1741, V174L, V6I, A85T, C209Y, D103Y, D276N, E39K, E39Q, F257L, I8V, K75N, L106F, M591, R198K, V143A, V147A, V30A, D108N, D122Y, D73N, D90Y, G159C, G177C, H14Y, 138T, L190F, L53P, R238K, T221A, V29M, V68E, V76F, C272Y, D171G, E188V, E71K, H69N, K25R, L92H, T196A, T2211, V242A, A51V, A97V, A98T, E45K, E45Q, F257C, H69R, P161L, P216H, Q139E, Q91H, S80R, T181A, T256S, V23F, V68A, Y160H, D90A, E152Q, E158V, E288K, E31G, F4S, G245A, G245V, G252D, G264V, G70R, 1126F, 1126L, 1199V, 138M, 13T, K162N, K189Q, L197P, L244V, L50V, L94Q, N135D, N183D, N1921, P161S, P24A, Q275R, R113S, R238T, R265G, R82K, S283T, V111L, V253G, V269M, P234S, Q47R, R186G, R198T, R2121, S176G, T1021, T20A, T37K, V174A, V174D, Y28S, A121V, A222V, A246V, D215Y, D49G, D61V, E288D, F211C, G252C, G264R, H240L, 1166V, 165M, K25M, K89N, L16R, L190I, L255R, L53I, M59T, N236D, P227A, P234R, Q100R, Q156E, Q83R, R2K, S1951, T154I, T182P, T22S, V128L, V130L, V174F, Y12F, Y32C, Y56C, C209F, C209S, D171N, D276E, 1206M, 1206T, 1226L, 1226M, 1243V, K136E, M115L, N200S, N204I, N219T, N236I, N96S, P227T, Q100H, Q282R, Q91E, R198G, R262G, R2W, T203S, V1431, V44A, V55F, V68M, Y123H, A51D, A85S, A98V, C250G, C272R, D284Y, D49N, E152K, E158G, E188D, E218Q, G119V, 164V, K124N, K46R, L248S, L287V, M10T, M10V, M36T, M36V, N131K, N138T, N17D, N200D, N219I, N236H, S79G, T194A, T285S, W167C, W57S, Y118F, Y134C, A18S, A222G, A232P, A97T, C114Y, C250Y, D215N, E150G, E31A, F211L, G252S, G264W, H172Y, H240P, 1258F, 164M, 164T, K281N, L190R, L249S, L251F, N131H, N138H, N138K, N35D, P43A, S195R, T127A, T1801, T2021, T202R, T239S, T37A, V231, V29G, D145E, D61H, D73H, E164A, E187V, E237Q, F7L, G159R, H240Y, 1141V, N35K, N96H, N96Y, P230L, P230T, R125L, S184P, S195N, T1811, T203A, T2031, T285A, V143F, V761, A246D, D122E, D145H, E187K, E205K, E223V, E228V, E31K, F2111, F259L, G177V, G273D, H151L, H69L, 1126S, 1166L, 1243T, 154L, K189E, K271N, K280Q, L190V, L231M, L244P, L255M, L50M, L53R, L88F, M115T, M2671, N204K, N35H, N63Y, P234F, P43L, R186K, R213K, R2G, R821, A157S, A51S, D103N, D26N, D90E, E158Q, E58G, F671, H151R, H172Q, H220D, 13M, K162R, K185E, K263E, L142W, L261F, N183H, P234T, Q156K, R113L, R186T, R212T, R2651, S149Y, T154S, T285P, V165L, Y81H, A132D, A254G, A5D, D103H, D145N, D26Y, D276H, D61E, E188Q, E228G, E60Q, G110E, G33C, G70E, H14R, H172P, H69Y, H78Y, I101T, I141M, 1258V, K162Q, K189M, L16M, L197R, L255Q, L48S, M267T, M1?, or N131S. Alternatively or additionally, in some embodiments, the one or more CD274 mutations comprise one or more truncating mutations selected from C272fs*13, K271fs*44, R125*, Q77*, E152*, E217*, Q66*, E39*, R265fs*2, S279*, A85fs*66, W13*, E188fs*7, E150*, K46fs*3, P133fs*21, R213*, W57*, F211fs*4, L251fs*30, Y134*, P146*, Q173*, W167*, D90fs*10, E158fs*15, F207fs*8, N183fs*22, Q107*, L142fs*12, R140*, R186*, T127fs*3, *291Qext*42, C40fs*5, D145fs*8, E188fs*12, G264fs*21, N192fs*13, *291Sext*42, A85fs*5, F7fs*27, K41*, Q100*, Q66fs*13, E237*, E71*, H151fs*3, Q275*, T290fs*3+,*291Yext*42, I199fs*16, L241*, L106*, or F9fs*27. Alternatively or additionally, in some embodiments, the one or more CD274 mutations comprise one or more splice site mutations selected from splice site 791-1G>A, splice site 791-1G>T, splice site 52+2T>C, splice site 683-1G>A, splice site 394+1G>A, splice site 630_682+272del325, splice site 682+1G>A, splice site 790+1G>A, splice site 683-2A>C, splice site 53-49_82del79, splice site 790+1G>T, splice site 851-1G>C, splice site 683-1G>T, splice site 52+1G>A, splice site 791-1G>C, splice site 394+2T>A, or splice site 790+1_790+4delGTAG. Alternatively or additionally, in some embodiments, the one or more CD274 mutations comprise one or more insertion/deletions selected from T203del, R213del, E31_Y32insFTVTVPKDLYVVE, K41_E45>R, or T290_T290>?.

In some embodiments, the one or more mutations in a CD274 gene result in low expression of a PD-L1 protein, no expression of a PD-L1 protein, or high expression of a PD-L1 protein, e.g., in a cancer, cancer cell, tumor or tumor cell comprising the one or more mutations. In some embodiments, PD-L1 protein expression is assessed using an immunohistochemistry assay in sample obtained from an individual, e.g., an individual having a cancer. In some embodiments, PD-L1 protein expression is assessed in tumor cells. In some embodiments, a low expression of a PD-L1 protein in the cancer (e.g., in sample from the cancer or from a tumor) is assessed based on a tumor proportion score (TPS) of between 1% and 49%. In some embodiments, a high expression of a PD-L1 protein in the cancer (e.g., in sample from the cancer or from a tumor) is assessed based on a tumor proportion score (TPS) of 50% or greater. In some embodiments, the one or more mutations in a CD274 gene comprise a truncating mutation. In some embodiments, the truncating mutation is a clonal or sub-clonal mutation. Further information about PD-L1 protein expression and methods of assessing PD-L1 protein expression is provided herein infra.

Exemplary and non-limiting CD274 gene mutations that result in low expression of a PD-L1 protein include those listed in Table 2.

TABLE 2
CD274 variants associated with low PD-L1 expression
(TPS score of between 1% and 49%).

| CD274 Mutation | PD-L1 TPS |
|---|---|
| A18T | 1 |
| A254G | 1 |
| D61Y | 1 |
| E187Q | 1 |
| F9L | 1 |
| H78R | 1 |
| I166L | 1 |
| K162Q | 1 |
| L50V | 1 |
| P43A | 1 |
| P43Q | 1 |
| Q83H | 1 |
| R125Q | 1 |
| R260H | 1 |
| T290A | 1 |
| V21L | 1 |
| V6I | 1 |
| E152* | 1 |
| K271fs*44 | 1 |
| V242I | 1 |
| P216H | 2 |
| A109V | 5 |
| A163V | 5 |
| D284N | 5 |
| E188Q | 5 |
| I199V | 5 |
| M1I | 5 |
| P230S | 5 |
| R140T | 5 |
| S195R | 5 |
| T181I | 5 |
| T37K | 5 |
| R125* | 5 |
| S279* | 5 |
| L197P | 10 |
| M266I | 10 |
| P43L | 10 |
| Y32C | 10 |
| E223K | 20 |
| L92H | 20 |
| E217* | 20 |
| E187D | 25 |
| D122N | 30 |
| E223K | 30 |
| P235L | 30 |
| splice site 790 + 1_790 + 4delGTAG | 30 |
| T203del | 40 |
| K25R | 40 |

Exemplary and non-limiting CD274 gene mutations that result in no expression of a PD-L1 protein include those listed in Table 3.

TABLE 3
CD274 variants associated with no PD-L1
expression (TPS score of less than 1%).

| CD274 Mutation | PD-L1 TPS |
|---|---|
| A157S | 0 |
| A18S | 0 |
| A18T | 0 |
| A232S | 0 |
| A85S | 0 |
| A85V | 0 |
| A97V | 0 |
| C272Y | 0 |
| D145Y | 0 |
| D171G | 0 |
| D171N | 0 |
| D215H | 0 |
| D276E | 0 |
| E187D | 0 |
| E187V | 0 |
| E217K | 0 |
| E217Q | 0 |
| E228G | 0 |
| E228V | 0 |
| E237K | 0 |
| E71Q | 0 |
| F211C | 0 |
| F257L | 0 |
| G110E | 0 |
| G177D | 0 |
| G177S | 0 |
| G177V | 0 |
| G245V | 0 |
| G252S | 0 |
| G264W | 0 |
| H14Y | 0 |
| H151L | 0 |
| H172Y | 0 |
| H220Y | 0 |
| H233Y | 0 |
| I126S | 0 |
| I243T | 0 |
| I8K | 0 |
| K105Q | 0 |
| K178E | 0 |
| K25N | 0 |
| K280N | 0 |
| L142F | 0 |
| L190V | 0 |
| M1I | 0 |
| M59I | 0 |
| N135S | 0 |
| N183S | 0 |
| N200D | 0 |
| N96S | 0 |
| N96Y | 0 |
| P133S | 0 |
| P146S | 0 |
| P24S | 0 |
| Q100R | 0 |
| Q107K | 0 |
| Q156H | 0 |
| Q173E | 0 |
| Q173H | 0 |
| Q275R | 0 |
| Q83H | 0 |
| R113H | 0 |
| R125Q | 0 |
| R140I | 0 |
| R198T | 0 |
| R212K | 0 |
| R260C | 0 |
| R260H | 0 |
| R2M | 0 |
| R82I | 0 |
| R86W | 0 |
| T180I | 0 |
| T196A | 0 |
| T201I | 0 |
| T37A | 0 |
| V130L | 0 |
| V143A | 0 |
| V21A | 0 |
| V253I | 0 |
| V30A | 0 |
| V6I | 0 |
| V76F | 0 |
| W57G | 0 |
| Y112C | 0 |
| Y134C | 0 |
| Y160H | 0 |
| Y28S | 0 |
| Y81H | 0 |
| *291Yext*42 | 0 |
| C272fs*13 | 0 |
| D90fs*10 | 0 |
| E152* | 0 |
| E188fs*12 | 0 |
| F7fs*27 | 0 |
| K271fs*44 | 0 |
| K41* | 0 |
| P133fs*21 | 0 |
| Q107* | 0 |
| Q77* | 0 |
| R125* | 0 |
| R265fs*2 | 0 |
| Y134* | 0 |
| splice site 683 − 1G > A | 0 |
| splice site 790 + 1G > T | 0 |
| splice site 791 − 1G > A | 0 |
| splice site 791 − 1G > T | 0 |
| E31_Y32insFTVTVPKDLYVVE | 0 |
| I141M, F9L | 0 |
| K189M, E188K | 0 |

Exemplary and non-limiting CD274 gene mutations that result in high expression of a PD-L1 protein include those listed in Table 4.

TABLE 4
CD274 variants associated with high PD-L1
expression (TPS score of 50% or more).

| CD274 Mutation | PD-L1 TPS |
|---|---|
| E205Q | 50 |
| E237K | 50 |
| E58K | 50 |
| I206M | 50 |
| K105Q | 50 |
| M267I | 50 |
| P216S | 50 |
| P234R | 50 |
| Q107K | 50 |
| W13C | 50 |
| A222V | 60 |
| E218K | 60 |
| P24A | 60 |
| R186T | 60 |
| I258V | 70 |
| I38M | 70 |
| T148A | 70 |
| V147A | 70 |
| Q66fs*13 | 70 |
| splice site 53 − 49_82del79 | 70 |
| R198K | 80 |
| E150* | 80 |
| E223K | 90 |
| I64T | 90 |
| K75T | 90 |
| Q156K | 90 |
| T182P | 90 |
| R140T | 99 |
| Y160H | 99 |
| E150G | 99 |
| C209S | 100 |
| K105Q | 100 |

In some embodiments, the one or more mutations in a CD274 gene resulting in low expression of a PD-L1 protein comprise one or more of A18T, A254G, D61Y, E187Q, F9L, H78R, I166L, K162Q, L50V, P43A, P43Q, Q83H, R125Q, R260H, T290A, V21L, V6I, E152*, K271fs*44, V242I, P216H, A109V, A163V, D284N, E188Q, I199V, M1I, P230S, R140T, S195R, T181I, T37K, R125*, S279*, L197P, M266I, P43L, Y32C, E223K, L92H, E217*, E187D, D122N, E223K, P235L, splice site 790+1_790+4delGTAG, T203del, or K25R. In some embodiments, the one or more mutations in a CD274 gene resulting in no expression of a PD-L1 protein comprise one or more of A157S, A18S, A18T, A232S, A85S, A85V, A97V, C272Y, D145Y, D171G, D171N, D215H, D276E, E187D, E187V, E217K, E217Q, E228G, E228V, E237K, E71Q, F211C, F257L, GI1E, G177D, G177S, G177V, G245V, G252S, G264W, H14Y, H151L, H172Y, H220Y, H233Y, I126S, I243T, 18K, K105Q, K178E, K25N, K280N, L142F, L190V, M1I, M59I, N135S, N183S, N200D, N96S, N96Y, P133S, P146S, P24S, Q100R, Q107K, Q156H, Q173E, Q173H, Q275R, Q83H, R113H, R125Q, R140I, R198T, R212K, R260C, R260H, R2M, R821, R86W, T180I, T196A, T201I, T37A, V130L, V143A, V21A, V2531, V30A, V6I, V76F, W57G, Y112C, Y134C, Y160H, Y28S, Y81H, *291Yext*42, C272fs*13, D90fs*10, E152*, E188fs*12, F7fs*27, K271fs*44, K41*, P133fs*21, Q107*, Q77*, R125*, R265fs*2, Y134*, splice site 683-1G>A, splice site 790+1G>T, splice site 791-1G>A, splice site 791-1G>T, E31_Y32insFTVTVPKDLYVVE, I141M, F9L, K189M, or E188K. In some embodiments, the one or more mutations in a CD274 gene resulting in high expression of a PD-L1 protein comprise one or more of E205Q, E237K, E58K, I206M, K105Q, M267I, P216S, P234R, Q107K, W13C, A222V, E218K, P24A, R186T, 1258V, I38M, T148A, V147A, Q66fs*13, splice site 53-49_82del79, R198K, E150*, E223K, I64T, K75T, Q156K, T182P, R140T, Y160H, E150G, C209S, or K105Q. In some embodiments, the one or more mutations in a CD274 gene comprise a truncating mutation. In some embodiments, the truncating mutation is a clonal or sub-clonal mutation.

In some embodiments, detection of one or more mutations in a CD274 gene as described herein is performed in vitro.

### Cancers of the Disclosure

In some embodiments, a cancer of the present disclosure is a carcinoma, a sarcoma, a lymphoma, a leukemia, a myeloma, a germ cell cancer, or a blastoma. In some embodiments, the cancer is a solid tumor. In some embodiments, the cancer is a hematologic malignancy.

Exemplary and non-limiting examples of cancers that may comprise one or more CD274 mutations of the disclosure include a lung cancer (e.g., a non-small cell lung cancer (NSCLC)), a kidney cancer (e.g., a kidney urothelial carcinoma), a bladder cancer (e.g., a bladder urothelial (transitional cell) carcinoma), a breast cancer, a colorectal cancer (e.g., a colon adenocarcinoma), an ovarian cancer, a pancreatic cancer, a gastric carcinoma, an esophageal cancer, a mesothelioma, a melanoma (e.g., a skin melanoma), a head and neck cancer (e.g., a head and neck squamous cell carcinoma (HNSCC)), a thyroid cancer, a sarcoma (e.g., a soft-tissue sarcoma, a fibrosarcoma, a myxosarcoma, a liposarcoma, an osteogenic sarcoma, an osteosarcoma, a chondrosarcoma, an angiosarcoma, an endotheliosarcoma, a lymphangiosarcoma, a lymphangioendotheliosarcoma, a leiomyosarcoma, or a rhabdomyosarcoma), a prostate cancer, a glioblastoma, a cervical cancer, a thymic carcinoma, a leukemia (e.g., an acute lymphocytic leukemia (ALL), an acute myelocytic leukemia (AML), a chronic myelocytic leukemia (CML), a chronic eosinophilic leukemia, or a chronic lymphocytic leukemia (CLL)), a lymphoma (e.g., a Hodgkin lymphoma or a non-Hodgkin lymphoma (NHL)), a myeloma (e.g., a multiple myeloma (MM)), a mycoses fungoides, a merkel cell cancer, a hematologic malignancy, a cancer of hematological tissues, a B cell cancer, a bronchus cancer, a stomach cancer, a brain or central nervous system cancer, a peripheral nervous system cancer, a uterine or endometrial cancer, a cancer of the oral cavity or pharynx, a liver cancer, a testicular cancer, a biliary tract cancer, a small bowel or appendix cancer, a salivary gland cancer, an adrenal gland cancer, an adenocarcinoma, an inflammatory myofibroblastic tumor, a gastrointestinal stromal tumor (GIST), a colon cancer, a myelodysplastic syndrome (MDS), a myeloproliferative disorder (MPD), a polycythemia Vera, a chordoma, a synovioma, an Ewing's tumor, a squamous cell carcinoma, a basal cell carcinoma, an adenocarcinoma, a sweat gland carcinoma, a sebaceous gland carcinoma, a papillary carcinoma, a papillary adenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilms' tumor, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodendroglioma, a meningioma, a neuroblastoma, a retinoblastoma, a follicular lymphoma, a diffuse large B-cell lymphoma, a mantle cell lymphoma, a hepatocellular carcinoma, a thyroid cancer, a small cell cancer, an essential thrombocythemia, an agnogenic myeloid metaplasia, a hypereosinophilic syndrome, a systemic mastocytosis, a familiar hypereosinophilia, a neuroendocrine cancer, or a carcinoid tumor.

Additional exemplary and non-limiting examples of cancers that may comprise one or more CD274 mutations of the disclosure include Acute b-lymphoblastic leukemia-lymphoma (B-ALL), Acute leukemia (NOS), Acute lymphoblastic leukemia-lymphoma (ALL) (NOS), Acute myeloid leukemia, Acute myeloid leukemia (AML) (NOS), Acute t-lymphoblastic leukemia-lymphoma (T-ALL), Adrenal gland cortical carcinoma, Adrenal gland neuroblastoma, Ampullary adenocarcinoma, Anus melanoma, Anus squamous cell carcinoma (SCC), Appendix adenocarcinoma, Appendix goblet cell carcinoid (GCC), Appendix mucinous neoplasm, B-cell neoplasm (NOS), Bile duct adenocarcinoma, Bladder adenocarcinoma, Bladder carcinoma (NOS), Bladder small cell carcinoma, Bladder squamous cell carcinoma (SCC), Bladder urothelial (transitional cell) carcinoma, Bladder urothelial carcinoma, Bone chondrosarcoma, Bone chordoma, Bone marrow failure (NOS), Bone osteosarcoma, Brain anaplastic astrocytoma, Brain anaplastic oligodendroglioma, Brain astrocytoma, Brain astrocytoma pilocytic, Brain ependymoma, Brain ganglioglioma, Brain glioblastoma (GBM), Brain glioma, Brain glioma (NOS), Brain gliosarcoma, Brain medulloblastoma, Brain meningioma, Brain oligodendroglioma, Breast angiosarcoma, Breast carcinoma, Breast carcinoma (NOS), Breast invasive ductal carcinoma (IDC), Breast invasive lobular carcinoma (ILC), Breast metaplastic carcinoma, Cervix adenocarcinoma, Cervix neuroendocrine carcinoma, Cervix squamous cell carcinoma (SCC), Chronic lymphocytic leukemia/small lymphocytic lymphoma (CLL/SLL), Chronic myelogenous leukemia (CML), Colon adenocarcinoma, Colon adenocarcinoma (CRC), Colon neuroendocrine carcinoma, Diffuse large B-cell lymphoma (DLBCL), Diffuse large B-cell lymphoma (DLBCL) (NOS), Duodenum adenocarcinoma, Esophagus adenocarcinoma, Esophagus carcinoma (NOS), Esophagus neuroendocrine carcinoma, Esophagus squamous cell carcinoma (SCC), Eye intraocular melanoma, Fallopian tube adenocarcinoma, Fallopian tube carcinosarcoma, Fallopian tube serous carcinoma, Follicular lymphoma, Gallbladder adenocarcinoma, Gallbladder adenocarcinoma, Gastroesophageal junction adenocarcinoma, Head and neck adenoid cystic carcinoma, Head and neck carcinoma (NOS), Head and neck melanoma, Head and neck squamous cell carcinoma (HNSCC), Kidney carcinoma (NOS), Kidney clear cell carcinoma, Kidney renal cell carcinoma, Kidney renal papillary carcinoma, Kidney sarcomatoid carcinoma, Kidney urothelial carcinoma, Kidney Wilms tumor, Langerhans cell histiocytosis (LCH), Liver cholangiocarcinoma, Liver hepatocellular carcinoma (HCC), Lung adenocarcinoma, Lung adenoid cystic carcinoma, Lung adenosquamous carcinoma, Lung atypical carcinoid, Lung large cell carcinoma, Lung large cell neuroendocrine carcinoma, Lung non-small cell lung carcinoma, Lung non-small cell lung carcinoma (NSCLC) (NOS), Lung sarcomatoid carcinoma, Lung small cell undifferentiated carcinoma, Lung squamous cell carcinoma (SCC), Lymphoplasmacytic lymphoma-Waldenstroms macroglobulinemia, Mantle cell lymphoma, Multiple myeloma, Myelodysplastic syndrome, Myelodysplastic syndrome (MDS) (NOS), Myelodysplastic-myeloproliferative neoplasm (MDS-MPN) (NOS), Myeloproliferative neoplasm, Myeloproliferative neoplasm (MPN) (NOS), Nasopharynx and paranasal sinuses squamous cell carcinoma (SCC), Nasopharynx and paranasal sinuses undifferentiated carcinoma, Neuroblastoma, Non-hodgkin lymphoma (NOS), Ovary carcinoma mixed histology, Ovary carcinosarcoma, Ovary clear cell carcinoma, Ovary endometrioid adenocarcinoma, Ovary epithelial carcinoma, Ovary granulosa cell tumor, Ovary high grade serous carcinoma, Ovary low grade serous carcinoma, Ovary mucinous carcinoma, Ovary serous carcinoma, Pancreas acinar cell carcinoma, Pancreas adenosquamous carcinoma, Pancreas carcinoma (NOS), Pancreas ductal adenocarcinoma, Pancreas islet cell tumor, Pancreatobiliary carcinoma, Paraganglioma, Penis squamous cell carcinoma (SCC), Peritoneum adenocarcinoma, Peritoneum mesothelioma, Peritoneum serous carcinoma, Pleura mesothelioma, Prostate acinar adenocarcinoma, Prostate ductal adenocarcinoma, Prostate neuroendocrine carcinoma, Prostate undifferentiated carcinoma, Prostate undifferentiated carcinoma, Rectum adenocarcinoma, Rectum adenocarcinoma (CRC), Rectum squamous cell carcinoma (SCC), Rhabdomyosarcoma (NOS), Salivary gland acinic cell tumor, Salivary gland adenocarcinoma, Salivary gland adenoid cystic carcinoma, Salivary gland carcinoma (NOS), Salivary gland duct carcinoma, Salivary gland mucoepidermoid carcinoma, Skin adnexal carcinoma, Skin adnexal carcinoma, Skin basal cell carcinoma, Skin melanoma, Skin merkel cell carcinoma, Skin squamous cell carcinoma (SCC), Small intestine adenocarcinoma, Small intestine carcinoid, Small intestine gist, Small intestine neuroendocrine carcinoma, Soft tissue angiosarcoma, Soft tissue chondrosarcoma, Soft tissue desmoplastic small round cell tumor, Soft tissue ewing sarcoma, Soft tissue fibromatosis, Soft tissue fibrosarcoma, Soft tissue leiomyosarcoma, Soft tissue liposarcoma, Soft tissue malignant peripheral nerve sheath tumor (MPNST), Soft tissue myxofibrosarcoma, Soft tissue rhabdomyosarcoma alveolar, Soft tissue rhabdomyosarcoma embryonal, Soft tissue sarcoma, Soft tissue sarcoma (NOS), Soft tissue sarcoma undifferentiated, Soft tissue solitary fibrous tumor, Soft tissue synovial sarcoma, Stomach adenocarcinoma (NOS), Stomach adenocarcinoma diffuse type, Stomach adenocarcinoma intestinal type, Stomach gist, T-cell neoplasm (NOS), Testis germ cell tumor (non-seminoma), Thymus carcinoma, Thymus thymoma (NOS), Thyroid anaplastic carcinoma, Thyroid carcinoma, Thyroid carcinoma (NOS), Thyroid follicular carcinoma, Thyroid medullary carcinoma, Thyroid papillary carcinoma, Unknown primary (NOS), Unknown primary adenocarcinoma, Unknown primary adenoid cystic carcinoma, Unknown primary carcinoid, Unknown primary carcinoma, Unknown primary carcinoma (CUP) (NOS), Unknown primary gist, Unknown primary leiomyosarcoma, Unknown primary melanoma, Unknown primary neuroendocrine carcinoma, Unknown primary neuroendocrine tumor, Unknown primary sarcoma, Unknown primary sarcomatoid carcinoma, Unknown primary serous carcinoma, Unknown primary squamous cell carcinoma (SCC), Unknown primary undifferentiated neuroendocrine carcinoma, Unknown primary undifferentiated small cell carcinoma, Unknown primary urothelial carcinoma, Ureter urothelial carcinoma, Uterine endometrial adenocarcinoma, Uterus carcinosarcoma, Uterus endometrial adenocarcinoma, Uterus endometrial adenocarcinoma (NOS), Uterus endometrial adenocarcinoma clear cell, Uterus endometrial adenocarcinoma endometrioid, Uterus endometrial adenocarcinoma mixed histology, Uterus endometrial adenocarcinoma papillary serous, Uterus endometrial stromal sarcoma, Uterus leiomyosarcoma, Uterus sarcoma (NOS), Vagina squamous cell carcinoma (SCC), and Vulva squamous cell carcinoma (SCC).

Further exemplary and non-limiting examples of cancers that may comprise one or more CD274 mutations of the disclosure include those listed in Table 5.

TABLE 5
Cancer types.
Cancer types

| | |
|---|---|
| Lung adenocarcinoma | Colon adenocarcinoma (CRC) |
| Breast carcinoma (NOS) | Unknown primary adenocarcinoma |
| Pancreas ductal adenocarcinoma | Brain glioblastoma (GBM) |
| Breast invasive ductal carcinoma (IDC) | Rectum adenocarcinoma (CRC) |
| Prostate acinar adenocarcinoma | Liver cholangiocarcinoma |
| Lung squamous cell carcinoma (SCC) | Bladder urothelial (transitional cell) carcinoma |
| Ovary serous carcinoma | Lung small cell undifferentiated carcinoma |
| Lung non-small cell lung carcinoma (NSCLC) | Multiple myeloma |
| (NOS) | |
| Esophagus adenocarcinoma | Acute myeloid leukemia (AML) (NOS) |
| Stomach adenocarcinoma (NOS) | Pancreatobiliary carcinoma |
| Head and neck squamous cell carcinoma | Uterus endometrial adenocarcinoma |
| (HNSCC) | endometrioid |
| Unknown primary carcinoma (CUP) (NOS) | Soft tissue sarcoma (NOS) |
| Skin melanoma | Uterus endometrial adenocarcinoma papillary |
| | serous |
| Unknown primary melanoma | Ovary high grade serous carcinoma |
| Uterus endometrial adenocarcinoma (NOS) | Kidney renal cell carcinoma |
| Myelodysplastic syndrome (MDS) (NOS) | Pancreas carcinoma (NOS) |
| Ovary epithelial carcinoma | Unknown primary squamous cell carcinoma |
| | (SCC) |
| Gastroesophageal junction adenocarcinoma | Gallbladder adenocarcinoma |
| Breast invasive lobular carcinoma (ILC) | Prostate ductal adenocarcinoma |
| Kidney clear cell carcinoma | Kidney Wilms tumor |
| Unknown primary undifferentiated | Rectum squamous cell carcinoma (SCC) |
| neuroendocrine carcinoma | |
| Liver hepatocellular carcinoma (HCC) | Esophagus squamous cell carcinoma (SCC) |
| Fallopian tube serous carcinoma | Soft tissue liposarcoma |
| Myeloproliferative neoplasm (MPN) (NOS) | Thyroid papillary carcinoma |
| Cervix squamous cell carcinoma (SCC) | Pleura mesothelioma |
| Soft tissue leiomyosarcoma | Diffuse large b-cell lymphoma (DLBCL) (NOS) |
| Uterus carcinosarcoma | Uterus leiomyosarcoma |
| Appendix adenocarcinoma | Stomach adenocarcinoma intestinal type |
| Unknown primary (NOS) | Bone osteosarcoma |
| Lung large cell neuroendocrine carcinoma | Duodenum adenocarcinoma |
| Brain glioma (NOS) | Acute b-lymphoblastic leukemia-lymphoma (B- |
| | ALL) |
| Ovary clear cell carcinoma | Chronic lymphocytic leukemia/small |
| | lymphocytic lymphoma (CLL/SLL) |
| Brain anaplastic astrocytoma | Unknown primary gist |
| Skin squamous cell carcinoma (SCC) | Ovary endometrioid adenocarcinoma |
| Anus squamous cell carcinoma (SCC) | Acute lymphoblastic leukemia-lymphoma (ALL) |
| | (NOS) |
| Peritoneum serous carcinoma | Ovary carcinosarcoma |
| Brain meningioma | Unknown primary serous carcinoma |
| Pancreas islet cell tumor | Adrenal gland cortical carcinoma |
| Brain astrocytoma | Brain oligodendroglioma |
| Cervix adenocarcinoma | Ampullary adenocarcinoma |
| Bile duct adenocarcinoma | Thyroid carcinoma (NOS) |
| Kidney urothelial carcinoma | Thyroid anaplastic carcinoma |
| Small intestine adenocarcinoma | Head and neck adenoid cystic carcinoma |
| Unknown primary urothelial carcinoma | Stomach gist |
| Soft tissue synovial sarcoma | Neuroblastoma |
| Salivary gland carcinoma (NOS) | Ovary granulosa cell tumor |
| Lung adenosquamous carcinoma | Vulva squamous cell carcinoma (SCC) |
| Salivary gland adenoid cystic carcinoma | Skin merkel cell carcinoma |
| Soft tissue ewing sarcoma | Stomach adenocarcinoma diffuse type |
| Soft tissue angiosarcoma | Prostate neuroendocrine carcinoma |
| Lung sarcomatoid carcinoma | Breast metaplastic carcinoma |
| Ureter urothelial carcinoma | Thymus carcinoma |
| Soft tissue sarcoma undifferentiated | Kidney renal papillary carcinoma |
| Unknown primary neuroendocrine tumor | Brain astrocytoma pilocytic |
| Colon neuroendocrine carcinoma | Uterus endometrial adenocarcinoma clear cell |
| Unknown primary undifferentiated small cell | Acute leukemia (NOS) |
| carcinoma | |
| Soft tissue malignant peripheral nerve sheath | Brain ependymoma |
| tumor (MPNST) | |
| Eye intraocular melanoma | Thymus thymoma (NOS) |
| Salivary gland adenocarcinoma | Mantle cell lymphoma |
| Brain medulloblastoma | T-cell neoplasm (NOS) |
| Peritoneum mesothelioma | Skin basal cell carcinoma |
| Follicular lymphoma | B-cell neoplasm (NOS) |
| Thyroid medullary carcinoma | Bone chondrosarcoma |
| Bladder carcinoma (NOS) | Penis squamous cell carcinoma (SCC) |
| Esophagus carcinoma (NOS) | Soft tissue myxofibrosarcoma |
| Small intestine gist | Prostate undifferentiated carcinoma |
| Myelodysplastic-myeloproliferative neoplasm | Bladder adenocarcinoma |
| (MDS-MPN) (NOS) | |
| Rhabdomyosarcoma (NOS) | Nasopharynx and paranasal sinuses |
| | undifferentiated carcinoma |
| Soft tissue fibromatosis | Unknown primary leiomyosarcoma |
| Uterus endometrial stromal sarcoma | Acute t-lymphoblastic leukemia-lymphoma (T- |
| | ALL) |
| Soft tissue chondrosarcoma | Adrenal gland neuroblastoma |
| Bone chordoma | Chronic myelogenous leukemia (CML) |
| Lung large cell carcinoma | Appendix goblet cell carcinoid (GCC) |
| Thyroid follicular carcinoma | Unknown primary adenoid cystic carcinoma |
| Lung atypical carcinoid | Unknown primary sarcomatoid carcinoma |
| Uterus sarcoma (NOS) | Salivary gland duct carcinoma |
| Skin adnexal carcinoma | Ovary carcinoma mixed histology |
| Soft tissue solitary fibrous tumor | Salivary gland acinic cell tumor |
| Appendix mucinous neoplasm | Uterus endometrial adenocarcinoma mixed |
| | histology |
| Ovary mucinous carcinoma | Testis germ cell tumor (non-seminoma) |
| Bladder squamous cell carcinoma (SCC) | Soft tissue desmoplastic small round cell tumor |
| Kidney carcinoma (NOS) | Anus melanoma |
| Pancreas acinar cell carcinoma | Peritoneum adenocarcinoma |
| Unknown primary carcinoid | Brain gliosarcoma |
| Head and neck melanoma | Breast angiosarcoma |
| Vagina squamous cell carcinoma (SCC) | Langerhans cell histiocytosis (LCH) |
| Nasopharynx and paranasal sinuses squamous | Brain anaplastic oligodendroglioma |
| cell carcinoma (SCC) | |
| Small intestine neuroendocrine carcinoma | Non-hodgkin lymphoma (NOS) |
| Kidney sarcomatoid carcinoma | Brain ganglioglioma |
| Head and neck carcinoma (NOS) | Bladder small cell carcinoma |
| Fallopian tube adenocarcinoma | Esophagus neuroendocrine carcinoma |
| Bone marrow failure (NOS) | Lymphoplasmacytic lymphoma-Waldenstroms |
| | macroglobulinemia |
| Ovary low grade serous carcinoma | Cervix neuroendocrine carcinoma |
| Soft tissue fibrosarcoma | Salivary gland mucoepidermoid carcinoma |
| Small intestine carcinoid | Paraganglioma |
| Lung adenoid cystic carcinoma | Soft tissue rhabdomyosarcoma embryonal |
| Soft tissue rhabdomyosarcoma alveolar | Diffuse large B-cell lymphoma (DLBCL) |
| Uterine endometrial adenocarcinoma | Uterus endometrial adenocarcinoma |
| | endometrioid |
| Bladder urothelial carcinoma | Lung non-small cell lung carcinoma |
| Breast carcinoma | Soft tissue sarcoma |
| Unknown primary neuroendocrine carcinoma | Unknown primary carcinoma |
| Myeloproliferative neoplasm | Brain glioma |
| Acute myeloid leukemia | Myelodysplastic syndrome |

In some embodiments, the cancer is diffuse large B-cell lymphoma, cutaneous squamous cell carcinoma, endometrial adenocarcinoma, unknown primary melanoma, or cutaneous melanoma.

In some embodiments, the cancer is a skin cancer. In some embodiments, the cancer comprises a tumor mutational burden (TMB) of ≥10 mutations/Megabase (mut/Mb). In some embodiments, the cancer, such as a skin cancer, comprises a high TMB, e.g., comprises a TMB of at least about 100 mut/Mb, at least about 110 mut/Mb, at least about 120 mut/Mb, at least about 130 mut/Mb, at least about 140 mut/Mb, at least about 150 mut/Mb, or more. In some embodiments, the skin cancer is cutaneous squamous cell carcinoma, cutaneous melanoma, or unknown primary melanoma. Methods of assessing TMB are known in the art and described infra.

In some embodiments, the cancer comprises a high microsatellite instability status (MSI). In some embodiments, the cancer comprises a low microsatellite instability status (MSI). In some embodiments, the cancer is a non-serous endometrial adenocarcinoma, e.g., comprising high MSI (MSI-H). In some embodiments, the cancer is microsatellite stable. Any suitable method for assessing MSI or microsatellite stability may be used, including for example and without limitation, next generation sequencing (see, e.g., Hempelmann et al., J Immunother Cancer (2018) 6(1):29), Fluorescent multiplex PCR and capillary electrophoresis (see, e.g., Arulananda et al., J Thorac Oncol (2018) 13(10):1588-94), immunohistochemistry (see, e.g., Cheah et al., Malays J Pathol (2019) 41(2):91-100), or single-molecule molecular inversion probes (smMIPs, see, e.g., Waalkes et al., Clin Chem (2018) 64(6):950-8). In some embodiments, MSI is assessed based on DNA sequencing (e.g., next generation sequencing) of up to about 114 loci.

In some embodiments, a cancer of the disclosure is a metastatic cancer.

In some embodiments, the cancer of the disclosure is a cancer listed in Table 6 and comprises the corresponding CD2′74 mutations listed in Table 6.

TABLE 6
Cancer type and associated CD274 mutations.

| | |
|---|---|
| CD274 Mutation | Cancer |

Missense Mutations

| | |
|---|---|
| A109V | Pancreas ductal adenocarcinoma |
| A157S | Unknown primary adenocarcinoma |
| A163V | Soft tissue angiosarcoma |
| A18S | Colon adenocarcinoma |
| A18T | Lung adenocarcinoma |
| A18T | Unknown primary carcinoma |
| A222V | Bile duct adenocarcinoma |
| A232S | Prostate acinar adenocarcinoma |
| A254G | Ovary serous carcinoma |
| A85S | Liver hepatocellular carcinoma |
| A85V | Uterus endometrial adenocarcinoma endometrioid |
| A97V | Skin squamous cell carcinoma |
| C209S | Lung adenocarcinoma |
| C272Y | Colon adenocarcinoma |
| D122N | Uterus endometrial adenocarcinoma mixed histology |
| D145Y | Thyroid anaplastic carcinoma |
| D171G | Rectum adenocarcinoma |
| D171N | Head and neck squamous cell carcinoma |
| D215H | Colon adenocarcinoma |
| D215H | Pancreas adenosquamous carcinoma |
| D276E | Ovary serous carcinoma |
| D284N | Skin melanoma |
| D61Y | Liver hepatocellular carcinoma |
| E187D | Unknown primary squamous cell carcinoma |
| E187D | Uterus endometrial adenocarcinoma endometrioid |
| E187Q | Lung adenocarcinoma |
| E187V | Lung adenocarcinoma |
| E188Q | Lung adenocarcinoma |
| E205Q | Unknown primary adenocarcinoma |
| E217K | Pancreas ductal adenocarcinoma |
| E217Q | Lung squamous cell carcinoma |
| E218K | Lung adenocarcinoma |
| E223K | Lung adenocarcinoma |
| E223K | Lung adenocarcinoma |
| E223K | Unknown primary squamous cell carcinoma |
| E228G | Colon adenocarcinoma |
| E228V | Colon adenocarcinoma |
| E237K | Lung adenocarcinoma |
| E237K | Breast invasive ductal carcinoma |
| E58K | Skin melanoma |
| E71Q | Lung adenocarcinoma |
| F211C | Colon adenocarcinoma |
| F257L | Uterus endometrial adenocarcinoma mixed histology |
| F9L | Uterus endometrial adenocarcinoma |
| G110E | Colon adenocarcinoma |
| G177D | Colon adenocarcinoma |
| G177S | Skin melanoma |
| G177V | Duodenum adenocarcinoma |
| G245V | Uterus endometrial adenocarcinoma papillary serous |
| G252S | Lung non-small cell lung carcinoma |
| G264W | Colon adenocarcinoma |
| H14Y | Lung adenocarcinoma |
| H151L | Colon adenocarcinoma |
| H172Y | Lung adenocarcinoma |
| H220Y | Skin melanoma |
| H220Y | Fallopian tube carcinosarcoma |
| H233Y | Unknown primary neuroendocrine tumor |
| H78R | Lung adenocarcinoma |
| I126S | Colon adenocarcinoma |
| I166L | Lung squamous cell carcinoma |
| I199V | Lung adenocarcinoma |
| I206M | Uterus endometrial adenocarcinoma |
| I243T | Colon adenocarcinoma |
| I258V | Unknown primary carcinoma |
| I38M | Breast carcinoma |
| I64T | Lung adenocarcinoma |
| 18K | Lung adenocarcinoma |
| K105Q | Lung adenocarcinoma |
| K105Q | Breast carcinoma |
| K105Q | Unknown primary undifferentiated small cell carcinoma |
| K162Q | Head and neck squamous cell carcinoma |
| K178E | Salivary gland adenoid cystic carcinoma |
| K25N | Uterus endometrial adenocarcinoma papillary serous |
| K280N | Uterus carcinosarcoma |
| K280N | Lung adenocarcinoma |
| K75T | Lung adenocarcinoma |
| L142F | Lung adenocarcinoma |
| L190V | Lung small cell undifferentiated carcinoma |
| L197P | Colon adenocarcinoma |
| L50V | Colon adenocarcinoma |
| L92H | Lung squamous cell carcinoma |
| M1I | Lung squamous cell carcinoma |
| M1I | Lung squamous cell carcinoma |
| M266I | Lung squamous cell carcinoma |
| M267I | Unknown primary melanoma |
| M59I | Colon adenocarcinoma |
| M59I | Uterus endometrial adenocarcinoma |
| N135S | Breast carcinoma |
| N135S | Lung adenosquamous carcinoma |
| N183S | Skin basal cell carcinoma |
| N183S | Skin melanoma |
| N200D | Prostate acinar adenocarcinoma |
| N96S | Uterus endometrial adenocarcinoma endometrioid |
| N96Y | Anus squamous cell carcinoma |
| P133S | Prostate acinar adenocarcinoma |
| P146S | Skin melanoma |
| P216H | Skin merkel cell carcinoma |
| P216S | Skin melanoma |
| P230S | Lung adenocarcinoma |
| P234R | Lung adenocarcinoma |
| P235L | Soft tissue angiosarcoma |
| P24A | Thyroid carcinoma |
| P24S | Soft tissue sarcoma |
| P43A | Breast invasive ductal carcinoma |
| P43L | Skin melanoma |
| P43Q | Bladder adenocarcinoma |
| Q100R | Uterus endometrial adenocarcinoma |
| Q107K | Prostate acinar adenocarcinoma |
| Q107K | Pancreas ductal adenocarcinoma |
| Q156H | Uterus endometrial adenocarcinoma |
| Q156K | Lung non-small cell lung carcinoma |
| Q173E | Uterus endometrial adenocarcinoma papillary serous |
| Q173H | Colon adenocarcinoma |
| Q275R | Lung non-small cell lung carcinoma |
| Q83H | Lung adenocarcinoma |
| Q83H | Fallopian tube serous carcinoma |
| Q83H | Lung small cell undifferentiated carcinoma |
| R113H | Colon adenocarcinoma |
| R113H | Colon adenocarcinoma (CRC) |
| R125Q | Colon adenocarcinoma (CRC) |
| R125Q | Skin squamous cell carcinoma (SCC) |
| R125Q | Rectum adenocarcinoma (CRC) |
| R125Q | Skin melanoma |
| R140I | Colon adenocarcinoma (CRC) |
| R140I | Colon adenocarcinoma (CRC) |
| R140T | Lung non-small cell lung carcinoma (NSCLC) (NOS) |
| R140T | Lung adenocarcinoma |
| R186T | Lung adenocarcinoma |
| R198K | Lung adenocarcinoma |
| R198T | Lung adenocarcinoma |
| R212K | Prostate acinar adenocarcinoma |
| R260C | Prostate acinar adenocarcinoma |
| R260H | Colon adenocarcinoma |
| R260H | Prostate acinar adenocarcinoma |
| R260H | Lung adenocarcinoma |
| R260H | Colon adenocarcinoma |
| R260H | Ampullary adenocarcinoma |
| R260H | Brain glioblastoma |
| R260H | Prostate acinar adenocarcinoma |
| R260H | Ovary epithelial carcinoma |
| R260H | Breast carcinoma |
| R260H | Uterus endometrial adenocarcinoma papillary serous |
| R260H | Rectum adenocarcinoma |
| R2M | Lung non-small cell lung carcinoma |
| R82I | Unknown primary carcinoma |
| R86W | Ovary carcinosarcoma |
| R86W | Colon adenocarcinoma |
| S195R | Unknown primary squamous cell carcinoma |
| T148A | Pancreas ductal adenocarcinoma |
| T180I | Colon adenocarcinoma |
| T181I | Lung adenocarcinoma |
| T182P | Lung adenocarcinoma |
| T196A | Lung squamous cell carcinoma |
| T201I | Lung squamous cell carcinoma |
| T290A | Gallbladder adenocarcinoma |
| T37A | Colon adenocarcinoma |
| T37K | Fallopian tube serous carcinoma |
| V130L | Ovary epithelial carcinoma |
| V143A | Lung adenocarcinoma |
| V147A | Pancreas ductal adenocarcinoma |
| V21A | Uterus endometrial adenocarcinoma |
| V21L | Lung adenocarcinoma |
| V253I | Pancreas ductal adenocarcinoma |
| V30A | Breast metaplastic carcinoma |
| V6I | Unknown primary sarcoma |
| V6I | Vagina squamous cell carcinoma |
| V76F | Lung adenocarcinoma |
| W13C | Lung large cell carcinoma |
| W57G | Pancreas ductal adenocarcinoma |
| Y112C | Duodenum adenocarcinoma |
| Y134C | Lung adenocarcinoma |
| Y160H | Kidney sarcomatoid carcinoma |
| Y160H | Pancreas ductal adenocarcinoma |
| Y28S | Skin melanoma |
| Y32C | Lung squamous cell carcinoma |
| Y81H | Prostate acinar adenocarcinoma |

Truncations

| | |
|---|---|
| *291Yext*42 | Colon adenocarcinoma |
| C272fs*13 | Uterus endometrial adenocarcinoma |
| C272fs*13 | Ovary serous carcinoma |
| D90fs*10 | Prostate acinar adenocarcinoma |
| E150* | Lung adenocarcinoma |
| E152* | Uterus endometrial adenocarcinoma endometrioid |
| E152* | Diffuse large b-cell lymphoma |
| E188fs*12 | Ovary epithelial carcinoma |
| E217* | Lung non-small cell lung carcinoma |
| F7fs*27 | Colon adenocarcinoma |
| K271fs*44 | Uterus endometrial adenocarcinoma |
| K271fs*44 | Skin adnexal carcinoma |
| K41* | Colon adenocarcinoma |
| P133fs*21 | Lung adenocarcinoma |
| Q107* | Ovary serous carcinoma |
| Q66fs*13 | Lung adenosquamous carcinoma |
| Q77* | Lung adenocarcinoma |
| R125* | Uterus endometrial adenocarcinoma endometrioid |
| R125* | Brain glioblastoma |
| R125* | Prostate undifferentiated carcinoma |
| R265fs*2 | Prostate acinar adenocarcinoma |
| S279* | Lung squamous cell carcinoma |
| Y134* | Pancreas ductal adenocarcinoma |

Splice Site Mutations

| | |
|---|---|
| splice site 53 − 49_82del79 | Lung adenocarcinoma |
| splice site 683 − 1G > A | Colon adenocarcinoma |
| splice site | Rectum adenocarcinoma |
| 790 + 1_790 + 4delGTAG | |
| splice site 790 + 1G > T | Lung adenocarcinoma |
| splice site 791 − 1G > A | Skin melanoma |
| splice site 791 − 1G > T | Colon adenocarcinoma |
| splice site 791 − 1G > T | Lung squamous cell carcinoma |

Insertion/Deletions

| | |
|---|---|
| E31_Y32insFTVTVPKDLYVVE | Thyroid papillary carcinoma |
| T203del | Breast invasive ductal carcinoma |

Missense Mutations with CD274 amplification

| | |
|---|---|
| E150G | Lung adenocarcinoma |
| V242I | Breast carcinoma |
| K25R | Unknown primary squamous cell carcinoma |

Multiple Missense Mutations

| | |
|---|---|
| I141M, F9L | Uterus endometrial adenocarcinoma endometrioid |
| K189M, E188K | Colon neuroendocrine carcinoma |

### Detection of CD274 Mutations

Certain aspects of the present disclosure relate to detection of one or more mutations in a CD274 gene of the present disclosure in a sample, e.g., a patient sample. In some embodiments, the one or more mutations in a CD274 gene are detected in vitro. Methods for detecting one or more mutations in a CD274 gene of the present disclosure are known in the art. For example, in some embodiments, one or more mutations in a CD274 gene are detected by sequencing part or all of the CD274 gene, e.g., by next-generation or other sequencing of DNA, RNA, or cDNA. In some embodiments, one or more mutations in a CD274 gene are detected by PCR amplification of DNA, RNA, or cDNA. In some embodiments, one or more mutations in a CD274 gene are detected by in situ hybridization using one or more polynucleotides that hybridize to the CD274 locus, or a rearrangement/fusion thereof, e.g., using fluorescence in situ hybridization (FISH). In some embodiments, one or more mutations in a CD274 gene are detected in a cancer cell, e.g., using tumor tissue, such as from a tumor biopsy or other tumor specimen. Exemplary and non-limiting methods for detecting one or more mutations in a CD274 gene in tumor samples are described herein.

Also provided herein are nucleic acid molecules, e.g., DNA (such as cDNA, genomic DNA or fragments thereof) or RNA (such as mRNA), that comprise or encode a CD274 gene or a portion thereof comprising one or more mutations described herein.

Also provided herein are PD-L1 polypeptides encoded by a CD274 gene, or a fragment thereof, comprising one or more mutations of the disclosure, and/or encoded by a nucleic acid molecule that comprises or encodes a CD274 gene or a portion thereof comprising one or more mutations described herein.

Also provided herein are methods of detecting a nucleic acid molecule that comprises or encodes a CD274 gene or a portion thereof comprising one or more mutations described herein, e.g., in a patient sample. In some embodiments, the nucleic acid molecule that comprises or encodes a CD274 gene or a portion thereof comprising one or more mutations described herein is detected in vitro. Methods for detecting nucleic acid molecules of the present disclosure are known in the art. For example, in some embodiments, a nucleic acid molecule that comprises or encodes a CD274 gene or a portion thereof comprising one or more mutations described herein is detected by sequencing part or all of the nucleic acid molecule, e.g., by next-generation or other sequencing of DNA, RNA, or cDNA. In some embodiments, a nucleic acid molecule that comprises or encodes a CD274 gene or a portion thereof comprising one or more mutations described herein is detected by PCR amplification of DNA, RNA, or cDNA. In some embodiments, a nucleic acid molecule that comprises or encodes a CD274 gene or a portion thereof comprising one or more mutations described herein is detected by in situ hybridization using one or more polynucleotides that hybridize to the nucleic acid molecule, e.g., using fluorescence in situ hybridization (FISH). In some embodiments, a nucleic acid molecule that comprises or encodes a CD274 gene or a portion thereof comprising one or more mutations described herein is detected in a cancer cell, e.g., using tumor tissue, such as from a tumor biopsy or other tumor specimen. Exemplary and non-limiting methods for detecting a nucleic acid molecule that comprises or encodes a CD274 gene or a portion thereof comprising one or more mutations of the disclosure are provided herein.

Also provided herein are methods of detecting a PD-L1 polypeptide encoded by a CD274 gene, or a fragment thereof, e.g., comprising one or more mutations of the disclosure. A PD-L1 polypeptide provided herein, or a fragment thereof, e.g., comprising one or more mutations of the disclosure, may be detected or measured, e.g., in a sample obtained from an individual, using any method known in the art, such as using antibodies (e.g., an antibody described herein), mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography).

In some embodiments, a PD-L1 polypeptide encoded by a CD274 gene, or a fragment thereof, e.g., comprising one or more mutations of the disclosure, can be distinguished from a reference polypeptide, e.g., a non-mutant or wild type PD-L1 protein or polypeptide, with an antibody or antibody fragment that reacts differentially with a mutant protein or polypeptide (e.g., a PD-L1 polypeptide encoded by a CD274 gene, or a fragment thereof, e.g., comprising one or more mutations of the disclosure) as compared to a reference protein or polypeptide. In some embodiments, a PD-L1 polypeptide encoded by a CD274 gene, or a fragment thereof, e.g., comprising one or more mutations of the disclosure, can be distinguished from a reference polypeptide, e.g., a non-mutant or wild type PD-L1 protein or polypeptide, by reaction with a detection reagent, e.g., a substrate, e.g., a substrate for catalytic activity.

In some aspects, methods of detection of a PD-L1 polypeptide encoded by a CD274 gene, or a fragment thereof, e.g., comprising one or more mutations of the disclosure, are provided, comprising contacting a sample, e.g., a sample described herein, comprising a PD-L1 polypeptide encoded by a CD274 gene, or a fragment thereof, e.g., comprising one or more mutations of the disclosure, with a detection reagent provided herein (e.g., an antibody of the disclosure), and determining if the PD-L1 polypeptide is present in the sample.

### Samples

In some embodiments, the sample is a formalin-fixed paraffin-embedded (FFPE) sample. In some embodiments, the sample comprises nucleic acids, e.g., genomic DNA, cDNA, or mRNA. In some embodiments, the sample is obtained from an individual having a cancer, such as a cancer described herein. A variety of materials (such as tissues) can be the source of the nucleic acid samples used in the methods provided herein. For example, the source of the sample can be solid tissue as from a fresh, frozen and/or preserved organ, tissue sample, biopsy, resection, smear, or aspirate; blood or any blood constituents; bodily fluids such as cerebrospinal fluid, amniotic fluid, urine, saliva, sputum, peritoneal fluid or interstitial fluid; or cells from any time in gestation or development of an individual. In some embodiments, the source of the sample is blood or blood constituents. In some embodiments, the source of the sample is a tumor sample. In some embodiments, the sample is or comprises biological tissue or fluid. In some embodiments, the sample can contain compounds that are not naturally intermixed with the tissue in nature, such as preservatives, anticoagulants, buffers, fixatives, nutrients, antibiotics or the like. In some embodiments, a nucleic acid molecule comprising one or more CD274 mutations of the disclosure is detected in a sample comprising genomic or subgenomic DNA fragments, or RNA, such as mRNA isolated from a sample, e.g., a tumor sample, a normal adjacent tissue (NAT) sample, a tissue sample, or a blood sample obtained from an individual. In some embodiments, the sample comprises cDNA derived from an mRNA sample or from a sample comprising mRNA. In some embodiments, the tissue is preserved as a frozen sample or as a formaldehyde- or paraformaldehyde-fixed paraffin-embedded (FFPE) tissue preparation. For example, the sample can be embedded in a matrix, e.g., an FFPE block or a frozen sample.

In some embodiments, the sample comprises cell-free DNA (cfDNA). In some embodiments, the sample comprises cell-free RNA (cfRNA). In some embodiments, the sample comprises circulating tumor DNA (ctDNA).

In some embodiments, a sample may be or comprise bone marrow; a bone marrow aspirate; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as ductal lavages or bronchoalveolar lavages; aspirates; scrapings; bone marrow specimens; tissue biopsy specimens; surgical specimens; other body fluids, secretions, and/or excretions; and/or cells therefrom. In some embodiments, a biological sample is or comprises cells obtained from an individual.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example nucleic acids or proteins extracted from a sample or obtained by subjecting a primary sample to techniques such as amplification or reverse transcription of mRNA, or isolation and/or purification of certain components.

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein. In some embodiments, the sample is acquired from a solid tumor, a soft tissue tumor or a metastatic lesion. In other embodiments, the sample includes tissue or cells from a surgical margin. In another embodiment, the sample includes one or more circulating tumor cells (CTCs) (e.g., a CTC acquired from a blood sample). In one embodiment, the sample is a cell not associated with a tumor, e.g., a non-tumor cell or a peripheral blood lymphocyte.

In some embodiments, the sample comprises tumor nucleic acids, such as nucleic acids from a tumor or a cancer sample, e.g., genomic DNA, RNA, or cDNA derived from RNA, from a tumor or cancer sample. In certain embodiments, a tumor nucleic acid sample is purified or isolated (e.g., it is removed from its natural state).

In some embodiments, the sample is a control nucleic acid sample or a reference nucleic acid sample, e.g., genomic DNA, RNA, or cDNA derived from RNA, not containing a mutation or gene fusion described herein. In certain embodiments, the reference or control nucleic acid sample comprises a wild type or a non-mutated sequence. In certain embodiments, the reference nucleic acid sample is purified or isolated (e.g., it is removed from its natural state). In other embodiments, the reference nucleic acid sample is from a non-tumor sample, e.g., a blood control, a normal adjacent tumor (NAT), or any other non-cancerous sample from the same or a different subject.

In some embodiments, a nucleic acid molecule comprising one or more mutations in a CD274 gene of the disclosure is detected in a sample comprising cell-free DNA (cfDNA), cell-free RNA, or circulating tumor DNA (ctDNA). In some embodiments, one or more mutations in a CD274 gene of the disclosure are detected in a sample comprising cell-free DNA (cfDNA), cell-free RNA, or circulating tumor DNA (ctDNA).

In some embodiments, a sample for use according to the methods of detection of a PD-L1 polypeptide encoded by a CD274 gene, or a fragment thereof, e.g., comprising one or more mutations of the disclosure, is a solid tissue, e.g., from a fresh, frozen and/or preserved organ, tissue sample, biopsy (e.g., a tumor biopsy), resection, smear, or aspirate; blood or any blood constituents; bodily fluids such as cerebrospinal fluid, amniotic fluid, urine, saliva, sputum, peritoneal fluid or interstitial fluid; or cells such as tumor cells. In some embodiments, the source of the sample is blood or blood constituents. In some embodiments, the source of the sample is a tumor sample. In some embodiments, the sample is or comprises biological tissue or fluid. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs).

In some embodiments, a sample for use according to the methods of detection of a PD-L1 polypeptide encoded by a CD274 gene, or a fragment thereof, e.g., comprising one or more mutations of the disclosure, is a sample of proteins isolated or obtained from a solid tissue, e.g., from a fresh, frozen and/or preserved organ, tissue sample, biopsy (e.g., a tumor biopsy), resection, smear, or aspirate; from blood or any blood constituents; from bodily fluids such as cerebrospinal fluid, amniotic fluid, urine, saliva, sputum, peritoneal fluid or interstitial fluid; or from cells such as tumor cells. In some embodiments, the sample is a sample of proteins isolated or obtained from a preserved sample, such as a frozen sample or a formaldehyde- or paraformaldehyde-fixed paraffin-embedded (FFPE) tissue preparation. In some embodiments, the sample is a sample of proteins isolated or obtained from circulating tumor cells (CTCs). In some embodiments, the sample can contain compounds that are not naturally intermixed with the tissue in nature, such as preservatives, anticoagulants, buffers, fixatives, nutrients, antibiotics or the like.

In some embodiments, a sample may be or comprise bone marrow; a bone marrow aspirate; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as ductal lavages or bronchoalveolar lavages; aspirates; scrapings; bone marrow specimens; tissue biopsy specimens; surgical specimens; other body fluids, secretions, and/or excretions; and/or cells therefrom. In some embodiments, a biological sample is or comprises cells obtained from an individual.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example, proteins extracted from a sample or obtained by subjecting a primary sample to techniques such as isolation and/or purification of certain components.

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein. In some embodiments, the sample is acquired from a solid tumor, a soft tissue tumor or a metastatic lesion. In other embodiments, the sample includes tissue or cells from a surgical margin. In another embodiment, the sample includes one or more circulating tumor cells (CTCs) (e.g., a CTC acquired from a blood sample). In one embodiment, the sample is a cell not associated with a tumor, e.g., a non-tumor cell or a peripheral blood lymphocyte.

In some embodiments, the sample comprises tumor proteins or polypeptides, such as proteins or polypeptides from a tumor or a cancer sample. In certain embodiments, the proteins are purified or isolated (e.g., removed from their natural state).

In some embodiments, the sample is a control sample or a reference sample, e.g., not containing a PD-L1 polypeptide encoded by a CD274 gene, or a fragment thereof, comprising one or more mutations of the disclosure. In certain embodiments, the reference sample is purified or isolated (e.g., it is removed from its natural state). In other embodiments, the reference sample is from a non-tumor sample, e.g., a blood control, a normal adjacent tumor (NAT), or any other non-cancerous sample from the same or a different subject.

### Detection Methods

In some embodiments, one or more mutations in a CD274 gene of the disclosure, e.g., in a nucleic acid molecule comprising or encoding a CD274 gene or a portion thereof, may be detected using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect nucleic acid molecules, are described in U.S. Pat. No. 9,340,830 and in WO2012092426A1, which are hereby incorporated by reference in their entirety.

In some embodiments, one or more mutations in a CD274 gene of the disclosure, e.g., in a nucleic acid molecule comprising or encoding a CD274 gene or a portion thereof, are detected using an in situ hybridization method, such as a fluorescence in situ hybridization (FISH) method.

In some embodiments, FISH analysis is used to identify a chromosomal rearrangement resulting in the CD274 mutations as described herein. In some embodiments, FISH analysis is used to identify an RNA molecule comprising one or more mutations in a CD274 gene described herein. Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH.

In some embodiments, one or more mutations in a CD274 gene of the disclosure, e.g., in a nucleic acid molecule comprising or encoding a CD274 gene or a portion thereof, are detected using an array-based method, such as array-based comparative genomic hybridization (CGH) methods. In array-based CGH methods, a first sample of nucleic acids (e.g., from a sample, such as from a tumor) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor) is labeled and hybridized to a second array (with identical content) and absolute signals are read. Copy number differences are calculated based on absolute signals from the two arrays.

In some embodiments, one or more mutations in a CD274 gene of the disclosure, e.g., in a nucleic acid molecule comprising or encoding a CD274 gene or a portion thereof, are detected using an amplification-based method. As is known in the art, in such amplification-based methods, a sample of nucleic acids, such as a sample obtained from an individual or from a tumor, is used as a template in an amplification reaction (e.g., Polymerase Chain Reaction (PCR)) using one or more oligonucleotides or primers, e.g., such as one or more oligonucleotides or primers provided herein. The presence of one or more mutations in a CD274 gene of the disclosure, e.g., in a nucleic acid molecule comprising or encoding a CD274 gene or a portion thereof, in the sample can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

In some embodiments, one or more mutations in a CD274 gene of the disclosure, e.g., in a nucleic acid molecule comprising or encoding a CD274 gene or a portion thereof, are detected using a sequencing method. Any method of sequencing known in the art can be used to detect one or more mutations in a CD274 gene of the disclosure, e.g., in a nucleic acid molecule comprising or encoding a CD274 gene or a portion thereof. Exemplary sequencing methods that may be used include those based on techniques developed by Maxam and Gilbert or Sanger. Automated sequencing procedures may also be used, e.g., including sequencing by mass spectrometry.

In some embodiments, one or more mutations in a CD274 gene of the disclosure, e.g., in a nucleic acid molecule comprising or encoding a CD274 gene or a portion thereof, are detected using hybrid capture-based sequencing (hybrid capture-based NGS), e.g., using adaptor ligation-based libraries. See, e.g., Frampton, G. M. et al. (2013) Nat. Biotech. 31:1023-1031. In some embodiments, one or more mutations in a CD274 gene of the disclosure, e.g., in a nucleic acid molecule comprising or encoding a CD274 gene or a portion thereof, are detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 105 molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used according to the methods provided herein, include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLiD sequencer from Applied Biosystems (Foster City, CA, USA). Additional exemplary methods and platforms that may be used according to the methods provided herein include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

### Detection Reagents

In some aspects, provided herein are reagents for detecting one or more mutations in a CD274 gene of the disclosure, e.g., in a nucleic acid molecule comprising or encoding a CD274 gene or a portion thereof, e.g., according to the methods of detection provided herein. In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid, e.g., a nucleic acid that comprises or encodes a CD274 gene or a portion thereof comprising one or more mutations described herein.

In some embodiments, nucleic acids corresponding to the CD274 gene are captured (e.g., from amplified nucleic acids) by hybridization with a bait molecule. Provided herein are baits suitable for the detection of a nucleic acid molecule of the disclosure, e.g., a nucleic acid molecule that comprises or encodes a CD274 gene or a portion thereof comprising one or more mutations described herein.

In some embodiments, the bait comprises a capture nucleic acid molecule configured to hybridize to a target nucleic acid molecule that comprises or encodes a CD274 gene or a portion thereof comprising one or more mutations described herein, or a fragment or portion thereof. In some embodiments, the capture nucleic acid molecule is configured to hybridize to CD274 nucleotide sequences on the target nucleic acid molecule. In some embodiments, the capture nucleic acid molecule is configured to hybridize to a fragment of the target nucleic acid molecule. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule is between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length.

In some embodiments, the capture nucleic acid molecule is configured to hybridize to a breakpoint in a nucleic acid molecule that comprises or encodes a CD274 gene or a portion thereof comprising one or more mutations described herein, e.g., in a breakpoint resulting from a chromosomal rearrangement or gene fusion, and may further hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the capture nucleic acid molecule is configured to hybridize to a nucleotide sequence in an intron or an exon of CD274, or in a breakpoint in a nucleic acid molecule that comprises or encodes a CD274 gene or a portion thereof comprising one or more mutations described herein (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the bait comprises a nucleotide sequence configured to hybridize to a nucleotide sequence in an intron or an exon of a CD274 gene comprising one or more mutations described herein, or in a breakpoint in a nucleic acid molecule that comprises or encodes a CD274 gene or a portion thereof comprising one or more mutations described herein (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides), wherein the breakpoint joints an intron or an exon of the CD274 gene to an intron or an exon of another gene. In some embodiments, the bait comprises a nucleotide sequence configured to hybridize to a breakpoint in a nucleic acid molecule that comprises or encodes a CD274 gene or a portion thereof (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides), wherein the breakpoint joints an intron or an exon of the CD274 gene to an intron or an exon of another gene.

In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises about 150 nucleotides. In some embodiments, the capture nucleic acid molecule is about 150 nucleotides. In some embodiments, the capture nucleic acid molecule comprises about 170 nucleotides. In some embodiments, the capture nucleic acid molecule is about 170 nucleotides.

In some embodiments, a bait provided herein comprises a DNA, RNA, or a DNA/RNA molecule. In some embodiments, a bait provided herein includes a label or a tag. In some embodiments, the label or tag is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag, e.g., that allows capture and isolation of a hybrid formed by a bait and a nucleic acid hybridized to the bait. In some embodiments, the affinity tag is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1 and/or or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides. In some embodiments, a bait provided herein is between about 130 nucleotides and about 230 nucleotides. In some embodiments, a bait provided herein is between about 150 nucleotides and about 200 nucleotides. In some embodiments, a bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the bait) are universal arbitrary tails, e.g., suitable for PCR amplification.

The baits described herein can be used for selection of exons and short target sequences.

In some embodiments, a bait of the disclosure distinguishes a nucleic acid, e.g., a genomic or transcribed nucleic acid, e.g., a cDNA or RNA, comprising or encoding a CD274 gene or a portion thereof comprising one or more mutations described herein, from a reference nucleotide sequence, e.g., a nucleotide sequence not comprising one or more mutations described herein.

In some embodiments, the bait hybridizes to a CD274 mutation described herein, or to a CD274 breakpoint described herein (e.g., that results or is associated with one or more CD274 mutations described herein), and a sequence on either side of the mutation or breakpoint (e.g., any of 1, 2, 3, 4, 5, 6, 7, 8, 9, or 10 nucleotides on either side of the mutation or breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the mutation or breakpoint).

In some embodiments, a bait of the disclosure specifically hybridizes to a mutation (e.g., one or more CD274 mutations described herein, or to a breakpoint, rearrangement, inversion, duplication, deletion, insertion or translocation resulting in or associated with one or more CD274 mutations described herein)

In some embodiments, a bait of the disclosure is suitable for solution phase hybridization.

Also provided herein are probes, e.g., nucleic acid molecules, suitable for the detection of a nucleic acid molecule that comprises or encodes a CD274 gene or a portion thereof comprising one or more mutations described herein. In some embodiments, a probe provided herein comprises a nucleic acid sequence configured to hybridize to a target nucleic acid molecule that comprises or encodes a CD274 gene or a portion thereof comprising one or more mutations described herein. In some embodiments, the probe is configured to hybridize to CD274 nucleotide sequences on the target nucleic acid molecule, or to a fragment or portion thereof. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides.

In some embodiments, the probe comprises a nucleotide sequence configured to hybridize to a breakpoint in a nucleic acid molecule that comprises or encodes a CD274 gene or a portion thereof comprising one or more mutations described herein, e.g., in a breakpoint resulting from a chromosomal rearrangement or gene fusion, and may be further configured to hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the probe comprises a nucleotide sequence configured to hybridize to a nucleotide sequence in an intron or an exon of a CD274 gene comprising one or more mutations described herein, or in a breakpoint in a nucleic acid molecule that comprises or encodes a CD274 gene or a portion thereof comprising one or more mutations described herein (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to a nucleotide sequence in an intron or an exon of a CD274 gene comprising one or more mutations described herein, or in a breakpoint in a nucleic acid molecule that comprises or encodes a CD274 gene or a portion thereof comprising one or more mutations described herein (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides), wherein the breakpoint joints an intron or an exon of the CD274 gene to an intron or an exon of another gene. In some embodiments, the probe comprises a nucleotide sequence configured to hybridize to a breakpoint in a nucleic acid molecule that comprises or encodes a CD274 gene or a portion thereof (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides), wherein the breakpoint joints an intron or an exon of the CD274 gene to an intron or an exon of another gene.

In some embodiments, the probe comprises a nucleic acid molecule which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides.

In some embodiments, a probe provided herein comprises a DNA, RNA, or a DNA/RNA molecule. In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid hybridized to the probe. In some embodiments, the affinity tag is an antibody, an antibody fragment, biotin, or any other suitable affinity tag or reagent known in the art. In some embodiments, a probe is suitable for solution phase hybridization.

In some embodiments, probes provided herein may be used according to the methods of detection of one or more CD274 mutations provided herein, e.g., for the detection of a nucleic acid molecule that comprises or encodes a CD274 gene or a portion thereof comprising one or more mutations described herein. For example, a probe provided herein may be used for detecting one or more CD274 mutations provided herein, e.g., in a nucleic acid molecule that comprises or encodes a CD274 gene or a portion thereof comprising one or more mutations described herein, in a sample, e.g., a sample obtained from an individual. In some embodiments, the probe may be used for identifying cells or tissues that express a CD274 gene comprising one or more mutations provided herein, or a nucleic acid molecule that comprises or encodes a CD274 gene or a portion thereof comprising one or more mutations described herein, e.g., by measuring levels of the CD274 gene comprising one or more mutations provided herein, or the nucleic acid molecule that comprises or encodes a CD274 gene or a portion thereof comprising one or more mutations described herein. In some embodiments, the probe may be used for detecting levels of a nucleic acid molecule that comprises or encodes a CD274 gene or a portion thereof comprising one or more mutations described herein, e.g., mRNA levels, in a sample of cells from an individual.

In some embodiments, a probe provided herein specifically hybridizes to a nucleic acid comprising a rearrangement (e.g., a deletion, inversion, insertion, duplication, or other rearrangement) resulting in or associated with one or more CD274 mutations described herein.

In some embodiments, a probe of the disclosure distinguishes a nucleic acid, e.g., a genomic or transcribed nucleic acid, e.g., a cDNA or RNA, having one or more CD274 mutations described herein, from a reference nucleotide sequence, e.g., a nucleotide sequence not having the one or more mutations.

Also provided herein are isolated pairs of allele-specific probes, wherein, for example, the first probe of the pair specifically hybridizes to a nucleic acid molecule that comprises or encodes a CD274 gene or a portion thereof comprising one or more mutations described herein, and the second probe of the pair specifically hybridizes to a corresponding wild type sequence (e.g., a wild type CD274 nucleic acid molecule). Probe pairs can be designed and produced for any of the nucleic acid molecules described herein (e.g., comprising or encoding a CD274 gene or a portion thereof comprising one or more mutations described herein) and are useful in detecting a somatic mutation in a sample. In some embodiments, a first probe of a pair specifically hybridizes to a mutation (e.g., one or more CD274 mutations described herein, or to a breakpoint, rearrangement, inversion, duplication, deletion, insertion or translocation resulting in or associated with one or more CD274 mutations described herein), and a second probe of a pair specifically hybridizes to a sequence upstream or downstream of the mutation.

In some embodiments, one or more probes provided herein are suitable for use in in situ hybridization methods, e.g., as described above, such as FISH.

Chromosomal probes, e.g., for use in the FISH methods described herein, are typically about 50 to about 10⁵ nucleotides in length. Longer probes typically comprise smaller fragments of about 100 to about 500 nucleotides. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA, such as an RNA that comprises or encodes a CD274 gene or a portion thereof comprising one or more mutations described herein.

In some embodiments, probes, such as probes for use in the FISH methods described herein, are used for determining whether a cytogenetic abnormality is present in one or more cells, e.g., in a region of a chromosome or an RNA bound by one or more probes provided herein. The cytogenetic abnormality may be a cytogenetic abnormality that results in or is associated with one or more CD274 mutations described herein. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations), intra-chromosomal inversions, point mutations, deletions, gene copy number changes, germ-line mutations, and gene expression level changes.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)- or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin (BIO)-11-dUTP, Digoxygenin (DIG)-11-dUTP and Dinitrophenyl (DNP)-11-dUTP. Probes can also be indirectly labeled with biotin or digoxygenin, or labeled with radioactive isotopes such as ³²P and 3H, and secondary detection molecules are used, or further processing is performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

In some embodiments, the probe hybridizes to a CD274 mutation described herein, or to a CD274 breakpoint described herein (e.g., that results or is associated with one or more CD274 mutations described herein), and a sequence on either side of the mutation or breakpoint (e.g., any of 1, 2, 3, 4, 5, 6, 7, 8, 9, or 10 nucleotides on either side of the mutation or breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the mutation or breakpoint).

In some aspects, provided herein are oligonucleotides, e.g., useful as primers. In some embodiments, an oligonucleotide, e.g., a primer, provided herein comprises a nucleotide sequence configured to hybridize to a target nucleic acid molecule that comprises or encodes a CD274 gene or a portion thereof comprising one or more mutations described herein, or a fragment or portion thereof. In some embodiments, the oligonucleotide comprises a nucleotide sequence configured to hybridize to CD274 nucleotide sequences on the target nucleic acid molecule. In some embodiments, the oligonucleotide comprises a nucleotide sequence configured to hybridize to a fragment or portion of the target nucleic acid molecule.

In some embodiments, the oligonucleotide, e.g., the primer, comprises a nucleotide sequence configured to hybridize to a breakpoint in a nucleic acid molecule that comprises or encodes a CD274 gene or a portion thereof comprising one or more mutations described herein, and may be further configured to hybridize to between about 10 and about 12, about 12 and about 15, about 15 and about 17, about 17 and about 20, about 20 and about 25, or about 25 and about 30, or more nucleotides flanking either side of the breakpoint.

In some embodiments, the oligonucleotide, e.g., the primer, comprises a nucleotide sequence configured to hybridize to a nucleotide sequence in an intron or an exon of CD274, or in a breakpoint in a nucleic acid molecule that comprises or encodes a CD274 gene or a portion thereof comprising one or more mutations described herein (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to a nucleotide sequence in an intron or an exon of a CD274 gene comprising one or more mutations described herein, or in a breakpoint in a nucleic acid molecule that comprises or encodes a CD274 gene or a portion thereof comprising one or more mutations described herein (e.g., plus or minus any of between about 10 and about 12, about 12 and about 15, about 15 and about 17, about 17 and about 20, about 20 and about 25, or about 25 and about 30, or more nucleotides), wherein the breakpoint joints an intron or an exon of the CD274 gene to an intron or an exon of another gene. In some embodiments, the oligonucleotide, e.g., the primer, comprises a nucleotide sequence configured to hybridize to a breakpoint in a nucleic acid molecule that comprises or encodes a CD274 gene or a portion thereof (e.g., plus or minus any of between about 10 and about 12, about 12 and about 15, about 15 and about 17, about 17 and about 20, about 20 and about 25, or about 25 and about 30, or more nucleotides), wherein the breakpoint joints an intron or an exon of the CD274 gene to an intron or an exon of another gene.

In some embodiments, the oligonucleotide comprises a nucleotide sequence corresponding to a nucleic acid molecule that comprises or encodes a CD274 gene or a portion thereof comprising one or more mutations described herein. In some embodiments, the oligonucleotide comprises a nucleotide sequence corresponding to a fragment or a portion of a nucleic acid molecule that comprises or encodes a CD274 gene or a portion thereof comprising one or more mutations described herein. In some embodiments, the fragment or portion comprises between about 10 and about 30 nucleotides, between about 12 and about 20 nucleotides, or between about 12 and about 17 nucleotides. In some embodiments, the oligonucleotide comprises a nucleotide sequence complementary to a nucleic acid molecule that comprises or encodes a CD274 gene or a portion thereof comprising one or more mutations described herein. In some embodiments, the oligonucleotide comprises a nucleotide sequence complementary to a nucleic acid molecule that comprises or encodes a CD274 gene or a portion thereof comprising one or more mutations described herein. In some embodiments, the fragment or portion comprises between about 10 and about 30 nucleotides, between about 12 and about 20 nucleotides, or between about 12 and about 17 nucleotides.

In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method. In some embodiments, the oligonucleotide may be used to amplify a nucleic acid molecule that comprises or encodes a CD274 gene or a portion thereof comprising one or more mutations described herein, or a fragment thereof, e.g., using PCR. In some embodiments, the oligonucleotide may be used to sequence a nucleic acid molecule that comprises or encodes a CD274 gene or a portion thereof comprising one or more mutations described herein. In some embodiments, the oligonucleotide may be used to amplify a nucleic acid molecule that comprises a breakpoint resulting in or associated with one or more CD274 mutations described herein, e.g., using PCR. In some embodiments, the oligonucleotide may be used to sequence a nucleic acid molecule that comprises a breakpoint resulting in or associated with one or more CD274 mutations described herein, e.g., using PCR.

In some embodiments, pairs of oligonucleotides, e.g., pairs of primers, are provided herein, which are configured to hybridize to a nucleic acid molecule that comprises or encodes a CD274 gene or a portion thereof comprising one or more mutations described herein. In some embodiments, a pair of oligonucleotides of the disclosure may be used for directing amplification of the nucleic acid molecule that comprises or encodes a CD274 gene or a portion thereof comprising one or more mutations described herein, e.g., using a PCR reaction. In some embodiments, pairs of oligonucleotides, e.g., pairs of primers, are provided herein, which are configured to hybridize to a nucleic acid molecule that comprises a breakpoint resulting in or associated with one or more CD274 mutations described herein, e.g., for use in directing amplification of the nucleic acid molecule or fragment thereof, e.g., using a PCR reaction.

In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, comprising or encoding a CD274 gene or a portion thereof comprising one or more mutations described herein, from a reference nucleotide sequence, e.g., a nucleotide sequence not having the one or more mutations. In some embodiments, an oligonucleotide, e.g., a primer, of the disclosure distinguishes a nucleic acid, e.g., a genomic or transcribed nucleic acid, e.g., a cDNA or RNA, comprising a breakpoint resulting in or associated with one or more C274 mutations described herein, from a reference nucleotide sequence, e.g., a nucleotide sequence not having the breakpoint.

In one aspect, provided herein is a primer or primer set for amplifying a nucleic acid molecule comprising a cytogenetic abnormality such as an alteration, rearrangement, chromosomal inversion, deletion, translocation, duplication, or other rearrangement resulting in or associated with one or more CD274 mutations described herein. In another aspect, provided herein is a primer or primer set for amplifying a nucleic acid molecule comprising an alteration, rearrangement, chromosomal inversion, insertion, deletion, translocation, duplication or other rearrangement resulting in or associated with one or more CD274 mutations described herein. In certain aspects, provided herein are allele-specific oligonucleotides, e.g., primers, wherein a first oligonucleotide of a pair specifically hybridizes to a mutation (e.g., one or more CD274 mutations described herein), and a second oligonucleotide of a pair specifically hybridizes to a sequence upstream or downstream of the mutation. In certain aspects, provided herein are pairs of oligonucleotides, e.g., primers, wherein a first oligonucleotide of a pair specifically hybridizes to a sequence upstream of a mutation (e.g., one or more CD274 mutations described herein), and a second oligonucleotide of the pair specifically hybridizes to a sequence downstream of the mutation.

In some embodiments, the oligonucleotide, e.g., the primer, hybridizes to a CD274 mutation described herein, or to a CD274 breakpoint described herein (e.g., that results or is associated with one or more CD274 mutations described herein), and a sequence on either side of the mutation or breakpoint (e.g., any of 1, 2, 3, 4, 5, 6, 7, 8, 9, or 10 nucleotides on either side of the mutation or breakpoint).

### Tumor Mutational Burden

In some embodiments, the methods provided herein comprise acquiring knowledge of or detecting the level of tumor mutational burden in a cancer of the disclosure. In some embodiments, acquiring knowledge of or detecting the level of tumor mutational burden in a cancer of the disclosure comprises measuring the level of tumor mutational burden in a sample, e.g., in a sample from a cancer or a tumor, obtained from an individual.

In some embodiments, tumor mutational burden is assessed in sample from an individual, such as sample described herein. In some embodiments, the sample from the individual comprises fluid, cells, or tissue. In some embodiments, the sample from the individual comprises a tumor biopsy or a circulating tumor cell. In some embodiments, the sample from the individual comprises nucleic acids. In some embodiments, the sample from the individual comprises mRNA, genomic DNA, circulating tumor DNA, cell-free DNA, or cell-free RNA.

In some embodiments, tumor mutational burden is measured using any suitable method known in the art. For example, tumor mutational burden may be measured using whole-exome sequencing (WES), next-generation sequencing, whole genome sequencing, gene-targeted sequencing, or sequencing of a panel of genes, e.g., panels including cancer-related genes. See, e.g., Melendez et al., Transl Lung Cancer Res (2018) 7(6):661-667. In some embodiments, tumor mutational burden is measured using gene-targeted sequencing, e.g., using a nucleic acid hybridization-capture method, e.g., coupled with sequencing. See, e.g., Fancello et al., J Immunother Cancer (2019) 7:183.

In some embodiments, tumor mutational burden is measured according to the methods provided in WO2017151524A1, which is hereby incorporated by reference in its entirety.

In some embodiments, tumor mutational burden is measured in the sample by whole exome sequencing. In some embodiments, tumor mutational burden is measured in the sample using next-generation sequencing. In some embodiments, tumor mutational burden is measured in the sample using whole genome sequencing. In some embodiments, tumor mutational burden is measured in the sample by gene-targeted sequencing. In some embodiments, tumor mutational burden is measured on between about 0.8 Mb and about 1.3 Mb of sequenced DNA. In some embodiments, tumor mutational burden is measured on any of about 0.8 Mb, about 0.81 Mb, about 0.82 Mb, about 0.83 Mb, about 0.84 Mb, about 0.85 Mb, about 0.86 Mb, about 0.87 Mb, about 0.88 Mb, about 0.89 Mb, about 0.9 Mb, about 0.91 Mb, about 0.92 Mb, about 0.93 Mb, about 0.94 Mb, about 0.95 Mb, about 0.96 Mb, about 0.97 Mb, about 0.98 Mb, about 0.99 Mb, about 1 Mb, about 1.01 Mb, about 1.02 Mb, about 1.03 Mb, about 1.04 Mb, about 1.05 Mb, about 1.06 Mb, about 1.07 Mb, about 1.08 Mb, about 1.09 Mb, about 1.1 Mb, about 1.2 Mb, or about 1.3 Mb of sequenced DNA. In some embodiments, tumor mutational burden is measured on about 0.8 Mb of sequenced DNA. In some embodiments, tumor mutational burden is measured on between about 0.83 Mb and about 1.14 Mb of sequenced DNA. In some embodiments, tumor mutational burden is measured on up to about 1.24 Mb of sequenced DNA. In some embodiments, tumor mutational burden is measured on up to about 1.1 Mb of sequenced DNA. In some embodiments, tumor mutational burden is measured on about 0.79 Mb of sequenced DNA.

In some embodiments, a cancer of the disclosure has a tumor mutational burden of less than about 10 mut/Mb, e.g., any of about 9.9 mut/Mb, about 9.8 mut/Mb, about 9.6 mut/Mb, about 9.4 mut/Mb, about 9.2 mut/Mb, about 9 mut/Mb, about 8.8 mut/Mb, about 8.6 mut/Mb, about 8.4 mut/Mb, about 8.2 mut/Mb, about 8 mut/Mb, about 7.8 mut/Mb, about 7.6 mut/Mb, about 7.4 mut/Mb, about 7.2 mut/Mb, about 7 mut/Mb, about 6.8 mut/Mb, about 6.6 mut/Mb, about 6.4 mut/Mb, about 6.2 mut/Mb, about 6 mut/Mb, about 5.8 mut/Mb, about 5.6 mut/Mb, about 5.4 mut/Mb, about 5.2 mut/Mb, about 5 mut/Mb, about 4.8 mut/Mb, about 4.6 mut/Mb, about 4.4 mut/Mb, about 4.2 mut/Mb, about 4 mut/Mb, about 3.8 mut/Mb, about 3.6 mut/Mb, about 3.4 mut/Mb, about 3.2 mut/Mb, about 3 mut/Mb, about 2.8 mut/Mb, about 2.6 mut/Mb, about 2.4 mut/Mb, about 2.2 mut/Mb, about 2 mut/Mb, about 1.8 mut/Mb, about 1.6 mut/Mb, about 1.4 mut/Mb, about 1.2 mut/Mb, about 1 mut/Mb, about 0.8 mut/Mb, about 0.6 mut/Mb, about 0.4 mut/Mb, about 0.2 mut/Mb, or less.

In some embodiments, a cancer of the disclosure has a high tumor mutational burden, e.g., of at least about 10 mut/Mb. In some embodiments, the cancer has a tumor mutational burden of at least about 10 mut/Mb. In some embodiments, the cancer has a tumor mutational burden of at least about 20 mut/Mb. In some embodiments, the cancer has a tumor mutational burden of any of between about 10 mut/Mb and about 15 mut/Mb, between about 15 mut/Mb and about 20 mut/Mb, between about 20 mut/Mb and about 25 mut/Mb, between about 25 mut/Mb and about 30 mut/Mb, between about 30 mut/Mb and about 35 mut/Mb, between about 35 mut/Mb and about 40 mut/Mb, between about 40 mut/Mb and about 45 mut/Mb, between about 45 mut/Mb and about 50 mut/Mb, between about 50 mut/Mb and about 55 mut/Mb, between about 55 mut/Mb and about 60 mut/Mb, between about 60 mut/Mb and about 65 mut/Mb, between about 65 mut/Mb and about 70 mut/Mb, between about 70 mut/Mb and about 75 mut/Mb, between about 75 mut/Mb and about 80 mut/Mb, between about 80 mut/Mb and about 85 mut/Mb, between about 85 mut/Mb and about 90 mut/Mb, between about 90 mut/Mb and about 95 mut/Mb, or between about 95 mut/Mb and about 100 mut/Mb. In some embodiments, the cancer has a tumor mutational burden of any of between about 100 mut/Mb and about 110 mut/Mb, between about 110 mut/Mb and about 120 mut/Mb, between about 120 mut/Mb and about 130 mut/Mb, between about 130 mut/Mb and about 140 mut/Mb, between about 140 mut/Mb and about 150 mut/Mb, between about 150 mut/Mb and about 160 mut/Mb, between about 160 mut/Mb and about 170 mut/Mb, between about 170 mut/Mb and about 180 mut/Mb, between about 180 mut/Mb and about 190 mut/Mb, between about 190 mut/Mb and about 200 mut/Mb, between about 210 mut/Mb and about 220 mut/Mb, between about 220 mut/Mb and about 230 mut/Mb, between about 230 mut/Mb and about 240 mut/Mb, between about 240 mut/Mb and about 250 mut/Mb, between about 250 mut/Mb and about 260 mut/Mb, between about 260 mut/Mb and about 270 mut/Mb, between about 270 mut/Mb and about 280 mut/Mb, between about 280 mut/Mb and about 290 mut/Mb, between about 290 mut/Mb and about 300 mut/Mb, between about 300 mut/Mb and about 310 mut/Mb, between about 310 mut/Mb and about 320 mut/Mb, between about 320 mut/Mb and about 330 mut/Mb, between about 330 mut/Mb and about 340 mut/Mb, between about 340 mut/Mb and about 350 mut/Mb, between about 350 mut/Mb and about 360 mut/Mb, between about 360 mut/Mb and about 370 mut/Mb, between about 370 mut/Mb and about 380 mut/Mb, between about 380 mut/Mb and about 390 mut/Mb, between about 390 mut/Mb and about 400 mut/Mb, or more than 400 mut/Mb. In some embodiments, the cancer has a TMB of at least about 100 mut/Mb, at least about 110 mut/Mb, at least about 120 mut/Mb, at least about 130 mut/Mb, at least about 140 mut/Mb, at least about 150 mut/Mb, or more.

In some embodiments, measuring tumor mutational burden comprises assessing mutations in a sample derived from a cancer in an individual. In some embodiments, measuring tumor mutational burden comprises assessing mutations in a sample derived from a cancer in an individual and in a matched normal sample, e.g., a sample from the individual derived from a tissue or other source that is free of the cancer.

### PD-L1 Expression

In some embodiments, the methods provided herein comprise acquiring knowledge of or detecting the level of PD-L1 expression in a cancer of the disclosure. In some embodiments, acquiring knowledge of or detecting the level of PD-L1 expression in a cancer of the disclosure comprises measuring PD-L1 expression in a sample, e.g., in a sample from a cancer or a tumor, obtained from an individual.

Any suitable method for measuring PD-L1 expression in a sample from an individual may be used. For example, the level of PD-L1 expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), MassARRAY, proteomics (e.g., mass spectrometry), quantitative blood based assays (as for example serum ELISA), biochemical enzymatic activity assays, in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”). Multiplexed immunoassays such as those available from Rules Based Medicine or Meso Scale Discovery (“MSD”) may also be used.

In some embodiments, PD-L1 expression in a sample from an individual is measured based on the level of PD-L1 mRNA in the sample. Any suitable method for measuring mRNA expression in a sample from an individual may be used. For example, the level of PD-L1 mRNA expression may be measured using in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”).

In some embodiments, PD-L1 expression in a sample from an individual is measured based on the level of PD-L1 protein in the sample. Any suitable method for measuring protein expression in a sample from an individual may be used. For example, the level of PD-L1 protein expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), proteomics (e.g., mass spectrometry), quantitative blood based assays (as for example serum ELISA), biochemical enzymatic activity assays, or multiplexed immunoassays such as those available from Rules Based Medicine or Meso Scale Discovery (“MSD”).

In some embodiments, a cancer provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs) express PD-L1 protein and/or PD-L1 mRNA (e.g., are positive for PD-L1 protein and/or PD-L1 mRNA). In some embodiments, a cancer provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) express PD-L1 protein and/or PD-L1 mRNA (e.g., are positive for PD-L1 protein and/or PD-L1 mRNA). In some embodiments, a cancer provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor cells express PD-L1 protein and/or PD-L1 mRNA (e.g., are positive for PD-L1 protein and/or PD-L1 mRNA).

In some embodiments, a cancer provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs) in a sample from an individual express PD-L1 protein and/or PD-L1 mRNA (e.g., are positive for PD-L1 protein and/or PD-L1 mRNA). In some embodiments, a cancer provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) in a sample from an individual express PD-L1 protein and/or PD-L1 mRNA (e.g., are positive for PD-L1 protein and/or PD-L1 mRNA). In some embodiments, a cancer provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor cells in a sample from an individual express PD-L1 protein and/or PD-L1 mRNA (e.g., are positive for PD-L1 protein and/or PD-L1 mRNA).

In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs) in the sample express PD-L1 protein and/or PD-L1 mRNA (e.g., are positive for PD-L1 protein and/or PD-L1 mRNA). In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) in the sample express PD-L1 protein and/or PD-L1 mRNA (e.g., are positive for PD-L1 protein and/or PD-L1 mRNA). In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor cells in the sample express PD-L1 protein and/or PD-L1 mRNA (e.g., are positive for PD-L1 protein and/or PD-L1 mRNA).

In some embodiments, the level of PD-L1 protein and/or PD-L1 mRNA is assessed in sample from an individual, such as a sample described herein. In some embodiments, the sample from the individual comprises fluid, cells, or tissue. In some embodiments, the sample from the individual comprises a tumor biopsy or a circulating tumor cell. In some embodiments, the sample is obtained or derived from a cancer, e.g., a cancer of the disclosure.

In some embodiments of any of the methods provided herein, a sample from an individual, e.g., an individual having a cancer of the disclosure, is determined to be PD-L1-negative if 0% of tumor cells in the sample express PD-L1. In some embodiments of any of the methods provided herein, a sample from an individual, e.g., an individual having a cancer of the disclosure, is determined to be PD-L1 positive if at least about 1% of tumor cells in the sample express PD-L1. In some embodiments of any of the methods provided herein, a sample from an individual, e.g., an individual having a cancer of the disclosure, is determined to be PD-L1 low positive if between about 1% and about 49% of tumor cells in the sample express PD-L1. In some embodiments of any of the methods provided herein, a sample from an individual, e.g., an individual having a cancer of the disclosure, is determined to be PD-L1 high positive if at least about 50% or more of tumor cells in the sample express PD-L1. In some embodiments of any of the methods provided herein, a sample from an individual, e.g., an individual having a cancer of the disclosure, is determined to be PD-L1 positive if the sample is PD-L1 low positive or PD-L1 high positive.

In some embodiments, the level of PD-L1 protein expression is measured using an immunohistochemistry assay. In some embodiments, the level of PD-L1 protein expression is measured using a VENTANA PD-L1 assay (SP142). In some embodiments, the level of PD-L1 protein expression is determined based on PD-L1 expression in tumor infiltrating immune cells (ICs) and/or tumor cells (TCs). Additional information about the VENTANA SP142 assay may be found in the website: www[dot]accessdata[dot]fda[dot]gov/cdrh_docs/pdf16/P160002c.pdf.

In some embodiments, the level of PD-L1 protein expression is determined based on PD-L1 tumor cell expression using an immunohistochemistry assay, such as a DAKO 22C3 assay. In some embodiments, the level of PD-L1 protein expression is assessed based on a tumor proportion score (TPS). The TPS is the percentage of tumor cells showing partial or complete PD-L1 membrane staining (e.g., at a ≥1+ intensity on a 0, 1+, 2+, and 3 scale) relative to all tumor cells present in the sample. In some embodiments, the TPS is calculated as: the number of PD-L1-positive tumor cells/Total number of PD-L1-positive tumor cells+Total number of PD-L1-negative tumor cells. As used herein, a PD-L1 low positive status is defined as a TPS of between 1% and 49%, and PD-L1 high positive status is defined as a TPS of 50% or greater. As used herein, a PD-L1 negative status is defined as a TPS of less than 1%. In some embodiments, a cancer of the disclosure is determined to be PD-L1 positive if it has PD-L1 low positive status or a PD-L1 high positive status. In some embodiments, a cancer of the disclosure is PD-L1 positive (e.g., the cancer is determined have a TPS of any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%, in a sample obtained from an individual having the cancer). In some embodiments, a cancer of the disclosure is PD-L1 low positive (e.g., the cancer is determined have a TPS of any of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, or about 49%, in a sample obtained from an individual having the cancer). In some embodiments, a cancer of the disclosure is PD-L1 high positive (e.g., the cancer is determined have a TPS of any of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%, in a sample obtained from an individual having the cancer). In some embodiments, a cancer of the disclosure is PD-L1 negative (e.g., the cancer is determined have a TPS of less than 1%, in a sample obtained from an individual having the cancer). Additional information about the DAKO 22C3 assay and the TPS score may be found, e.g., in the website: www.agilent.com/cs/library/usermanuals/public/29158_pd-11-ihc-22C3-pharmdx-nsclc-interpretation-manual.pdf.

### Antibodies

Provided herein are antibodies or antibody fragments that specifically bind to a PD-L1 polypeptide encoded by a CD274 gene, e.g., comprising one or more mutations described herein, or a portion thereof. The antibody may be of any suitable type of antibody, including, but not limited to, a monoclonal antibody, a polyclonal antibody, a multi-specific antibody (e.g., a bispecific antibody), or an antibody fragment, so long as the antibody or antibody fragment exhibits a specific antigen binding activity (e.g., binding to a PD-L1 polypeptide encoded by a CD274 gene, e.g., comprising one or more mutations described herein, or a portion thereof).

In some embodiments, a PD-L1 polypeptide encoded by a CD274 gene, e.g., comprising one or more mutations described herein, or a fragment thereof, is used as an immunogen to generate one or more antibodies of the disclosure, e.g., using standard techniques for polyclonal and monoclonal antibody preparation. In some embodiments, a PD-L1 polypeptide encoded by a CD274 gene, e.g., comprising one or more mutations described herein, or a fragment thereof, is used to provide antigenic peptide fragments (e.g., comprising any of at least about 8, at least about 10, at least about 15, at least about 20, at least about 30 or more amino acids) for use as immunogens to generate one or more antibodies of the disclosure, e.g., using standard techniques for polyclonal and monoclonal antibody preparation. As is known in the art, an antibody of the disclosure may be prepared by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptides, e.g., a PD-L1 polypeptide described herein, or a fragment thereof. The preparation can further include an adjuvant, such as Freund's complete or incomplete adjuvant, or a similar immunostimulatory agent.

In some embodiments, an antibody provided herein is a polyclonal antibody. Methods of producing polyclonal antibodies are known in the art. In some embodiments, an antibody provided herein is a monoclonal antibody, wherein a population of the antibody molecules contain only one species of an antigen binding site capable of immunoreacting or binding with a particular epitope, e.g., an epitope on a PD-L1 polypeptide provided herein. Methods of preparation of monoclonal antibodies are known in the art, e.g., using standard hybridoma techniques originally described by Kohler and Milstein (1975) Nature 256:495-497, human B cell hybridoma techniques (see Kozbor et al., 1983, Immunol. Today 4:72), EBV-hybridoma techniques (see Cole et al., pp. 77-96 In Monoclonal Antibodies and Cancer Therapy, Alan R. Liss, Inc., 1985), or trioma techniques. The technology for producing hybridomas is well known (see generally Current Protocols in Immunology, Coligan et al. ed., John Wiley & Sons, New York, 1994). A monoclonal antibody of the disclosure may also be identified and isolated by screening a recombinant combinatorial immunoglobulin library (e.g., an antibody phage display library) with the polypeptide of interest, e.g., a PD-L1 polypeptide provided herein or a fragment thereof. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; and Griffiths et al. (1993) EMBO J. 12:725-734. In some embodiments, monoclonal antibodies of the disclosure are recombinant antibodies, such as chimeric and humanized monoclonal antibodies, comprising both human and non-human portions. Such chimeric and/or humanized monoclonal antibodies can be produced by recombinant DNA techniques known in the art, for example, using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060. In some embodiments, a monoclonal antibody of the disclosure is a human monoclonal antibody. In some embodiments, human monoclonal antibodies are prepared using methods known in the art, e.g., using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93. For a detailed discussion of this technology for producing human antibodies and human monoclonal antibodies, and protocols for producing such antibodies, see, e.g., U.S. Pat. Nos. 5,625,126; 5,633,425; 5,569,825; 5,661,016; and 5,545,806.

In some embodiments, the antibody or antibody fragment of the disclosure is an isolated antibody or antibody fragment, which has been separated from a component of its natural environment or a cell culture used to produce the antibody or antibody fragment. In some embodiments, an antibody of the disclosure is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods.

In some embodiments, an antibody of the disclosure can be used to isolate a PD-L1 polypeptide provided herein, or a fragment thereof, by standard techniques, such as affinity chromatography or immunoprecipitation. In some embodiments, an antibody of the disclosure can be used to detect a PD-L1 polypeptide provided herein, or a fragment thereof, e.g., in a tissue sample, cellular lysate, or cell supernatant, in order to evaluate the level and/or pattern of expression of the PD-L1 polypeptide. Detection can be facilitated by coupling the antibody to a detectable substance. Thus, in some embodiments, an antibody of the disclosure is coupled to a detectable substance, such as enzymes, prosthetic groups, fluorescent materials, luminescent materials, bioluminescent materials, and radioactive materials. Non-limiting examples of suitable enzymes include, e.g., horseradish peroxidase, alkaline phosphatase, β-galactosidase, or acetylcholinesterase; examples of suitable prosthetic group complexes include, e.g., streptavidin/biotin and avidin/biotin; examples of suitable fluorescent materials include, e.g., umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, e.g., luciferase, luciferin, and aequorin; and examples of suitable radioactive materials include, e.g., ¹²⁵I, ¹³¹I, ³⁵S or ³H.

An antibody or antibody fragment of the disclosure may also be used diagnostically, e.g., to detect and/or monitor protein levels (e.g., protein levels of a PD-L1 polypeptide provided herein, or a fragment thereof) in tissues or body fluids (e.g., in a tumor cell-containing tissue or body fluid), e.g., according to the methods provided herein.

In certain embodiments, an antibody provided herein has a dissociation constant (Kd) of ≤1 μM, ≤100 nM, ≤10 nM, ≤1 nM, ≤0.1 nM, 0.01 nM, or 0.001 nM (e.g., 10⁻⁸ M or less, e.g., from 10⁻⁸M to 10⁻¹³ M, e.g., from 10⁻⁹ M to 10⁻¹³ M). Methods of measuring antibody affinity (e.g., Kd) are known in the art, and include, without limitation, a radiolabeled antigen binding assay (RIA) and a BIACORE® surface plasmon resonance assay. In some embodiments, antibody affinity (e.g., Kd) is determined using the Fab version of an antibody of the disclosure and its antigen (e.g., a PD-L1 polypeptide provided herein, or a fragment thereof). In some embodiments, a RIA is performed with the Fab version of an antibody of the disclosure and its antigen (e.g., a PD-L1 polypeptide provided herein, or a fragment thereof).

In certain embodiments, an antibody provided herein is an antibody fragment. Antibody fragments include, but are not limited to, Fab, Fab′, Fab′-SH, F(ab′)₂, Fv, and single-chain antibody molecules (e.g., scFv) fragments, and other fragments described herein.

In certain embodiments, an antibody provided herein is a diabody. Diabodies are antibody fragments with two antigen-binding sites that may be bivalent or bispecific. In certain embodiments, an antibody provided herein is a triabody or a tetrabody.

In certain embodiments, an antibody provided herein is a single-domain antibody. Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody, as well as production by recombinant host cells (e.g., E. coli or phage), as known in the art and as described herein.

In certain embodiments, an antibody provided herein is a chimeric antibody. In one example, a chimeric antibody comprises a non-human variable region (e.g., a variable region derived from a mouse, rat, hamster, rabbit, or non-human primate, such as a monkey), and a human constant region. In a further example, a chimeric antibody is a “class switched” antibody, in which the class or subclass of the antibody has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof), are derived from a non-human antibody, and framework regions (FRs) (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity. Humanized antibodies and methods of making them are known in the art. Human framework regions that may be used for humanization include but are not limited to: framework regions selected using the “best-fit” method; framework regions derived from the consensus sequence of human antibodies of a particular subgroup of light or heavy chain variable regions; human mature (somatically mutated) framework regions or human germline framework regions; and framework regions derived from screening FR libraries.

In certain embodiments, an antibody provided herein is a human antibody. Human antibodies can be produced using various techniques known in the art. For example, human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or are present extrachromosomally or integrated randomly into the animal's chromosomes. In such transgenic animals, e.g., mice, the endogenous immunoglobulin loci have generally been inactivated. Human variable regions from intact antibodies generated by such animals may be further modified, e.g., by combining with a different human constant region. Human antibodies can also be made by hybridoma-based methods known in the art, e.g., using known human myeloma and mouse-human heteromyeloma cell lines for the production of human monoclonal antibodies. Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Antibodies of the disclosure may be isolated by screening combinatorial libraries for antibodies with the desired activity or activities. For example, a variety of methods are known in the art for generating phage display libraries and screening such libraries for antibodies possessing the desired binding characteristics. In certain phage display methods, repertoires of VH and VL genes are separately cloned by polymerase chain reaction (PCR) and recombined randomly in phage libraries, which can then be screened for antigen-binding phage. Phage typically display antibody fragments, either as single-chain Fv (scFv) fragments or as Fab fragments. Libraries from immunized sources provide high-affinity antibodies to the immunogen without the requirement of constructing hybridomas. Alternatively, a naive antibody repertoire can be cloned (e.g., from human) to provide a single source of antibodies to a wide range of non-self and also self antigens without any immunization. Naive libraries can also be made synthetically by cloning un-rearranged V-gene segments from stem cells, and using PCR primers containing random sequences to amplify the highly variable CDR3 regions and to accomplish rearrangement in vitro. Antibodies or antibody fragments isolated from human antibody libraries are considered human antibodies or human antibody fragments herein.

In certain embodiments, an antibody provided herein is a multispecific antibody, e.g., a bispecific antibody. Multispecific antibodies are monoclonal antibodies that have binding specificities for at least two different sites or at least two different antigens. For example, one of the binding specificities can be to an immune checkpoint protein of the present disclosure, and the other can be to any other antigen, e.g., a PD-L1 polypeptide provided herein, or a fragment thereof. Multispecific antibodies can be prepared as full length antibodies or as antibody fragments. Techniques for making multispecific antibodies are known in the art and include, but are not limited to, recombinant co-expression of two immunoglobulin heavy chain-light chain pairs having different specificities, and “knob-in-hole” engineering. Multispecific antibodies may also be made by engineering electrostatic steering effects (e.g., by introducing mutations in the constant region) for making heterodimeric Fes; cross-linking two or more antibodies or fragments; using leucine zippers to produce bispecific antibodies; using “diabody” technology for making bispecific antibody fragments; using single-chain Fv (scFv) dimers; and preparing trispecific antibodies. Engineered antibodies with three or more functional antigen binding sites, including “Octopus antibodies,” are also included in the disclosure. Antibodies or antibody fragments of the disclosure also include “Dual Acting FAbs” or “DAF,” e.g., comprising an antigen binding site that binds to an immune checkpoint protein as well as another, different antigen.

In certain embodiments, amino acid sequence variants of the antibodies provided herein are contemplated. For example, it may be desirable to improve the binding affinity and/or other biological properties of the antibody. Amino acid sequence variants of an antibody of the disclosure may be prepared by introducing appropriate modifications into the nucleotide sequence encoding the antibody, or by peptide synthesis. Such modifications include, for example, deletions, and/or insertions, and/or substitutions of residues within the amino acid sequences of the antibody. Any combination of deletions, insertions, and substitutions can be made to arrive at the final antibody, provided that the final antibody possesses the desired characteristics, e.g., antigen-binding.

In certain embodiments, antibody variants having one or more amino acid substitutions are provided. Sites of interest for substitutional mutagenesis include the HVRs and FRs. Amino acid substitutions may be introduced into an antibody of interest, and the products may be screened for a desired activity, e.g., retained/improved antigen binding, decreased immunogenicity, or improved or reduced antibody-dependent cell-mediated cytotoxicity (ADCC) and/or complement-dependent cytotoxicity (CDC).

In certain embodiments, an antibody of the present disclosure is altered to increase or to decrease the extent to which the antibody is glycosylated. Addition or deletion of glycosylation sites to an antibody may be conveniently accomplished by altering the amino acid sequence of the antibody, such that one or more glycosylation sites is created or removed. Antibody variants having bisected oligosaccharides are further provided, e.g., in which a biantennary oligosaccharide attached to the Fc region of the antibody is bisected by GlcNAc. In some embodiments, antibody variants of the disclosure may have increased fucosylation. In some embodiments, antibody variants of the disclosure may have reduced fucosylation. In some embodiments, antibody variants of the disclosure may have improved ADCC function. In some embodiments, antibody variants of the disclosure may have decreased ADCC function. Antibody variants with at least one galactose residue in the oligosaccharide attached to the Fc region are also provided. Such antibody variants may have improved CDC function. In some embodiments, antibody variants of the disclosure may have increased CDC function. In some embodiments, antibody variants of the disclosure may have decreased CDC function.

In certain embodiments, one or more amino acid modifications may be introduced into the Fc region of an antibody of the present disclosure, thereby generating an Fe region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody variant that possesses some but not all effector functions, which make it a desirable candidate for applications in which the half-life of the antibody in vivo is important, yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc-gamma-R binding (hence likely lacking ADCC activity), but retains FcRn binding ability. The primary cells that mediate ADCC, e.g., NK cells, express Fc-gamma-RIII only, whereas monocytes express Fc-gamma-RI, Fc-gamma-RII and Fc-gamma-RIII. Antibodies with reduced effector function include those with substitution of one or more of Fc region residues 238, 265, 269, 270, 297, 327 and 329. Such Fc mutants include Fc mutants with substitutions at two or more of amino acid positions 265, 269, 270, 297 and 327, including the so-called “DANA” Fc mutant with substitutions of residues 265 and 297 to alanine. Antibody variants with improved or diminished binding to FcRs are also included in the disclosure. In certain embodiments, an antibody variant comprises an Fc region with one or more amino acid substitutions that improve ADCC, e.g., substitutions at positions 298, 333, and/or 334 of the Fc region. In some embodiments, numbering of Fc region residues is according to EU numbering of residues. In some embodiments, alterations are made in the Fc region that result in altered (i.e., either improved or diminished) C1q binding and/or CDC. In some embodiments, antibodies of the disclosure include antibodies with increased half-lives and improved binding to the neonatal Fc receptor (FcRn), e.g., comprising one or more substitutions that improve binding of the Fe region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434. See, also, Duncan & Winter, Nature 322:738-40 (1988); U.S. Pat. Nos. 5,648,260; 5,624,821; and WO 94/29351 for other examples of Fc region variants.

In certain embodiments, an antibody provided herein is a cysteine-engineered antibody, e.g., “thioMAb,” in which one or more residues of the antibody are substituted with cysteine residues. In some embodiments, the substituted residues occur at accessible sites of the antibody. By substituting those residues with cysteine, reactive thiol groups are thereby positioned at accessible sites of the antibody, and may be used to conjugate the antibody to other moieties, such as drug moieties or linker-drug moieties, e.g., to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine-engineered antibodies may be generated using any suitable method known in the art.

In some embodiments, an antibody or antibody fragment provided herein comprises a label or a tag. In some embodiments, the label or tag is a radiolabel, a fluorescent label, an enzymatic label, a sequence tag, biotin, or other ligands. Examples of labels or tags include, but are not limited to, 6×His-tag, biotin-tag, Glutathione-S-transferase (GST)-tag, green fluorescent protein (GFP)-tag, c-myc-tag, FLAG-tag, Thioredoxin-tag, Glu-tag, Nus-tag, V5-tag, calmodulin-binding protein (CBP)-tag, Maltose binding protein (MBP)-tag, Chitin-tag, alkaline phosphatase (AP)-tag, HRP-tag, Biotin Caboxyl Carrier Protein (BCCP)-tag, Calmodulin-tag, S-tag, Strep-tag, haemoglutinin (HA)-tag, digoxigenin (DIG)-tag, DsRed, RFP, Luciferase, Short Tetracysteine Tags, Halo-tag, and Nus-tag. In some embodiments, the label or tag comprises a detection agent, such as a fluorescent molecule or an affinity reagent or tag.

In some embodiments, an antibody or antibody fragment provided herein is conjugated to a drug molecule, e.g., an anti-cancer agent described herein, or a cytotoxic agent such as mertansine or monomethyl auristatin E (MMAE).

In certain embodiments, an antibody or antibody fragment provided herein may be further modified to contain additional nonproteinaceous moieties. Such moieties may be suitable for derivatization of the antibody, e.g., including but not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyamino acids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, polyethylene glycol propionaldehyde, and mixtures thereof. The polymers may be of any molecular weight, and may be branched or unbranched. The number of polymers attached to the antibody may vary, and if more than one polymer is attached, the polymers can be the same or different molecules. In general, the number and/or type of polymers used for derivatization can be determined based on considerations including, but not limited to, the particular properties or functions of the antibody to be improved, or whether the antibody derivative will be used in a therapy under defined conditions. In some embodiments, provided herein are antibodies conjugated to carbon nanotubes, e.g., for use in methods to selectively heat the antibody using radiation to a temperature at which cells proximal to the antibody are killed.

### Reporting

In some embodiments, the methods provided herein comprise generating a report, and/or providing a report to party.

In some embodiments, a report according to the present disclosure comprises information about one or more of: one or more mutations in a CD274 gene described herein (e.g., in a nucleic acid molecule comprising or encoding the CD274 gene, or a portion thereof, or in a PD-L1 polypeptide encoded by the CD274 gene, or a portion thereof); a cancer of the disclosure, e.g., comprising one or more mutations in a CD274 gene described herein (e.g., in a nucleic acid molecule comprising or encoding the CD274 gene, or a portion thereof, or in a PD-L1 polypeptide encoded by the CD274 gene, or a portion thereof); or a treatment, a therapy, or one or more treatment options for an individual having a cancer, such as a cancer of the disclosure (e.g., comprising one or more mutations in a CD274 gene described herein (e.g., in a nucleic acid molecule comprising or encoding the CD274 gene, or a portion thereof, or in a PD-L1 polypeptide encoded by the CD274 gene, or a portion thereof)).

In some embodiments, a report according to the present disclosure comprises information about the presence or absence of one or more mutations in a CD274 gene described herein (e.g., in a nucleic acid molecule comprising or encoding the CD274 gene, or a portion thereof, or in a PD-L1 polypeptide encoded by the CD274 gene, or a portion thereof) in a sample obtained from an individual, such as an individual having a cancer, e.g., a cancer provided herein. In one embodiment, a report according to the present disclosure indicates that one or more mutations in a CD274 gene described herein (e.g., in a nucleic acid molecule comprising or encoding the CD274 gene, or a portion thereof, or in a PD-L1 polypeptide encoded by the CD274 gene, or a portion thereof) are present in a sample obtained from the individual. In one embodiment, a report according to the present disclosure indicates that one or more mutations in a CD274 gene described herein (e.g., in a nucleic acid molecule comprising or encoding the CD274 gene, or a portion thereof, or in a PD-L1 polypeptide encoded by the CD274 gene, or a portion thereof) are not present in a sample obtained from the individual. In one embodiment, a report according to the present disclosure indicates that one or more mutations in a CD274 gene described herein (e.g., in a nucleic acid molecule comprising or encoding the CD274 gene, or a portion thereof, or in a PD-L1 polypeptide encoded by the CD274 gene, or a portion thereof) have been detected in a sample obtained from the individual. In one embodiment, a report according to the present disclosure indicates that one or more mutations in a CD274 gene described herein (e.g., in a nucleic acid molecule comprising or encoding the CD274 gene, or a portion thereof, or in a PD-L1 polypeptide encoded by the CD274 gene, or a portion thereof) have not been detected in a sample obtained from the individual. In some embodiments, the report comprises an identifier for the individual from which the sample was obtained.

In some embodiments, the report includes information on the role of one or more mutations in a CD274 gene described herein (e.g., in a nucleic acid molecule comprising or encoding the CD274 gene, or a portion thereof, or in a PD-L1 polypeptide encoded by the CD274 gene, or a portion thereof), or the wild type counterparts (e.g., a wild type CD274 gene or a wild type PD-L1 polypeptide), in disease, such as in cancer. Such information can include one or more of: information on prognosis of a cancer, such as a cancer provided herein, e.g., comprising one or more mutations in a CD274 gene described herein (e.g., in a nucleic acid molecule comprising or encoding the CD274 gene, or a portion thereof, or in a PD-L1 polypeptide encoded by the CD274 gene, or a portion thereof); information on resistance of a cancer, such as a cancer provided herein, e.g., comprising one or more mutations in a CD274 gene described herein (e.g., in a nucleic acid molecule comprising or encoding the CD274 gene, or a portion thereof, or in a PD-L1 polypeptide encoded by the CD274 gene, or a portion thereof), to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having a cancer, such as a cancer provided herein, e.g., comprising one or more mutations in a CD274 gene described herein (e.g., in a nucleic acid molecule comprising or encoding the CD274 gene, or a portion thereof, or in a PD-L1 polypeptide encoded by the CD274 gene, or a portion thereof) and identified in the report. In some embodiments, the report includes information or a recommendation on the administration of a treatment (e.g., an anti-cancer therapy provided herein, or a treatment selected or identified according to the methods provided herein). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein; detecting one or more mutations in a CD274 gene described herein (e.g., in a nucleic acid molecule comprising or encoding the CD274 gene, or a portion thereof, or in a PD-L1 polypeptide encoded by the CD274 gene, or a portion thereof) in the sample, or acquiring knowledge of the presence of one or more mutations in a CD274 gene described herein (e.g., in a nucleic acid molecule comprising or encoding the CD274 gene, or a portion thereof, or in a PD-L1 polypeptide encoded by the CD274 gene, or a portion thereof) in the sample; and generating a report. In some embodiments, a report generated according to the methods provided herein comprises one or more of: information about the presence or absence of one or more mutations in a CD274 gene described herein (e.g., in a nucleic acid molecule comprising or encoding the CD274 gene, or a portion thereof, or in a PD-L1 polypeptide encoded by the CD274 gene, or a portion thereof) in the sample; an identifier for the individual from which the sample was obtained; information on the role of the one or more mutations in a CD274 gene described herein (e.g., in a nucleic acid molecule comprising or encoding the CD274 gene, or a portion thereof, or in a PD-L1 polypeptide encoded by the CD274 gene, or a portion thereof), or wild type counterparts (e.g., a wild type CD274 gene or a wild type PD-L1 polypeptide), in disease (e.g., such as in cancer); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, the report generated is a personalized cancer report.

A report according to the present disclosure may be in an electronic, web-based, or paper form. The report may be provided to an individual or a patient (e.g., an individual or a patient with a cancer, such as a cancer provided herein, e.g., comprising one or more mutations in a CD274 gene described herein (e.g., in a nucleic acid molecule comprising or encoding the CD274 gene, or a portion thereof, or in a PD-L1 polypeptide encoded by the CD274 gene, or a portion thereof)), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting one or more mutations in a CD274 gene described herein (e.g., in a nucleic acid molecule comprising or encoding the CD274 gene, or a portion thereof, or in a PD-L1 polypeptide encoded by the CD274 gene, or a portion thereof) in a sample obtained from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of one or more mutations in a CD274 gene described herein (e.g., in a nucleic acid molecule comprising or encoding the CD274 gene, or a portion thereof, or in a PD-L1 polypeptide encoded by the CD274 gene, or a portion thereof) in a sample obtained from an individual (e.g., an individual having a cancer).

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### Software, Systems, and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform the method according to any of the embodiments described herein.

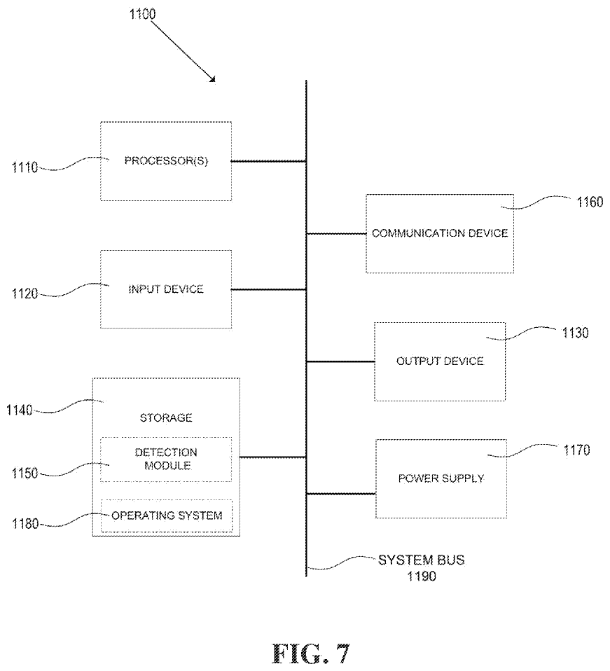

FIG. 7 illustrates an example of a computing device in accordance with one embodiment. Device 1100 can be a host computer connected to a network. Device 1100 can be a client computer or a server. As shown in FIG. 7, device 1100 can be any suitable type of microprocessor-based device, such as a personal computer, workstation, server or handheld computing device (portable electronic device) such as a phone or tablet. The device can include, for example, one or more of processor(s) 1110, input device 1120, output device 1130, storage 1140, communication device 1160, power supply 1170, operating system 1180, and system bus 1190. Input device 1120 and output device 1130 can generally correspond to those described herein, and can either be connectable or integrated with the computer.

Input device 1120 can be any suitable device that provides input, such as a touch screen, keyboard or keypad, mouse, or voice-recognition device. Output device 1130 can be any suitable device that provides output, such as a touch screen, haptics device, or speaker.

Storage 1140 can be any suitable device that provides storage (e.g., an electrical, magnetic or optical memory including a RAM (volatile and non-volatile), cache, hard drive, or removable storage disk). Communication device 1160 can include any suitable device capable of transmitting and receiving signals over a network, such as a network interface chip or device. The components of the computer can be connected in any suitable manner, such as via a wired media (e.g., a physical bus, ethernet, or any other wire transfer technology) or wirelessly (e.g., Bluetooth®, Wi-Fi®, or any other wireless technology). For example, in FIG. 7, the components are connected by System Bus 1190.

Detection module 1150, which can be stored as executable instructions in storage 1140 and executed by processor(s) 1110, can include, for example, the processes that embody the functionality of the present disclosure (e.g., as embodied in the devices as described herein).

Detection module 1150 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 1140, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Detection module 1150 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

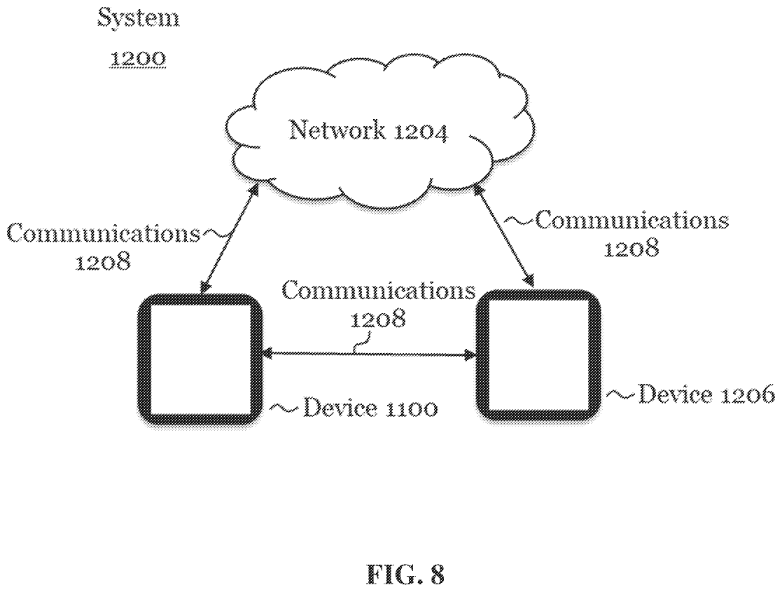

Device 1100 may be connected to a network (e.g., Network 1204, as shown in FIG. 8 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1100 can implement any operating system (e.g., Operating System 1180) suitable for operating on the network. Detection module 1150 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, Operating System 1180 is executed by one or more processors, e.g., Processor(s) 1110.

Device 1100 can further include Power Supply 1170, which can be any suitable power supply.

FIG. 8 illustrates an example of a computing system in accordance with one embodiment. In System 1200, Device 1100 (e.g., as described above and illustrated in FIG. 7) is connected to Network 1204, which is also connected to Device 1206. In some embodiments, Device 1206 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system. Devices 1100 and 1206 may communicate, e.g., using suitable communication interfaces via Network 1204, such as a Local Area Network (LAN), Virtual Private Network (VPN), or the Internet. In some embodiments, Network 1204 can be, for example, the Internet, an intranet, a virtual private network, a cloud network, a wired network, or a wireless network. Devices 1100 and 1206 may communicate, in part or in whole, via wireless or hardwired communications, such as Ethernet, IEEE 802.11b wireless, or the like. Additionally, Devices 1100 and 1206 may communicate, e.g., using suitable communication interfaces, via a second network, such as a mobile/cellular network. Communication between Devices 1100 and 1206 may further include or communicate with various servers such as a mail server, mobile server, media server, telephone server, and the like. In some embodiments, Devices 1100 and 1206 can communicate directly (instead of, or in addition to, communicating via Network 1204), e.g., via wireless or hardwired communications, such as Ethernet, IEEE 802.11b wireless, or the like. In some embodiments, Devices 1100 and 1206 communicate via Communications 1208, which can be a direct connection or can occur via a network (e.g., Network 1204).

One or all of Devices 1100 and 1206 generally include logic (e.g., http web server logic) or is programmed to format data, accessed from local or remote databases or other sources of data and content, for providing and/or receiving information via Network 1204 according to various examples described herein.

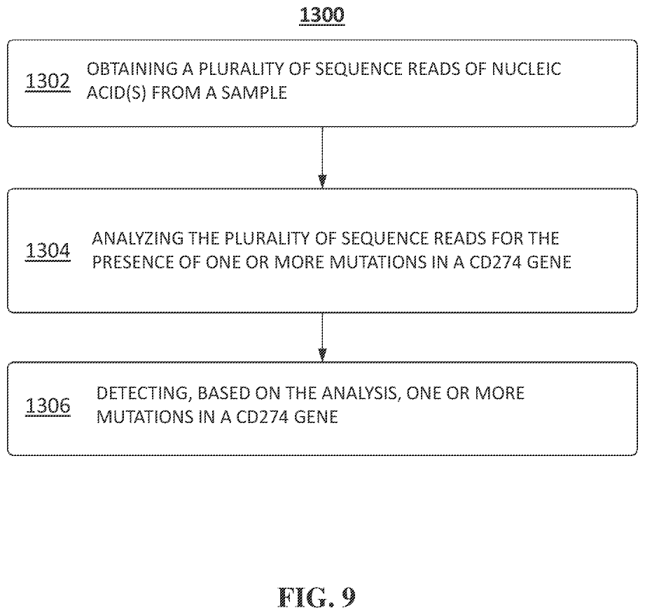

FIG. 9 illustrates an exemplary process 1300 for detecting one or more mutations in a CD274 gene, or a portion thereof, in accordance with some embodiments. Process 1300 is performed, for example, using one or more electronic devices implementing a software program. In some examples, process 1300 is performed using a client-server system, and the blocks of process 1300 are divided up in any manner between the server and a client device. In other examples, the blocks of process 1300 are divided up between the server and multiple client devices. Thus, while portions of process 1300 are described herein as being performed by particular devices of a client-server system, it will be appreciated that process 1300 is not so limited. In some embodiments, the executed steps can be executed across many systems, e.g., in a cloud environment. In other examples, process 1300 is performed using only a client device or only multiple client devices. In process 1300, some blocks are, optionally, combined, the order of some blocks is, optionally, changed, and some blocks are, optionally, omitted. In some examples, additional steps may be performed in combination with the process 1300. Accordingly, the operations as illustrated (and described in greater detail below) are exemplary by nature and, as such, should not be viewed as limiting.

At block 1302, a plurality of sequence reads of one or more nucleic acids is obtained, wherein the one or more nucleic acids are derived from a sample obtained from an individual. In some embodiments, the sample is obtained from an individual having a cancer, such as a cancer described herein. In some embodiments, the sequence reads are obtained using a sequencer, e.g., as described herein or otherwise known in the art. In some embodiments, the nucleic acid(s) comprise one or more nucleic acids corresponding to a CD274 gene of the present disclosure, or portion thereof. Optionally, prior to obtaining the sequence reads, the sample is purified, enriched (e.g., for nucleic acid(s) corresponding to a CD274 gene of the present disclosure, or portion thereof), and/or subjected to PCR amplification. At block 1304, an exemplary system (e.g., one or more electronic devices) analyzes the plurality of sequence reads for the presence of one or more mutations in a CD274 gene, or a portion thereof. At block 1306, the system detects (e.g., based on the analysis) one or more mutations in a CD274 gene, or a portion thereof, in the sample.

### Anti-Cancer Therapies

Certain aspects of the present disclosure relate to anti-cancer therapies, as well as methods for identifying an individual who may benefit from treatment with an anti-cancer therapy, methods for selecting an anti-cancer therapy for treating an individual, methods for identifying an anti-cancer therapy as a treatment option, methods for treating or delaying progression of cancer comprising administration of an anti-cancer therapy, uses for anti-cancer therapies (e.g., in methods of treating or delaying progression of cancer in an individual, or in methods for manufacturing a medicament for treating or delaying progression of cancer), and the like. These methods and uses are based, at least in part, on the observations of CD274 mutations in various cancer types, e.g., as described herein, as well as their association with changes in the levels of PD-L1 expression. Without wishing to be bound to theory, it is thought that these CD274 mutations can identify patients that would benefit from appropriate anti-cancer therapies such as one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof. Furthermore, without wishing to be bound to theory, it is thought that these CD274 mutations can identify patients that would benefit from an anti-cancer therapy other than an immune checkpoint inhibitor.

In some embodiments, the anti-cancer therapy comprises a cyclin-dependent kinase (CDK) inhibitor. In some embodiments, the CDK inhibitor inhibits CDK4. In some embodiments, the CDK inhibitor inhibits Cyclin D/CDK4. In some embodiments, the anti-cancer therapy/CDK inhibitor is (a) a small molecule that inhibits one or more enzymatic activities of CDK4, (b) an antibody that inhibits one or more activities of CDK4 (e.g., by binding to and inhibiting one or more activities of CDK4, binding to and inhibiting expression of CDK4, and/or binding to and inhibiting one or more activities of a cell expressing CDK4, such as by inducing antibody-dependent cellular cytotoxicity, ADCC, or phagocytosis, ADCP), or (c) a nucleic acid that inhibits expression of CDK4 (e.g., an antisense oligonucleotide, miRNA, siRNA, morpholino, CRISPR-based therapeutic, and the like). In some embodiments, the CDK inhibitor inhibits CDK4 and CDK6. In some embodiments, the CDK inhibitor is a small molecule inhibitor of CDK4 (e.g., a competitive or non-competitive inhibitor). Non-limiting examples of CDK inhibitors include palbociclib, ribociclib, and abemaciclib, as well as pharmaceutically acceptable salts thereof.

In some embodiments, the anti-cancer therapy comprises a murine double minute 2 homolog (MDM2) inhibitor. In some embodiments, the anti-cancer therapy/MDM2 inhibitor is (a) a small molecule that inhibits one or more activities of MDM2 (e.g., binding to p53), (b) an antibody that inhibits one or more activities of MDM2 (e.g., by binding to and inhibiting one or more activities of MDM2, binding to and inhibiting expression of MDM2, and/or binding to and inhibiting one or more activities of a cell expressing MDM2, such as by inducing antibody-dependent cellular cytotoxicity, ADCC, or phagocytosis, ADCP), or (c) a nucleic acid that inhibits expression of MDM2 (e.g., an antisense oligonucleotide, miRNA, siRNA, morpholino, CRISPR-based therapeutic, and the like). In some embodiments, the MDM2 inhibitor is a small molecule inhibitor of MDM2 (e.g., a competitive or non-competitive inhibitor). Non-limiting examples of MDM2 inhibitors include nutlin-3a, RG7112, idasanutlin (RG7388), AMG-232, MI-63, MI-291, MI-391, MI-77301 (SAR405838), APG-115, DS-3032b, NVP-CGM097, and HDM-201 (siremadlin), as well as pharmaceutically acceptable salts thereof. In some embodiments, the MDM2 inhibitor inhibits or disrupts interaction between MDM2 and p53.

In some embodiments, the anti-cancer therapy comprises one or more of an antimetabolite, DNA-damaging agent, or platinum-containing therapeutic (e.g., 5-azacitadine, 5-fluorouracil, acadesine, busulfan, carboplatin, cisplatin, chlorambucil, CPT-11, cytarabine, daunorubicin, decitabine, doxorubicin, etoposide, fludarabine, gemcitabine, idarubicin, radiation, oxaliplatin, temozolomide, topotecan, trabectedin, GSK2830371, or rucaparib); a pro-apoptotic agent (e.g., a BCL2 inhibitor or downregulator, SMAC mimetic, or TRAIL agonist such as ABT-263, ABT-737, oridonin, venetoclax, combination of venetoclax and an anti-CD20 antibody such as obinutuzumab or rituximab, 1396-11, ABT-10, SM-164, D269H/E195R, or rhTRAIL); a tyrosine kinase inhibitor (e.g., as described herein); an inhibitor of RAS, RAF, MEK, or the MAPK pathway (e.g., AZD6244, dabrafenib, LGX818, PD0325901, pimasertib, trametinib, or vemurafenib); an inhibitor of PI3K, mTOR, or Akt (e.g., as described herein); a CDK inhibitor (e.g., as described herein); a PKC inhibitor (e.g., LXS196 or sotrastaurin); an antibody-based therapeutic (e.g., an anti-PD-1 or anti-PDL1 antibody such as atezolizumab, pembrolizumab, nivolumab, or spartalizumab; an anti-CD20 antibody such as obinutuzumab or rituximab; or an anti-DR5 antibody such as drozitumab); a proteasome inhibitor (e.g., bortezomib, carfilzomib, ixazomib, or MG-132); an HDAC inhibitor (e.g., SAHA or VPA); an antibiotic (e.g., actinomycin D); a zinc-containing therapeutic (e.g., zinc or ZMC1); an HSP inhibitor (e.g., geldanamycin); an ATPase inhibitor (e.g., archazolid); a mitotic inhibitor (e.g., paclitaxel or vincristine); metformin; methotrexate; tanshinone IIA; and/or P5091.

In some embodiments, the anti-cancer therapy comprises a tyrosine kinase inhibitor. In some embodiments, the anti-cancer therapy/tyrosine kinase inhibitor is (a) a small molecule that inhibits one or more enzymatic activities of a tyrosine kinase, (b) an antibody that inhibits one or more activities of a tyrosine kinase (e.g., by binding to and inhibiting one or more activities of the tyrosine kinase, binding to and inhibiting expression, such as cell surface expression, of the tyrosine kinase, and/or binding to and inhibiting one or more activities of a cell expressing the tyrosine kinase, such as by inducing antibody-dependent cellular cytotoxicity, ADCC, or phagocytosis, ADCP), or (c) a nucleic acid that inhibits expression of a tyrosine kinase (e.g., an antisense oligonucleotide, miRNA, siRNA, morpholino, CRISPR-based therapeutic, and the like). In some embodiments, the tyrosine kinase inhibitor is a small molecule inhibitor of a tyrosine kinase (e.g., a competitive or non-competitive inhibitor). Non-limiting examples of tyrosine kinase inhibitors include imatinib, crenolanib, linifanib, ninetedanib, axitinib, dasatinib, imetelstat, midostaurin, pazopanib, sorafenib, sunitinb, motesanib, masitinib, vatalanib, cabozanitinib, tivozanib, OSI-930, Ki8751, telatinib, dovitinib, tyrphostin AG 1296, and amuvatinib, as well as pharmaceutically acceptable salts thereof.

In some embodiments, the anti-cancer therapy comprises a mitogen-activated protein kinase (MEK) inhibitor. In some embodiments, the MEK inhibitor inhibits one or more activities of MEK1 and/or MEK2. In some embodiments, the anti-cancer therapy/MEK inhibitor is (a) a small molecule that inhibits one or more enzymatic activities of MEK, (b) an antibody that inhibits one or more activities of MEK (e.g., by binding to and inhibiting one or more activities of MEK, binding to and inhibiting expression of MEK, and/or binding to and inhibiting one or more activities of a cell expressing MEK, such as by inducing antibody-dependent cellular cytotoxicity, ADCC, or phagocytosis, ADCP), or (c) a nucleic acid that inhibits expression of MEK (e.g., an antisense oligonucleotide, miRNA, siRNA, morpholino, CRISPR-based therapeutic, and the like). In some embodiments, the MEK inhibitor is a small molecule inhibitor of MEK (e.g., a competitive or non-competitive inhibitor). Non-limiting examples of MEK inhibitors include trametinib, cobimetinib, binimetinib, CI-1040, PD0325901, selumetinib, AZD8330, TAK-733, GDC-0623, refametinib, pimasertib, R04987655, R05126766, WX-544, and HL-085, as well as pharmaceutically acceptable salts thereof. In some embodiments, the anti-cancer therapy inhibits one or more activities of the Raf/MEK/ERK pathway, including inhibitors of Raf, MEK, and/or ERK.

In some embodiments, the anti-cancer therapy comprises a mammalian target of rapamycin (mTOR) inhibitor. In some embodiments, the anti-cancer therapy/mTOR inhibitor is (a) a small molecule that inhibits one or more enzymatic activities of mTOR, (b) an antibody that inhibits one or more activities of mTOR (e.g., by binding to and inhibiting one or more activities of mTOR, binding to and inhibiting expression of mTOR, and/or binding to and inhibiting one or more activities of a cell expressing mTOR, such as by inducing antibody-dependent cellular cytotoxicity, ADCC, or phagocytosis, ADCP), or (c) a nucleic acid that inhibits expression of mTOR (e.g., an antisense oligonucleotide, miRNA, siRNA, morpholino, CRISPR-based therapeutic, and the like). In some embodiments, the mTOR inhibitor is a small molecule inhibitor of mTOR (e.g., a competitive inhibitor, such as an ATP-competitive inhibitor, or a non-competitive inhibitor, such as a rapamycin analog). Non-limiting examples of mTOR inhibitors include temsirolimus, everolimus, ridaforolimus, dactolisib, GSK2126458, XL765, AZD8055, AZD2014, MLN128, PP242, NVP-BEZ235, LY3023414, PQR309, PKI587, and OSI027, as well as pharmaceutically acceptable salts thereof. In some embodiments, the anti-cancer therapy inhibits one or more activities of the Akt/mTOR pathway, including inhibitors of Akt and/or mTOR.

In some embodiments, the anti-cancer therapy comprises a PI3K inhibitor or Akt inhibitor. In some embodiments, the PI3K inhibitor inhibits one or more activities of PI3K. In some embodiments, the anti-cancer therapy/PI3K inhibitor is (a) a small molecule that inhibits one or more enzymatic activities of PI3K, (b) an antibody that inhibits one or more activities of PI3K (e.g., by binding to and inhibiting one or more activities of PI3K, binding to and inhibiting expression of PI3K, and/or binding to and inhibiting one or more activities of a cell expressing PI3K, such as by inducing antibody-dependent cellular cytotoxicity, ADCC, or phagocytosis, ADCP), or (c) a nucleic acid that inhibits expression of PI3K (e.g., an antisense oligonucleotide, miRNA, siRNA, morpholino, CRISPR-based therapeutic, and the like). In some embodiments, the PI3K inhibitor is a small molecule inhibitor of PI3K (e.g., a competitive or non-competitive inhibitor). Non-limiting examples of PI3K inhibitors include GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDC0032, IPI145, INK1117 (MLN1117), SAR260301, KIN-193 (AZD6482), duvelisib, GS-9820, GSK2636771, GDC-0980, AMG319, pazobanib, and alpelisib (BYL719, Piqray), as well as pharmaceutically acceptable salts thereof. In some embodiments, the AKT inhibitor inhibits one or more activities of AKT (e.g., AKT1). In some embodiments, the anti-cancer therapy/AKT inhibitor is (a) a small molecule that inhibits one or more enzymatic activities of AKT1, (b) an antibody that inhibits one or more activities of AKT1 (e.g., by binding to and inhibiting one or more activities of AKT1, binding to and inhibiting expression of AKT1, and/or binding to and inhibiting one or more activities of a cell expressing AKT1, such as by inducing antibody-dependent cellular cytotoxicity, ADCC, or phagocytosis, ADCP), or (c) a nucleic acid that inhibits expression of AKT1 (e.g., an antisense oligonucleotide, miRNA, siRNA, morpholino, CRISPR-based therapeutic, and the like). In some embodiments, the AKT1 inhibitor is a small molecule inhibitor of AKT1 (e.g., a competitive or non-competitive inhibitor). Non-limiting examples of AKT1 inhibitors include GSK690693, GSK2141795 (uprosertib), GSK2110183 (afuresertib), AZD5363, GDC-0068 (ipatasertib), AT7867, CCT128930, MK-2206, BAY 1125976, AKT1 and AKT2-IN-1, perifosine, and VIII, as well as pharmaceutically acceptable salts thereof. In some embodiments, the AKT1 inhibitor is a pan-Akt inhibitor.

In some embodiments, the anti-cancer therapy is a hedgehog (Hh) inhibitor. In some embodiments, the anti-cancer therapy/Hh inhibitor is (a) a small molecule that inhibits one or more enzymatic activities of Hh, (b) an antibody that inhibits one or more activities of Hh (e.g., by binding to and inhibiting one or more activities of Hh, binding to and inhibiting expression of Hh, and/or binding to and inhibiting one or more activities of a cell expressing Hh, such as by inducing antibody-dependent cellular cytotoxicity, ADCC, or phagocytosis, ADCP), or (c) a nucleic acid that inhibits expression of Hh (e.g., an antisense oligonucleotide, miRNA, siRNA, morpholino, CRISPR-based therapeutic, and the like). In some embodiments, the Hh inhibitor is a small molecule inhibitor of Hh (e.g., a competitive or non-competitive inhibitor). Non-limiting examples of Hh inhibitors include sonidegib, vismodegib, erismodegib, saridegib, BMS833923, PF-04449913, and LY2940680, as well as pharmaceutically acceptable salts thereof.

In some embodiments, the anti-cancer therapy comprises a heat shock protein (HSP) inhibitor, a MYC inhibitor, an HDAC inhibitor, an immunotherapy, a neoantigen, a vaccine, or a cellular therapy.

In some embodiments, the anti-cancer therapy comprises one or more of an immune checkpoint inhibitor, a chemotherapy, a VEGF inhibitor, an Integrin β3 inhibitor, a statin, an EGFR inhibitor, an mTOR inhibitor, a PI3K inhibitor, a MAPK inhibitor, or a CDK4/6 inhibitor.

In some embodiments, the anti-cancer therapy comprises a kinase inhibitor. In some embodiments, the methods provided herein comprise administering to the individual a kinase inhibitor, e.g., in combination with another anti-cancer therapy. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). In some embodiments, the kinase inhibitor is an ALK kinase inhibitor, e.g., as described in examples 3-39 of WO2005016894, which is incorporated herein by reference.

In some embodiments, the anti-cancer therapy comprises a heat shock protein (HSP) inhibitor. In some embodiments, the methods provided herein comprise administering to the individual an HSP inhibitor, e.g., in combination with another anti-cancer therapy. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor. In some embodiments, the methods provided herein comprise administering to the individual a MYC inhibitor, e.g., in combination with another anti-cancer therapy. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor. In some embodiments, the methods provided herein comprise administering to the individual an HDAC inhibitor, e.g., in combination with another anti-cancer therapy. In some embodiments, the HDAC inhibitor is belinostat (PXD101, Beleodaq®), SAHA (vorinostat, suberoylanilide hydroxamine, Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPTOE028, BRD8430, MRLB-223, apicidin, RGFP966, BG45, PCI-34051, C149 (NCC149), TMP269, Cpd2, T247, T326, LMK235, CIA, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by Li et al., Cold Spring Harb Perspect Med (2016) 6(10):a026831, or PX117445.

In some embodiments, the anti-cancer therapy comprises a VEGF inhibitor. In some embodiments, the methods provided herein comprise administering to the individual a VEGF inhibitor, e.g., in combination with another anti-cancer therapy. In some embodiments, the VEGF inhibitor is Bevacizumab (Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin β3 inhibitor. In some embodiments, the methods provided herein comprise administering to the individual an integrin β3 inhibitor, e.g., in combination with another anti-cancer therapy. In some embodiments, the integrin β3 inhibitor is anti-avb3 (clone LM609), cilengitide (EMD121974, NSC, 707544), an siRNA, GLPG0187, MK-0429, CNT095, TN-161, etaracizumab (MEDI-522), intetumumab (CNT095) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an αIIbβ3 integrin inhibitor. In some embodiments, the methods provided herein comprise administering to the individual an αIIbβ3 integrin inhibitor, e.g., in combination with another anti-cancer therapy. In some embodiments, the αIIbβ3 integrin inhibitor is abciximab, eptifibatide (Integrilin®), or tirofiban (Aggrastat®).

In some embodiments, the anti-cancer therapy comprises a statin or a statin-based agent. In some embodiments, the methods provided herein comprise administering to the individual a statin or a statin-based agent, e.g., in combination with another anti-cancer therapy. In some embodiments, the statin or statin-based agent is simvastatin, atorvastatin, fluvastatin, pitavastatin, pravastatin, rosuvastatin, or cerivastatin.

In some embodiments, the anti-cancer therapy comprises a MAPK inhibitor. In some embodiments, the methods provided herein comprise administering to the individual a MAPK inhibitor, e.g., in combination with another anti-cancer therapy. In some embodiments, the MAPK inhibitor is SB203580, SKF-86002, BIRB-796, SC-409, RJW-67657, BIRB-796, VX-745, R03201195, SB-242235, or MW181.

In some embodiments, the anti-cancer therapy comprises an EGFR inhibitor. In some embodiments, the methods provided herein comprise administering to the individual an EGFR inhibitor, e.g., in combination with another anti-cancer therapy. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (ACO0010), rociletinib (CO-1686), BMS-690514, YH5448, PF-06747775, ASP8273, PF299804, AP26113, or erlotinib. In some embodiments, the EGFR inhibitor is gefitinib or cetuximab.

In some embodiments, the anti-cancer therapy comprises a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy. In some embodiments, the methods provided herein comprise administering to the individual a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy, e.g., in combination with another anti-cancer therapy. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen expressed by a cancer of the disclosure (e.g., a neoantigen corresponding to a CD274 nucleic acid molecule or polypeptide described herein). The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et ah, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21), 2013). In some embodiments, such cancer vaccines augment the anti-cancer response.

In some embodiments, the cancer vaccine comprises a polynucleotide that encodes a neoantigen, e.g., a neoantigen expressed by a cancer of the disclosure (e.g., a neoantigen corresponding to a CD274 nucleic acid molecule or polypeptide described herein). In some embodiments, the cancer vaccine comprises DNA that encodes a neoantigen, e.g., a neoantigen expressed by a cancer of the disclosure (e.g., a neoantigen corresponding to a CD274 nucleic acid molecule or polypeptide described herein). In some embodiments, the cancer vaccine comprises RNA that encodes a neoantigen, e.g., a neoantigen expressed by a cancer of the disclosure (e.g., a neoantigen corresponding to a CD274 nucleic acid molecule or polypeptide described herein). In some embodiments, the cancer vaccine comprises a polynucleotide that encodes a neoantigen, e.g., a neoantigen expressed by a cancer of the disclosure (e.g., a neoantigen corresponding to a CD274 nucleic acid molecule or polypeptide described herein). In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543). prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFa-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen, such as a neoantigen corresponding to a CD274 polypeptide provided herein.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen corresponding to a CD274 polypeptide provided herein.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen corresponding to a CD274 polypeptide provided herein, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure, e.g., an antigen corresponding to a CD274 polypeptide of the disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, the anti-cancer therapy comprises an immune checkpoint inhibitor. In some embodiments, the methods provided herein comprise administering to the individual an immune checkpoint inhibitor, e.g., in combination with another anti-cancer therapy. In some embodiments, the methods provided herein comprise administering to an individual an effective amount of an immune checkpoint inhibitor. In some embodiments, the methods provided herein comprise administering to the individual an anti-cancer therapy other than an immune checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-116, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B37-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H1,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist is a molecule that inhibits the binding of PD-1 to its ligand binding partners. In a specific embodiment, the PD-1 ligand binding partners are PD-L1 and/or PD-L2. In another instance, a PD-L1 binding antagonist is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is one or more of MDX-1 106 (nivolumab), MK-3475 (pembrolizumab, Keytruda®), MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANBO11), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.570, MPDL3280A (atezolizumab), MDX-1 105, MED14736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MED14736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the methods provided herein comprise selecting or administering an anti-cancer therapy other than a PD-1- or PD-L1-targeted immune checkpoint inhibitor (e.g., based on acquiring knowledge of or detecting one or more CD274 mutations of the disclosure in a cancer of the disclosure, or in a sample from an individual, e.g., having a cancer of the disclosure), but may comprise selecting or administering an immune checkpoint inhibitor that is not targeted to PD-1- or PD-L1. In some embodiments, the methods provided herein comprise selecting or administering an anti-cancer therapy other than a PD-L1/PD-1 axis-targeted immune checkpoint inhibitor (e.g., based on acquiring knowledge of or detecting one or more CD274 mutations of the disclosure in a cancer of the disclosure, or in a sample from an individual, e.g., having a cancer of the disclosure), but may comprise selecting or administering an immune checkpoint inhibitor that is not targeted to the PD-L1/PD-1 axis-targeted.

In some embodiments, the anti-cancer therapy comprises an immunoregulatory molecule or a cytokine. In some embodiments, the methods provided herein comprise administering to the individual an immunoregulatory molecule or a cytokine, e.g., in combination with another anti-cancer therapy. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3a (Lax), MIP-3p, Hcc-1, MPIF-1, MPIF-2, MCP-2, MCP-3, MCP-4, MCP-5, Eotaxin, Tarc, Elc, 1309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is monovalent and/or monospecific. In some embodiments, the immune checkpoint inhibitor is multivalent and/or multispecific.

In some embodiments, the anti-cancer therapy comprises an anti-cancer agent that inhibits expression of a nucleic acid that comprises or encodes a CD274 gene or a portion thereof comprising one or more mutations described herein, or a PD-L1 polypeptide encoded by a nucleic acid that comprises or encodes a CD274 gene or a portion thereof comprising one or more mutations described herein. In some embodiments, the methods provided herein comprise administering to the individual an anti-cancer agent that inhibits expression of a nucleic acid that comprises or encodes a CD274 gene or a portion thereof comprising one or more mutations described herein, or a PD-L1 polypeptide encoded by a nucleic acid that comprises or encodes a CD274 gene or a portion thereof comprising one or more mutations described herein, e.g., in combination with another anti-cancer therapy.

In some embodiments, the anti-cancer therapy comprises a nucleic acid molecule, such as a dsRNA, an siRNA, or an shRNA. In some embodiments, the methods provided herein comprise administering to the individual a nucleic acid molecule, such as a dsRNA, an siRNA, or an shRNA, e.g., in combination with another anti-cancer therapy. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. In some embodiments, a dsRNA, an siRNA, or an shRNA of the disclosure comprises a nucleotide sequence that is configured to hybridize to a nucleic acid that comprises or encodes a CD274 gene or a portion thereof comprising one or more mutations described herein. In some embodiments, a dsRNA, an siRNA, or an shRNA of the disclosure comprises a nucleotide sequence that is configured to hybridize to a nucleic acid that comprises or encodes a CD274 gene or a portion thereof comprising one or more mutations described herein, or a portion thereof. Methods for designing, optimizing, producing, and using dsRNAs, siRNAs, or shRNAs, are known in the art.

In some embodiments, the anti-cancer therapy comprises a chemotherapy. In some embodiments, the methods provided herein comprise administering to the individual a chemotherapy, e.g., in combination with another anti-cancer therapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein transferase inhibitors, transplatinum, and pharmaceutically acceptable salts, acids, or derivatives of any of the above.

Some non-limiting examples of chemotherapeutic drugs which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, the anti-cancer therapy comprises a kinase inhibitor. In some embodiments, the methods provided herein comprise administering to the individual a kinase inhibitor, e.g., in combination with another anti-cancer therapy. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR3, FGFR4, CSF1R, c-Met, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent. In some embodiments, the methods provided herein comprise administering to the individual an anti-angiogenic agent, e.g., in combination with another anti-cancer therapy. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v P3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy. In some embodiments, the methods provided herein comprise administering to the individual an anti-DNA repair therapy, e.g., in combination with another anti-cancer therapy. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer. In some embodiments, the methods provided herein comprise administering to the individual a radiosensitizer, e.g., in combination with another anti-cancer therapy. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent. In some embodiments, the methods provided herein comprise administering to the individual an anti-inflammatory agent, e.g., in combination with another anti-cancer therapy. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-xB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some embodiments, the anti-cancer therapy comprises an anti-hormonal agent. In some embodiments, the methods provided herein comprise administering to the individual an anti-hormonal agent, e.g., in combination with another anti-cancer therapy. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent. In some embodiments, the methods provided herein comprise administering to the individual an antimetabolite chemotherapeutic agent, e.g., in combination with another anti-cancer therapy. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOMED), azocytosine, deoxycytosine, pyridmidene, fludarabine (FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent. In some embodiments, the methods provided herein comprise administering to the individual a platinum-based chemotherapeutic agent, e.g., in combination with another anti-cancer therapy. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some aspects, provided herein are therapeutic formulations comprising an anti-cancer therapy provided herein, and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A formulation provided herein may also contain more than one active compound, for example, those with complementary activities that do not adversely affect each other. The type and effective amounts of such medicaments depend, for example, on the amount and type of active compound(s) present in the formulation, and clinical parameters of the subjects.

For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 1 19, Marcel Dekker, 2002.

Formulations to be used for in vivo administration are sterile. This is readily accomplished by filtration through sterile filtration membranes or other methods known in the art.

In some embodiments, the anti-cancer therapy is administered as a monotherapy. In some embodiments, the anti-cancer therapy is administered in combination with one or more additional anti-cancer therapies or treatments. In some embodiments, the one or more additional anti-cancer therapies or treatments include one or more anti-cancer therapies described herein. In some embodiments, the methods of the present disclosure comprise administration of any combination of any of the anti-cancer therapies provided herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy may be administered in conjunction with a radiation therapy. In some embodiments, the anti-cancer therapy for use in any of the methods described herein (e.g., as monotherapy or in combination with another therapy or treatment) is an anti-cancer therapy or treatment described by Pietrantonio et al., J Natl Cancer Inst (2017) 109(12) and/or by Wang et al., Cancers (2020) 12(2):426, which are hereby incorporated by reference.

### IV. ARTICLES OF MANUFACTURE OR KITS

Provided herein are kits or articles of manufacture comprising one or more oligonucleotides for detecting one or more mutations in a CD274 gene.

Further provided herein are kits or articles of manufacture comprising an anti-cancer therapy and a package insert comprising instructions for using the anti-cancer therapy in a method of treating or delaying progression of cancer, e.g., by administration to an individual from whom a sample comprising one or more mutations in a CD274 gene has been obtained.

In some embodiments, a kit provided herein comprises a reagent (e.g., one or more oligonucleotides, primers, probes or baits of the present disclosure) for detecting one or more mutations in a CD274 gene provided herein. In some embodiments, the kit comprises a reagent (e.g., one or more oligonucleotides, primers, probes or baits of the present disclosure) for detecting a wild-type counterpart of a CD274 gene. In some embodiments, the reagent comprises one or more oligonucleotides, primers, probes or baits of the present disclosure capable of hybridizing to a nucleic acid molecule that comprises or encodes a CD274 gene or a portion thereof comprising one or more mutations described herein, or to a wild-type counterpart of a CD274 gene. In some embodiments, the reagent comprises one or more oligonucleotides, primers, probes or baits of the present disclosure capable of distinguishing a nucleic acid molecule that comprises or encodes a CD274 gene or a portion thereof comprising one or more mutations described herein from a wild-type counterpart of the CD274 gene. In some embodiments, the kit is for use according to any method of detecting one or more mutations in a CD274 gene known in the art or described herein, such as sequencing, PCR, in situ hybridization methods, a nucleic acid hybridization assay, an amplification-based assay, a PCR-RFLP assay, real-time PCR, sequencing, next-generation sequencing, a screening analysis, FISH, spectral karyotyping, MFISH, comparative genomic hybridization, in situ hybridization, sequence-specific priming (SSP) PCR, HPLC, and mass-spectrometric genotyping. In some embodiments, a kit provided herein further comprises instructions for detecting one or more mutations in a CD274 gene, or a nucleic acid molecule that comprises or encodes a CD274 gene or a portion thereof comprising one or more mutations described herein, e.g., using one or more oligonucleotides, primers, probes or baits of the present disclosure.

Also provided herein are kits for detecting a PD-L1 polypeptide encoded by a CD274 gene, e.g., comprising one or more mutations described herein, or a fragment thereof. In some embodiments, a kit provided herein comprises a reagent (e.g., one or more antibodies of the present disclosure) for detecting a PD-L1 polypeptide encoded by a CD274 gene comprising one or more mutations described herein, or a fragment thereof. In some embodiments, the kit comprises a reagent (e.g., one or more antibodies of the present disclosure) for detecting the wild-type counterparts of a PD-L1 polypeptide provided herein. In some embodiments, the reagent comprises one or more antibodies of the present disclosure capable of binding to a PD-L1 polypeptide encoded by a CD274 gene comprising one or more mutations described herein, or a fragment thereof, or to wild-type counterparts of the PD-L1 polypeptide provided herein. In some embodiments, the reagent comprises one or more antibodies of the present disclosure capable of distinguishing a PD-L1 polypeptide encoded by a CD274 gene comprising one or more mutations described herein, or a fragment thereof, from wild-type counterparts of a PD-L1 polypeptide provided herein. In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography). In some embodiments, the kit further comprises instructions for detecting a PD-L1 polypeptide encoded by a CD274 gene comprising one or more mutations described herein, or a fragment thereof, e.g., using one or more antibodies of the present disclosure.

The article of manufacture may include, for example, a container and a label or package insert on or associated with the container. Suitable containers include, for example, bottles, vials, syringes, and the like. The container may be formed from a variety of materials such as glass or plastic. The container holds or contains a composition comprising the cancer medicament as the active agent and may have a sterile access port (e.g., the container may be an intravenous solution bag or a vial having a stopper pierceable by a hypodermic injection needle).

The article of manufacture may further include a second container comprising a pharmaceutically-acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

The article of manufacture of the present invention also includes information, for example in the form of a package insert, indicating that the composition is used for treating cancer, as described herein. The insert or label may take any form, such as paper or on electronic media such as a magnetically recorded medium (e.g., floppy disk), a CD-ROM, a Universal Serial Bus (USB) flash drive, and the like. The label or insert may also include other information concerning the pharmaceutical compositions and dosage forms in the kit or article of manufacture.

### V. EXPRESSION VECTORS, HOST CELLS AND RECOMBINANT CELLS

Provided herein are vectors comprising a nucleic acid molecule that comprises or encodes a CD274 gene or a portion thereof comprising one or more mutations described herein, a bait, a probe, or an oligonucleotide described herein, or fragments thereof.

In some embodiments, a vector provided herein comprises a nucleic acid molecule that comprises or encodes a CD274 gene or a portion thereof comprising one or more mutations described herein, or a nucleic acid molecule encoding a PD-L1 polypeptide encoded by a CD274 gene comprising one or more mutations described herein, or a fragment thereof.

In some embodiments, a vector provided herein is a nucleic acid molecule capable of transporting another nucleic acid to which it has been linked. In some embodiments, a vector is a plasmid, a cosmid or a viral vector. The vector may be capable of autonomous replication, or it can integrate into a host DNA. Viral vectors are also contemplated herein, including, e.g., replication defective retroviruses, adenoviruses and adeno-associated viruses.

In some embodiments, a vector provided herein comprises a nucleic acid molecule that comprises or encodes a CD274 gene or a portion thereof comprising one or more mutations described herein, a bait, a probe, or an oligonucleotide of the disclosure in a form suitable for expression thereof in a host cell. In some embodiments, the vector includes one or more regulatory sequences operatively linked to the nucleotide sequence to be expressed. In some embodiments, the one or more regulatory sequences include promoters (e.g., promoters derived from polyoma, Adenovirus 2, cytomegalovirus and Simian Virus 40), enhancers, and other expression control elements (e.g., polyadenylation signals). In some embodiments, a regulatory sequence directs constitutive expression of a nucleotide sequence (e.g., a nucleic acid molecule that comprises or encodes a CD274 gene or a portion thereof comprising one or more mutations described herein, baits, probes, or oligonucleotides described herein, or fragments thereof). In some embodiments, a regulatory sequence directs tissue-specific expression of a nucleotide sequence (e.g., a nucleic acid molecule that comprises or encodes a CD274 gene or a portion thereof comprising one or more mutations described herein, baits, probes, or oligonucleotides described herein, or fragments thereof). In some embodiments, a regulatory sequence directs inducible expression of a nucleotide sequence (e.g., a nucleic acid molecule that comprises or encodes a CD274 gene or a portion thereof comprising one or more mutations described herein, baits, probes, or oligonucleotides described herein, or fragments thereof). Examples of inducible regulatory sequences include, without limitation, promoters regulated by a steroid hormone, by a polypeptide hormone, or by a heterologous polypeptide, such as a tetracycline-inducible promoter. Examples of tissue- or cell-type-specific regulatory sequences include, without limitation, the albumin promoter, lymphoid-specific promoters, promoters of T cell receptors or immunoglobulins, neuron-specific promoters, pancreas-specific promoters, mammary gland-specific promoters, and developmentally-regulated promoters. In some embodiments, a vector provided herein comprises a nucleic acid molecule that comprises or encodes a CD274 gene or a portion thereof comprising one or more mutations described herein, a bait, a probe, or an oligonucleotide of the disclosure in the sense or the anti-sense orientation. In some embodiments, a vector (e.g., an expression vector) provided herein is introduced into host cells to thereby produce a polypeptide, e.g., a PD-L1 polypeptide encoded by a CD274 gene comprising one or more mutations described herein, or a fragment or mutant form thereof.

In some embodiments, the design of a vector provided herein depends on such factors as the choice of the host cell to be transformed, the level of expression desired, and the like. In some embodiments, expression vectors are designed for the expression of CD274 nucleic acid molecules, e.g., comprising one or more mutations described herein, baits, probes, or oligonucleotides described herein, or fragments thereof, in prokaryotic or eukaryotic cells, such as E. coli cells, insect cells (e.g., using baculovirus expression vectors), yeast cells, or mammalian cells. In some embodiments, a vector described herein is transcribed and translated in vitro, for example using T7 promoter regulatory sequences and T7 polymerase. In some embodiments, a vector (e.g., an expression vector) provided herein comprises a nucleic acid molecule that comprises or encodes a CD274 gene or a portion thereof comprising one or more mutations described herein, wherein the nucleotide sequence of the nucleic acid molecule described herein has been altered (e.g., codon optimized) so that the individual codons for each encoded amino acid are those preferentially utilized in the host cell.

Also provided herein are host cells, e.g., comprising a nucleic acid molecule that comprises or encodes a CD274 gene or a portion thereof comprising one or more mutations described herein, a PD-L1 polypeptide, or a portion thereof, encoded by a CD274 gene comprising one or more mutations described herein, baits, probes, vectors, or oligonucleotides of the disclosure. In some embodiments, a host cell (e.g., a recombinant host cell or recombinant cell) comprises a vector described herein (e.g., an expression vector described herein). In some embodiments, a nucleic acid molecule that comprises or encodes a CD274 gene or a portion thereof comprising one or more mutations described herein, bait, probe, vector, or oligonucleotide provided herein further includes sequences which allow it to integrate into the host cell's genome (e.g., through homologous recombination at a specific site). In some embodiments, a host cell provided herein is a prokaryotic or eukaryotic cell. Non limiting examples of host cells include, without limitation, bacterial cells (e.g., E. coli), insect cells, yeast cells, or mammalian cells (e.g., human cells, rodent cells, mouse cells, rabbit cells, pig cells, Chinese hamster ovary cells (CHO), or COS cells, e.g., COS-7 cells, CV-1 origin SV40 cells). A host cell described herein includes the particular host cell, as well as the progeny of such a cell. Because certain modifications may occur in succeeding generations due to either mutation or environmental influences, such progeny may not, in fact, be identical to the parent host cell.

A nucleic acid molecule that comprises or encodes a CD274 gene or a portion thereof comprising one or more mutations described herein, baits, probes, vectors, or oligonucleotides of the disclosure may be introduced into host cells using any suitable method known in the art, such as conventional transformation or transfection techniques (e.g., using calcium phosphate or calcium chloride co-precipitation, DEAE-dextran-mediated transfection, lipofection, or electroporation).

Also provided herein are methods of producing a PD-L1 polypeptide, or a portion thereof, encoded by a CD274 gene comprising one or more mutations described herein, e.g., by culturing a host cell described herein (e.g., into which a recombinant expression vector encoding a polypeptide has been introduced) in a suitable medium such that the PD-L1 polypeptide is produced. In another embodiment, the method further includes isolating the PD-L1 polypeptide from the medium or the host cell.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### VI. EXEMPLARY EMBODIMENTS

The following exemplary embodiments are representative of some aspects of the invention:

Exemplary Embodiment 1: A method of identifying an individual having cancer who may benefit from a treatment comprising an anti-cancer therapy, the method comprising detecting one or more mutations in a CD274 gene in a sample from the individual, wherein the presence of the one or more mutations in the CD274 gene in the sample identifies the individual as one who may or may not benefit from the anti-cancer therapy.

Exemplary Embodiment 2: A method of detecting the presence or absence of a cancer in an individual, the method comprising:
(a) detecting the presence or absence of a cancer in a sample from the individual; and
(b) detecting the presence or absence of one or more mutations in a CD274 gene in the sample.

Exemplary Embodiment 3: A method of selecting a therapy for an individual having cancer, the method comprising detecting one or more mutations in a CD274 gene in a sample from the individual, wherein the presence of the one or more mutations in the CD274 gene in the sample identifies the individual as one who may benefit from a treatment comprising an anti-cancer therapy.

Exemplary Embodiment 4: The method of any one of embodiments 1-3, wherein the presence of the one or more mutations in the CD274 gene in the sample identifies the individual as one who may have a cancer that is resistant to one or more immune checkpoint inhibitors.

Exemplary Embodiment 5: A method of identifying one or more treatment options for an individual having cancer, the method comprising:
(a) detecting one or more mutations in a CD274 gene in a sample from the individual; and
(b) generating a report comprising one or more treatment options identified for the individual based at least in part on the presence of the one or more mutations in the CD274 gene in the sample, wherein the one or more treatment options comprise an anti-cancer therapy.

Exemplary Embodiment 6: A method of identifying one or more treatment options for an individual having cancer, the method comprising:
(a) acquiring knowledge of one or more mutations in a CD274 gene in a sample from the individual; and
(b) generating a report comprising one or more treatment options identified for the individual based at least in part on said knowledge, wherein the one or more treatment options comprise an anti-cancer therapy.

Exemplary Embodiment 7: The method of embodiment 5 or embodiment 6, wherein the report identifies the individual as one who may have a cancer that is resistant to one or more immune checkpoint inhibitors.

Exemplary Embodiment 8: A method of selecting or not selecting a treatment for an individual having cancer, comprising acquiring knowledge of one or more mutations in a CD274 gene in a sample from an individual having cancer, wherein responsive to the acquisition of said knowledge: (i) the individual is classified as a candidate to receive treatment with an anti-cancer therapy, or the individual is not classified as a candidate to receive treatment with an anti-cancer therapy; and/or (ii) the individual is identified as likely to respond to a treatment that comprises an anti-cancer therapy, or the individual is identified as unlikely to respond to a treatment that comprises an anti-cancer therapy.

Exemplary Embodiment 9: The method of embodiment 8, wherein responsive to the acquisition of said knowledge: (i) the individual is classified as having a cancer that is resistant to one or more immune checkpoint inhibitors; and/or (ii) the individual is identified as unlikely to respond to a treatment that comprises one or more immune checkpoint inhibitors.

Exemplary Embodiment 10: A method of predicting survival of an individual having cancer, comprising acquiring knowledge of one or more mutations in a CD274 gene in a sample from the individual, wherein responsive to the acquisition of said knowledge, the individual is predicted to have shorter survival when treated with one or more immune checkpoint inhibitors, as compared to survival of an individual whose cancer does not comprise the one or more mutations in a CD274 gene.

Exemplary Embodiment 11: A method of predicting survival of an individual having a cancer treated with one or more immune checkpoint inhibitors, the method comprising acquiring knowledge of one or more mutations in a CD274 gene in a sample from the individual, wherein responsive to the acquisition of said knowledge, the individual is predicted to have shorter survival after treatment with the one or more immune checkpoint inhibitors, as compared to an individual whose cancer does not exhibit one or more mutations in a CD274 gene.

Exemplary Embodiment 12: A method of treating or delaying progression of cancer, comprising:
(a) acquiring knowledge of one or more mutations in a CD274 gene in a sample from an individual; and
(b) responsive to said knowledge, administering to the individual an effective amount of a treatment that comprises an anti-cancer therapy.

Exemplary Embodiment 13: A method of treating or delaying progression of cancer, comprising, responsive to acquiring knowledge of one or more mutations in a CD274 gene in a sample from an individual, administering to the individual an effective amount of a treatment that comprises an anti-cancer therapy.

Exemplary Embodiment 14: A method of monitoring an individual having cancer, comprising acquiring knowledge of one or more mutations in a CD274 gene in a sample from the individual, wherein responsive to the acquisition of said knowledge, the individual is predicted to have increased risk of cancer resistance to one or more immune checkpoint inhibitors, as compared to an individual whose cancer does not comprise one or more mutations in a CD274 gene.

Exemplary Embodiment 15: A method of evaluating an individual having cancer, comprising acquiring knowledge of one or more mutations in a CD274 gene in a sample from the individual, wherein responsive to the acquisition of said knowledge, the individual is predicted to have increased risk of cancer that is resistant to one or more immune checkpoint inhibitors, as compared to an individual whose cancer does not comprise the one or more mutations in a CD274 gene.

Exemplary Embodiment 16: A method of screening an individual having cancer, comprising acquiring knowledge of one or more mutations in a CD274 gene in a sample from the individual, wherein responsive to the acquisition of said knowledge, the individual is predicted to have increased risk of cancer that is resistant to one or more immune checkpoint inhibitors, as compared to an individual whose cancer does not comprise the one or more mutations in a CD274 gene.

Exemplary Embodiment 17: A method of treating or delaying progression of cancer, comprising:
(a) detecting one or more mutations in a CD274 gene in a sample from an individual; and
(b) administering to the individual an effective amount of a treatment that comprises an anti-cancer therapy.

Exemplary Embodiment 18: A method of diagnosing/assessing one or more mutations in a CD274 gene in a cancer in an individual, the method comprising:
(a) detecting one or more mutations in a CD274 gene in a sample from the individual; and
(b) providing an assessment of the one or more mutations in a CD274 gene.

Exemplary Embodiment 19: A method of diagnosing an immune checkpoint inhibitor-resistant cancer in an individual, the method comprising:
(a) detecting one or more mutations in a CD274 gene in a sample from the individual; and
(b) providing a diagnosis of an immune checkpoint inhibitor-resistant cancer in the individual.

Exemplary Embodiment 20: A method of detecting one or more mutations in a CD274 gene, the method comprising detecting the one or more mutations in a CD274 gene in a sample from an individual having a cancer.

Exemplary Embodiment 21: A method of detecting one or more mutations in a CD274 gene, the method comprising:
(a) providing a plurality of nucleic acids obtained from a sample from an individual, wherein the plurality of nucleic acids comprises nucleic acids encoding a CD274 gene;
(b) optionally, ligating one or more adaptors onto one or more nucleic acids from the plurality of nucleic acids;
(c) optionally, amplifying nucleic acids from the plurality of nucleic acids;
(d) optionally, capturing a plurality of nucleic acids corresponding to the CD274 gene;
(e) sequencing, by a sequencer, the plurality of nucleic acids to obtain a plurality of sequence reads corresponding to the CD274 gene;
(f) analyzing the plurality of sequence reads; and
(g) based on the analysis, detecting one or more mutations in a CD274 gene.

Exemplary Embodiment 22: The method of embodiment 21, wherein the plurality of nucleic acids corresponding to the CD274 gene is captured from the amplified nucleic acids by hybridization with a bait molecule.

Exemplary Embodiment 23: A method of detecting one or more mutations in a CD274 gene, the method comprising:
(a) providing a sample from an individual having a cancer, wherein the sample comprises one or more nucleic acids;
(b) preparing a nucleic acid sequencing library from the one or more nucleic acids in the sample;
(c) amplifying said library using a polymerase chain reaction (PCR);
(d) selectively enriching for one or more nucleic acids comprising CD274 nucleotide sequences in said library to produce an enriched sample;
(e) sequencing the enriched sample, thereby producing a plurality of sequencing reads;
(f) analyzing the plurality of sequencing reads for the presence of one or more mutations in a CD274 gene;
(g) detecting, based on the analyzing step, one or more mutations in a CD274 gene in the sample from the individual.

Exemplary Embodiment 24: A method of treating or delaying progression of cancer, comprising administering to an individual having cancer an effective amount of an anti-cancer therapy, wherein the cancer comprises one or more mutations in a CD274 gene.

Exemplary Embodiment 25: The method of any one of embodiments 1, 3-9, 12-13, 17, and 24, wherein the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

Exemplary Embodiment 26: The method of embodiment 25, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy.

Exemplary Embodiment 27: The method of embodiment 25, wherein the nucleic acid comprises a double-stranded RNA (dsRNA), a small interfering RNA (siRNA), or a small hairpin RNA (shRNA).

Exemplary Embodiment 28: The method of any one of embodiments 1-27, wherein the one or more mutations in a CD274 gene comprise one or more of a missense mutation, a truncation, a nonsense mutation, a splice site mutation, an insertion/deletion, and any combination thereof.

Exemplary Embodiment 29: The method of any one of embodiments 1-28, wherein the one or more mutations in a CD274 gene comprise two or more missense mutations.

Exemplary Embodiment 30: The method of any one of embodiments 1-29, wherein the one or more mutations in a CD274 gene comprise one or more missense mutations and a truncation.

Exemplary Embodiment 31: The method of any one of embodiments 1-30, wherein the one or more mutations in a CD274 gene comprise one or more mutations listed in Tables 1-4 and 6.

Exemplary Embodiment 32: The method of any one of embodiments 28-31, wherein the one or more mutations in a CD274 gene further comprise a CD274 gene deletion, wherein the CD274 gene is a deletion of a portion of the CD274 gene.

Exemplary Embodiment 33: The method of any one of embodiments 28-32, wherein the one or more mutations in a CD274 gene further comprise a CD274 genomic rearrangement or a CD274 gene fusion.

Exemplary Embodiment 34: The method of any one of embodiments 1-33, wherein the one or more mutations in a CD274 gene are somatic mutations or germline mutations.

Exemplary Embodiment 35: The method of any one of embodiments 1-34, wherein the one or more mutations in a CD274 gene are clonal mutations.

Exemplary Embodiment 36: The method of any one of embodiments 1-34, wherein the one or more mutations in a CD274 gene are sub-clonal mutations.

Exemplary Embodiment 37: The method of any one of embodiments 28-36, wherein the one or more mutations in a CD274 gene further comprise a CD274 gene amplification.

Exemplary Embodiment 38: The method of any one of embodiments 1-37, wherein the one or more mutations in a CD274 gene result in:
(a) low expression of a PD-L1 protein in the cancer,
(b) no expression of a PD-L1 protein in the cancer, or
(c) high expression of a PD-L1 protein in the cancer.

Exemplary Embodiment 39: The method of embodiment 38, wherein PD-L1 protein expression is assessed using an immunohistochemistry assay in sample obtained from the individual.

Exemplary Embodiment 40: The method of embodiment 38 or embodiment 39, wherein PD-L1 protein expression is assessed in tumor cells.

Exemplary Embodiment 41: The method of any one of embodiments 39-40, wherein a low expression of a PD-L1 protein in the cancer is assessed based on a tumor proportion score (TPS) of between 1% and 49%.

Exemplary Embodiment 42: The method of any one of embodiments 38-41, wherein the one or more mutations in a CD274 gene comprise one or more mutations listed in Table 2.

Exemplary Embodiment 43: The method of any one of embodiments 39-40, wherein no expression of a PD-L1 protein in the cancer is assessed based on a TPS of less than 1%.

Exemplary Embodiment 44: The method of any one of embodiments 38-40 and 43, wherein the one or more mutations in a CD274 gene comprise one or more mutations listed in Table 3.

Exemplary Embodiment 45: The method of any one of embodiments 39-40, wherein high expression of a PD-L1 protein in the cancer is assessed based on a TPS of 50% or greater.

Exemplary Embodiment 46: The method of any one of embodiments 38-40 and 45, wherein the one or more mutations in a CD274 gene comprise one or more mutations listed in Table 4.

Exemplary Embodiment 47: The method of any one of embodiments 38-46, wherein the one or more mutations in a CD274 gene comprise one or more missense mutations, optionally wherein the one or more mutations are clonal or sub-clonal mutations.

Exemplary Embodiment 48: The method of any one of embodiments 38-46, wherein the one or more mutations in a CD274 gene comprise a truncating mutation, optionally wherein the truncating mutation is a clonal or sub-clonal mutation.

Exemplary Embodiment 49: The method of any one of embodiments 1-48, wherein: (a) the one or more mutations in a CD274 gene reduce the interaction between a PD-L1 polypeptide encoded by the CD274 gene and a PD-1 receptor; and/or (b) the one or more mutations in a CD274 gene reduce the activity of a PD-L1 polypeptide encoded by the CD274 gene.

Exemplary Embodiment 50: The method of any one of embodiments 1-49, wherein the one or more mutations in a CD274 gene result in an immune checkpoint inhibitor resistant cancer.

Exemplary Embodiment 51: The method of any one of embodiments 1, 3-9, 12-13, 17, and 24-50, wherein the anti-cancer therapy is a therapy other than an immune checkpoint inhibitor.

Exemplary Embodiment 52: The method of embodiment 51, wherein the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

Exemplary Embodiment 53: The method of embodiment 52, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy.

Exemplary Embodiment 54: The method of embodiment 52, wherein the nucleic acid comprises a double-stranded RNA (dsRNA), a small interfering RNA (siRNA), or a small hairpin RNA (shRNA).

Exemplary Embodiment 55: The method of any one of embodiments 6-16 and 25-54, wherein the acquiring knowledge of one or more mutations in a CD274 gene comprises detecting the one or more mutations in a CD274 gene in the sample.

Exemplary Embodiment 56: The method of any one of embodiments 1-5, 7, 17-23, and 25-55, further comprising selectively enriching for one or more nucleic acids comprising nucleotide sequences that comprise the one or more mutations in the CD274 gene; wherein the selectively enriching produces an enriched sample.

Exemplary Embodiment 57: The method of any one of embodiments 1-5, 7, 17-23, and 25-56, wherein the one or more mutations in the CD274 gene are detected in the sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Exemplary Embodiment 58: The method of any one of embodiments 1-5, 7, 17-23, and 25-55, wherein the one or more mutations in the CD274 gene are detected in a PD-L1 polypeptide encoded by the CD274 gene.

Exemplary Embodiment 59: The method of embodiment 58, wherein the one or more mutations in the CD274 gene are detected in the sample by one or more of: immunoblotting, enzyme linked immunosorbent assay (ELISA), immunohistochemistry, or mass spectrometry.

Exemplary Embodiment 60: The method of any one of embodiments 1-20 and 23-59, wherein the cancer is a carcinoma, a sarcoma, a lymphoma, a leukemia, a myeloma, a germ cell cancer, or a blastoma.

Exemplary Embodiment 61: The method of any one of embodiments 1-20 and 23-60, wherein the cancer is a solid tumor.

Exemplary Embodiment 62: The method of any one of embodiments 1-20 and 23-60, wherein the cancer is a hematologic malignancy.

Exemplary Embodiment 63: The method of any one of embodiments 1-20 and 23-60, wherein the cancer is a cancer listed in Table 5 or Table 6.

Exemplary Embodiment 64: The method of any one of embodiments 1-20 and 23-63, wherein the cancer is diffuse large B-cell lymphoma, cutaneous squamous cell carcinoma, endometrial adenocarcinoma, unknown primary melanoma, or cutaneous melanoma.

Exemplary Embodiment 65: The method of any one of embodiments 1-20 and 23-60, wherein the cancer is a skin cancer.

Exemplary Embodiment 66: The method of embodiment 65, wherein the cancer comprises a tumor mutational burden (TMB) of ≥10 mutations/Megabase (mut/Mb).

Exemplary Embodiment 67: The method of embodiment 65, wherein the cancer comprises a TMB of less than 10 mut/Mb.

Exemplary Embodiment 68: The method of embodiment 66 or embodiment 67, wherein TMB is assessed based on about 0.79 megabases (Mb) of sequenced DNA.

Exemplary Embodiment 69: The method of embodiment 66 or embodiment 67, wherein TMB is assessed based on about 0.80 Mb of sequenced DNA.

Exemplary Embodiment 70: The method of embodiment 66 or embodiment 67, wherein TMB is assessed based on between about 0.83 Mb and about 1.14 Mb of sequenced DNA.

Exemplary Embodiment 71: The method of embodiment 66 or embodiment 67, wherein TMB is assessed based on about 1.1 Mb of sequenced DNA.

Exemplary Embodiment 72: The method of embodiment 66 or embodiment 67, wherein TMB is assessed based on up to about 1.24 Mb of sequenced DNA.

Exemplary Embodiment 73: The method of embodiment 66 or embodiment 67, wherein TMB is assessed based on up to about 1.1 Mb of sequenced DNA.

Exemplary Embodiment 74: The method of any one of embodiments 66 and 68-73, wherein the cancer comprises a TMB of at least about 100 mut/Mb, at least about 110 mut/Mb, at least about 120 mut/Mb, at least about 130 mut/Mb, at least about 140 mut/Mb, at least about 150 mut/Mb, or more.

Exemplary Embodiment 75: The method of any one of embodiments 66-74, wherein TMB is assessed by sequencing, whole exome sequencing, whole genome sequencing, gene-targeted sequencing, or next-generation sequencing.

Exemplary Embodiment 76: The method of any one of embodiments 65-75, wherein the cancer is cutaneous squamous cell carcinoma, cutaneous melanoma, or unknown primary melanoma.

Exemplary Embodiment 77: The method of any one of embodiments 1-20 and 23-60, wherein the cancer is a non-serous endometrial adenocarcinoma.

Exemplary Embodiment 78: The method of embodiment 77, wherein the cancer comprises a high microsatellite instability status (MSI).

Exemplary Embodiment 79: The method of embodiment 78, wherein MSI is assessed based on DNA sequencing of up to about 114 loci.

Exemplary Embodiment 80: The method of any one of embodiments 1-20 and 23-60, wherein the cancer is a cancer comprising a CD274 mutation as listed in Table 6.

Exemplary Embodiment 81: The method of any one of embodiments 1-20 and 23-80, wherein the cancer is metastatic.

Exemplary Embodiment 82: The method of any one of embodiments 1-20, 23, and 25-81, wherein the sample is obtained from the cancer.

Exemplary Embodiment 83: The method of any one of embodiments 1-23 and 25-82, wherein the sample is a formalin-fixed paraffin-embedded (FFPE) sample.

Exemplary Embodiment 84: The method of any one of embodiments 1-23 and 25-83, wherein the sample comprises fluid, cells, or tissue.

Exemplary Embodiment 85: The method of embodiment 84, wherein the sample comprises a tumor biopsy or a circulating tumor cell.

Exemplary Embodiment 86: The method of any one of embodiments 1-23 and 25-82, wherein the sample is a nucleic acid sample.

Exemplary Embodiment 87: The method of embodiment 86, wherein the nucleic acid sample comprises mRNA, genomic DNA, circulating tumor DNA, cell-free DNA, or cell-free RNA.

Exemplary Embodiment 88: The method of any one of embodiments 1-23 and 25-82, wherein the sample comprises one or more nucleic acids obtained from an FFPE sample from the individual.

Exemplary Embodiment 89: The method of embodiment 88, wherein the one or more nucleic acids comprise mRNA, genomic DNA, circulating tumor DNA, cell-free DNA, or cell-free RNA.

Exemplary Embodiment 90: The method of any one of embodiments 1-23 and 25-89, further comprising obtaining more than one sample from the individual at different time points.

Exemplary Embodiment 91: The method of embodiment 23 or embodiment 56, wherein the selectively enriching comprises: (a) combining a bait with the sample, thereby hybridizing the bait to the one or more nucleic acids in the sample and producing nucleic acid hybrids; and (b) isolating the nucleic acid hybrids to produce the enriched sample.

Exemplary Embodiment 92: The method of embodiment 91, wherein the bait comprises a capture nucleic acid molecule configured to hybridize to the one or more nucleic acids.

Exemplary Embodiment 93: The method of embodiment 92, wherein the capture nucleic acid molecule comprises between about 10 and about 30 nucleotides, between about 50 and about 1000 nucleotides, between about 100 and about 500 nucleotides, between about 100 and about 300 nucleotides, or between about 100 and 200 nucleotides.

Exemplary Embodiment 94: The method of any one of embodiments 91-93, wherein the bait is conjugated to an affinity reagent or to a detection reagent.

Exemplary Embodiment 95: The method of embodiment 94, wherein the affinity reagent is an antibody, an antibody fragment, or biotin, or wherein the detection reagent is a fluorescent marker.

Exemplary Embodiment 96: The method of any one of embodiments 92-95, wherein the capture nucleic acid molecule comprises a DNA, RNA, or mixed DNA/RNA molecule.

Exemplary Embodiment 97: The method of embodiment 23 or embodiment 56, wherein the selectively enriching comprises amplifying the one or more nucleic acids in the sample using a polymerase chain reaction (PCR) to produce the enriched sample.

Exemplary Embodiment 98: The method of any one of embodiments 91-97, further comprising sequencing the one or more nucleic acid molecules in the enriched sample.

Exemplary Embodiment 99: A kit comprising a probe or bait for detecting one or more mutations in a CD274 gene, optionally wherein the one or more mutations in a CD274 gene comprise one or more mutations listed in Tables 1-4 and 6.

Exemplary Embodiment 100: A nucleic acid encoding a CD274 gene comprising one or more mutations listed in Tables 1-4 and 6.

Exemplary Embodiment 101: A vector comprising the nucleic acid of embodiment 100.

Exemplary Embodiment 102: A host cell comprising the vector of embodiment 101.

Exemplary Embodiment 103: An antibody or antibody fragment that specifically binds to a PD-L1 polypeptide encoded by a CD274 gene comprising one or more mutations listed in Tables 1-4 and 6.

Exemplary Embodiment 104: A kit comprising the antibody or antibody fragment of embodiment 103.

Exemplary Embodiment 105: In vitro use of one or more oligonucleotides for detecting a CD274 gene, or a portion thereof, comprising one or more mutations listed in Tables 1-4 and 6.

Exemplary Embodiment 106: A kit comprising one or more oligonucleotides for detecting a CD274 gene, or a portion thereof, comprising one or more mutations listed in Tables 1-4 and 6.

Exemplary Embodiment 107: A system, comprising: a memory configured to store one or more program instructions; and one or more processors configured to execute the one or more program instructions, the one or more program instructions when executed by the one or more processors are configured to:
(a) obtain a plurality of sequence reads of one or more nucleic acids, wherein the one or more nucleic acids are derived from a sample obtained from an individual;
(b) analyze the plurality of sequence reads for the presence of one or more mutations in a CD274 gene; and
(c) detect, based on the analyzing, one or more mutations in a CD274 gene, in the sample.

Exemplary Embodiment 108: The system of embodiment 107, wherein the one or more mutations in a CD274 gene comprise one or more of a missense mutation, a truncation, a nonsense mutation, a splice site mutation, an insertion/deletion, and any combination thereof.

Exemplary Embodiment 109: The system of embodiment 107 or embodiment 108, wherein the one or more mutations in a CD274 gene comprise two or more missense mutations.

Exemplary Embodiment 110: The system of any one of embodiments 107-109, wherein the one or more mutations in a CD274 gene comprise one or more missense mutations and a truncation.

Exemplary Embodiment 111: The system of any one of embodiments 107-110, wherein the one or more mutations in a CD274 gene comprise one or more mutations listed in Tables 1-4 and 6.

Exemplary Embodiment 112: The system of any one of embodiments 108-111, wherein the one or more mutations in a CD274 gene further comprise a CD274 gene amplification.

Exemplary Embodiment 113: The system of any one of embodiments 108-112, wherein the one or more mutations in a CD274 gene further comprise a CD274 gene deletion, wherein the CD274 gene is a deletion of a portion of the CD274 gene.

Exemplary Embodiment 114: The system of any one of embodiments 108-113, wherein the one or more mutations in a CD274 gene further comprise a CD274 genomic rearrangement or a CD274 gene fusion.

Exemplary Embodiment 115: The system of any one of embodiments 107-114, wherein the one or more mutations in a CD274 gene are somatic mutations or germline mutations.

Exemplary Embodiment 116: The system of any one of embodiments 107-115, wherein the one or more mutations in a CD274 gene are clonal mutations.

Exemplary Embodiment 117: The system of any one of embodiments 107-116, wherein the one or more mutations in a CD274 gene are sub-clonal mutations.

Exemplary Embodiment 118: The system of any one of embodiments 107-117, wherein the plurality of sequence reads is obtained by sequencing, whole exome sequencing, whole genome sequencing, gene-targeted sequencing, or next-generation sequencing.

Exemplary Embodiment 119: A non-transitory computer readable storage medium comprising one or more programs executable by one or more computer processors for performing a method, comprising:
(a) obtaining, using the one or more processors, a plurality of sequence reads of one or more nucleic acids, wherein the one or more nucleic acids are derived from a sample obtained from an individual;
(b) analyzing, using the one or more processors, the plurality of sequence reads for the presence of one or more mutations in a CD274 gene; and
(c) detecting, using the one or more processors and based on the analyzing, one or more mutations in a CD274 gene, in the sample.

Exemplary Embodiment 120: The non-transitory computer readable storage medium of embodiment 119, wherein the one or more mutations in a CD274 gene comprise one or more of a missense mutation, a truncation, a nonsense mutation, a splice site mutation, an insertion/deletion, and any combination thereof.

Exemplary Embodiment 121: The non-transitory computer readable storage medium of embodiment 119 or embodiment 120, wherein the one or more mutations in a CD274 gene comprise two or more missense mutations.

Exemplary Embodiment 122: The non-transitory computer readable storage medium of any one of embodiments 119-120, wherein the one or more mutations in a CD274 gene comprise one or more missense mutations and a truncation.

Exemplary Embodiment 123: The non-transitory computer readable storage medium of any one of embodiments 119-122, wherein the one or more mutations in a CD274 gene comprise one or more mutations listed in Tables 1-4 and 6.

Exemplary Embodiment 124: The non-transitory computer readable storage medium of any one of embodiments 120-123, wherein the one or more mutations in a CD274 gene further comprise a CD274 gene amplification.

Exemplary Embodiment 125: The non-transitory computer readable storage medium of any one of embodiments 120-123, wherein the one or more mutations in a CD274 gene further comprise a CD274 gene deletion, wherein the CD274 gene is a deletion of a portion of the CD274 gene.

Exemplary Embodiment 126: The non-transitory computer readable storage medium of any one of embodiments 120-125, wherein the one or more mutations in a CD274 gene further comprise a CD274 genomic rearrangement or a CD274 gene fusion.

Exemplary Embodiment 127: The non-transitory computer readable storage medium of any one of embodiments 119-126, wherein the one or more mutations in a CD274 gene are somatic mutations or germline mutations.

Exemplary Embodiment 128: The non-transitory computer readable storage medium of any one of embodiments 119-127, wherein the one or more mutations in a CD274 gene are clonal mutations.

Exemplary Embodiment 129: The non-transitory computer readable storage medium of any one of embodiments 119-128, wherein the one or more mutations in a CD274 gene are sub-clonal mutations.

Exemplary Embodiment 130: The non-transitory computer readable storage medium of any one of embodiments 119-129, wherein the plurality of sequence reads is obtained by sequencing, whole exome sequencing, whole genome sequencing, gene-targeted sequencing, or next-generation sequencing.

Exemplary Embodiment 131: An anti-cancer therapy for use in a method of treating or delaying progression of cancer, wherein the method comprises administering the anti-cancer therapy to an individual, wherein one or more mutations in a CD274 gene are detected in a sample obtained from the individual.

Exemplary Embodiment 132: An anti-cancer therapy for use in the manufacture of a medicament for treating or delaying progression of cancer, wherein the medicament is to be administered to an individual, wherein one or more mutations in a CD274 gene are detected in a sample obtained from the individual.

### Examples

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this disclosure and scope of the appended claims.

### Example 1: Pan-Cancer Landscape of CD274 (PD-L1) Mutations and their Correlation with PD-L1 Protein Expression

The effects of non-amplification short variant (SV) “mutations” in CD274 (PD-L1) on its PD-L1 encoded protein expression and immune checkpoint inhibitor (ICPI) therapy are unknown.

This Example describes comprehensive genomic profiling (CGP) in a large pan-cancer genomic database that analyzed the landscape of CD274 (PD-L1) short variant mutations, as well as the correlation of identified mutations and PD-L1 protein expression.

### Materials and Methods

### Sample Cohort

Formalin-fixed, paraffin-embedded (FFPE) tissue of either whole section samples, biopsies, or cytology specimens were received as paraffin blocks or unstained slides from outside institutions during routine clinical care. A board-certified pathologist assigned a diagnosis for each specimen based on microscopic examination of a hematoxylin and eosin (H&E) stained slides from the FFPE tissue, the accompanying pathology report, and additional information provided by the ordering physician.

### Comprehensive Genomic Profiling

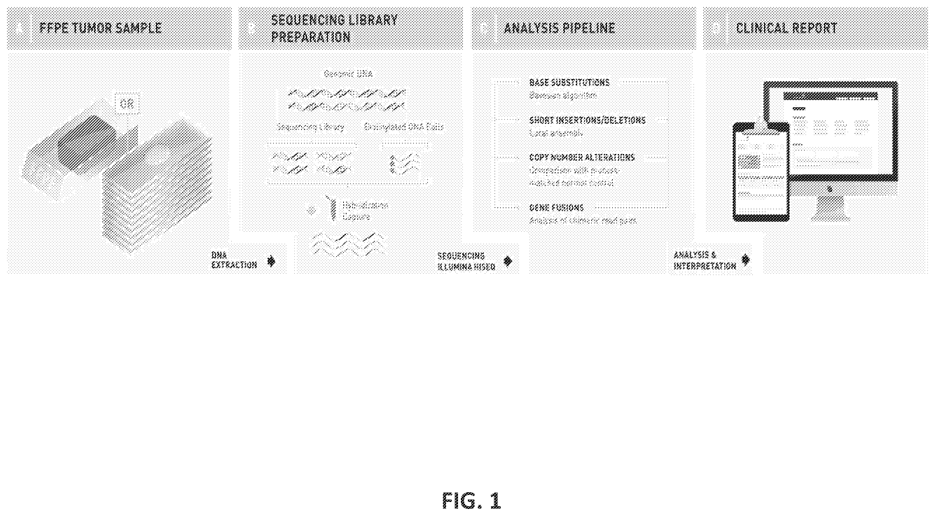

Comprehensive genomic profiling (CGP) was performed on hybridization-captured, adaptor ligation-based libraries using DNA and/or RNA extracted from FFPE tumor samples in a Clinical Laboratory Improvement Amendments (CLIA)-certified and College of American Pathologists (CAP)-accredited laboratory (see, FIG. 1). The samples were sequenced for up to 406 cancer related genes and select gene rearrangements (Frampton et al., Nat Biotechnol (2013) 31(11):1023-31). CD274 non-amplification short variant (SV) mutations were defined as missense mutations, truncations, splice site mutations, and insertion/deletions as previously described (Frampton et al., 2013). CD274 amplification was defined as ploidy+4. Tumor mutational burden (TMB) was determined on up to 1.24 megabases (Mb) of sequenced DNA, and TMB ≥10 mutations/Megabase (mut/Mb) was considered TMB-High (see, e.g., the website: www.fda.gov/drugs/drug-approvals-and-databases/fda-approves-pembrolizumab-adults-and-children-tmb-h-solid-tumors; and Chalmers et al., Genome Med (2017) 9(1):34). Microsatellite instability (MSI) analysis was performed from DNA sequencing up to 114 loci, and MSI-High (MSI-H) was considered positive, e.g., as described in www.fda.gov/drugs/resources-information-approved-drugs/fda-grants-accelerated-approval-pembrolizumab-first-tissuesite-agnostic-indication; and Trabucco et al., J Mol Diagn (2019) 21(6):1053-66. In addition, ultraviolet mutational signatures were called as described by Zehir et al., Nat Med (2017) 23(6):703-13.

### Clonality, Predicted Germline Versus Predicted Somatic Mutations, and Predicted Missense Functionality

A sub-clonal SV mutation was defined as a sample where <50% of tumor cells were predicted to harbor the variant based on both the variant allele fraction (VAF) and the pathologic and/or computational tumor cell purity estimates. A Somatic Germline Zygosity (SGZ) bioinformatics algorithm was used to determine whether mutations were likely somatic or germline, as previously described (see, Sun et al., PLOS Computational Biology (2018) 14(2):e1005965).

The functionality of missense CD274 mutations was assessed with several in silico methods including SIFT, MutationTaster, fathmm-MKL, and MetaSVM. The scores were re-calibrated to a rankscore so they could be compared to each other. See, e.g., Kim et al., BioData Min (2017) 10:2; Ng PC and Henikoff S, Nucleic Acids Res (2003) 31(13):3812-4; Shihab et al., Bioinformatics (2013) 29(12):1504-10; and Schwarz et al., Nat Methods (2014) 11(4):361-2. The rankscore was on a scale of 0 to 1, with 0 being predicted to be a non-functional protein and 1 being predicted to be a functional protein.

### DAKO PD-L1 IHC 22C3 Assay

For a subset of cases, the PD-L1 DAKO 22C3 assay was run according to manufacturer instructions in a CLIA-certified and CAP-accredited laboratory (www.accessdata.fda.gov/cdrh_docs/pdf15/P150013c.pdf). The IHC cases were interpreted by board-certified pathologists specifically trained on the DAKO tumor proportion scoring (TPS) method where tumor cell expression of PD-L1 was quantified. The DAKO TPS scoring method was defined as TPS=#PD-L1 positive tumor cells/(total # of PD-L1 positive+PD-L1 negative tumor cells) (see, www.agilent.com/cs/library/usermanuals/public/29158_pd-l1-ihc-22C3-pharmdx-nsclc-interpretation-manual.pdf). The PD-L1 22C3 TPS staining result was stratified into a negative (<1%), low expression (1-49%), or high expression (≥50%) category.

### Results

### Landscape of CD274 SV Mutations

Overall, the frequency of CD274 SV mutations was low (0.3%, 1,081/314,631) in the cohort of 314,631 samples. 577 unique variants were discovered, with some mutations recurrent and some mutations with only one occurrence in the entire cohort (see, Table 7). Of the 1,081 mutations, 49.9% (539/1081) were from metastatic specimens, and 42.3% (457/1081) were from primary specimens. In 7.8% of cases (85/1081) it was unknown whether they were from a primary or metastatic specimen. Of the 1,081 samples with CD274 SV mutations, only 1.4% (15/1081) had a co-occurring CD274 amplification.

TABLE 7
CD274 Variants.

| N samples | Genomic Alteration |
|---|---|
| 51 | R260H |
| 18 | R260C |
| 12 | R125Q |
| 11 | C272fs*13 |
| 10 | R86W, R113H |
| 9 | D215H |
| 8 | R140I, R140T, K271fs*44, R125* |
| 7 | T203del, H233Y, A18T, R86Q, E223K |
| 6 | Q77*, P24S, M266I, Y112C, S169N |
| 5 | A163V, G245E, E152*, E217Q, A232G, D284A |
| 4 | A85V, G177S, K280N, T290A, A52V, E158K, H220Y, K105Q, P235S, Q83H, S184F, |
| | R262I, E187Q, E217K, Q139R, E217*, K25N, M36I, Q173E, E205Q, D61N, F207L, |
| | G177D, K129N |
| 3 | D276Y, splice site 791 − 1G > A, G119D, N183S, P146L, P43S, R140K, A232T, A232V, |
| | E187D, E289D, G95E, M1I, P230S, Q66*, R265T, A109V, A5V, E39*, F9L, Q156H, |
| | R262K, R265K, R265fs*2, W13C, A232S, P216S, S279*, S79N, T148A, splice site 791 − |
| | 1G > T, D268N, G264E, M10I, P227S, R212K, E150K, G110R, K185N, L231V, P146S, |
| | S169C, splice site 52 + 2T > C, D145Y, E237K, E58K, L16P, L229P, N135S, A132V, G95R, |
| | I258M, I274M, R113C, S279L, V253I, E218K |
| 2 | A85fs*66, E188K, E223Q, E288Q, F67C, K75T, L241F, Q173H, Q47E, T179I, T201I, |
| | V193M, V21L, W13*, W57G, Y208C, D122N, D268H, D61Y, E188fs*7, E71Q, G119S, |
| | G70A, H78R, K178E, L27I, N131I, P235L, Q107K, S169R, T210S, V21A, V242I, A157T, |
| | D284N, E150*, E164Q, K46fs*3, L244M, L249V, Q66E, R2M, V165A, D268G, D26G, |
| | E288G, I8K, K62N, L142F, P133L, P133S, P216L, P43Q, T285I, V174G, V174I, V174L, |
| | V6I, A85T, C209Y, D103Y, D276N, E39K, E39Q, F257L, I8V, K75N, L106F, M59I, |
| | P133fs*2I, R198K, V143A, V147A, V30A, splice site 683 − 1G > A, D108N, D122Y, D73N, |
| | D90Y, G159C, G177C, H14Y, I38T, L190F, L53P, R213*, R238K, T221A, V29M, V68E, |
| | V76F, C272Y, D171G, E188V, E71K, H69N, K25R, L92H, T196A, T221I, V242A, A51V, |
| | A97V, A98T, E45K, E45Q, F257C, H69R, P161L, P216H, Q139E, Q91H, S80R, T181A, |
| | T256S, V23F, V68A, W57*, Y160H, splice site 394 + 1G > A |
| 1 | D90A, E152Q, E158V, E288K, E31G, F211fs*4, F4S, G245A, G245V, G252D, G264V, |
| | G70R, I126F, I126L, I199V, I38M, I3T, K162N, K189Q, L197P, L244V, L251fs*30, |
| | L50V, L94Q, N135D, N183D, N192I, P161S, P24A, Q275R, R113S, R238T, R265G, |
| | R82K, S283T, V111L, V253G, V269M, Y134*, splice site 630_682 + 272del325, P146*, |
| | P234S, Q173*, Q47R, R186G, R198T, R212I, S176G, T102I, T20A, T37K, V174A, |
| | V174D, W167*, Y28S, splice site 682 + 1G > A, splice site 790 + 1G > A, A121V, A222V, |
| | A246V, D215Y, D49G, D61V, D90fs*10, E158fs*15, E288D, F207fs*8, F211C, G252C, |
| | G264R, H240L, I166V, I65M, K25M, K89N, L16R, L190I, L255R, L53I, M59T, |
| | N183fs*22, N236D, P227A, P234R, Q100R, Q107*, Q156E, Q83R, R2K, S195I, T154I, |
| | T182P, T22S, V128L, V130L, V174F, Y12F, Y32C, Y56C, splice site 683 − 2A > C, C209F, |
| | C209S, D171N, D276E, I206M, I206T, I226L, I226M, I243V, K136E, L142fs*12, M115L, |
| | N200S, N204I, N219T, N236I, N96S, P227T, Q100H, Q282R, Q91E, R140*, R186*, |
| | R198G, R262G, R2W, T127fs*3, T203S, V143I, V44A, V55F, V68M, Y123H, splice site |
| | 53 − 49_82del79, *291Qext*42, A51D, A85S, A98V, C250G, C272R, C40fs*5, D145fs*8, |
| | D284Y, D49N, E152K, E158G, E188D, E188fs*12, E218Q, G119V, G264fs*2I, I64V, |
| | K124N, K46R, L248S, L287V, M10T, M10V, M36T, M36V, N131K, N138T, N17D, |
| | N192fs*13, N200D, N219I, N236H, S79G, T194A, T285S, W167C, W57S, Y118F, |
| | Y134C, splice site 790 + 1G > T, splice site 851 − 1G > C, *291Sext*42, A18S, A222G, A232P, |
| | A85fs*5, A97T, C114Y, C250Y, D215N, E150G, E31A, F211L, F7fs*27, G252S, G264W, |
| | H172Y, H240P, I258F, I64M, I64T, K281N, K41*, L190R, L249S, L251F, N131H, |
| | N138H, N138K, N35D, P43A, Q100*, Q66fs*13, S195R, T127A, T180I, T202I, T202R, |
| | T239S, T37A, V23I, V29G, splice site 683 − 1G > T, D145E, D61H, D73H, E164A, E187V, |
| | E237*, E237Q, E71*, F7L, G159R, H151fs*3, H240Y, I141V, N35K, N96H, N96Y, |
| | P230L, P230T, Q275*, R125L, R213del, S184P, S195N, T181I, T203A, T203I, T285A, |
| | T290_T290 > ?, T290fs*3+, V143F, V76I, splice site 52 + 1G > A, splice site 791 − 1G > C, |
| | *291Yext*42, A246D, D122E, D145H, E187K, E205K, E223V, E228V, E31K, F211I, |
| | F259L, G177V, G273D, H151L, H69L, I126S, I166L, I199fs*16, I243T, I54L, K189E, |
| | K271N, K280Q, L190V, L231M, L241*, L244P, L255M, L50M, L53R, L88F, M115T, |
| | M1?, M267I, N204K, N35H, N63Y, P234F, P43L, R186K, R213K, R2G, R82I, A157S, |
| | A51S, D103N, D26N, D90E, E158Q, E58G, F67I, H151R, H172Q, H220D, I3M, K162R, |
| | K185E, K263E, L106*, L142W, L261F, N183H, P234T, Q156K, R113L, R186T, R212T, |
| | R265I, S149Y, T154S, T285P, V165L, Y81H, splice site 394 + 2T > A, splice site |
| | 790 + 1_790 + 4delGTAG, A132D, A254G, A5D, D103H, D145N, D26Y, D276H, D61E, |
| | E188Q, E228G, E31_Y32insFTVTVPKDLYVVE, E60Q, F9fs*27, G110E, G33C, G70E, |
| | H14R, H172P, H69Y, H78Y, I101T, I141M, I258V, K162Q, K189M, K41_E45 > R, L16M, |
| | L197R, L255Q, L48S, M267T, N131S |

As shown in Table 7 and FIG. 2, the most common CD274 mutations were R260H (n=51), R260C (n=18), R125Q (n=12), C272fs*13 (n=11), R86W (n=10), and R113H (n=10). R260C/H was the most frequent recurrent missense mutation; both substitutions have been observed in the germline of healthy subjects (gnomAD; see, e.g., gnomad.broadinstitute.org).

A Somatic Germline Zygosity (SGZ) algorithm was used to determine whether the mutations were likely somatic or germline mutations. Based on the SGZ algorithm, 29.0% (20169) of the codon R260 mutations were likely somatic, 55.1% (38/69) were likely germline, and the algorithm could not predict whether the variant was germline or somatic in 16.0% (11/69) of the samples. In addition, when all the missense mutations (n=974) were examined, 51.0% (497/974) were predicted to be somatic, 24.7% (241/974) were predicted to be germline, and the algorithm could not predict whether the variant was germline or somatic in 24.2% (236/974) of the cases (Table 8).

TABLE 8
Predicted Germline or Somatic Missense Mutations with Somatic Germline Zygosity (SGZ) Algorithm.

| | | | | | | | | | |
|---|---|---|---|---|---|---|---|---|---|
| A109V; A | D215H; A | E218K; S | G245V; S | K129N; S | M266I; A | P43A; S | R212K; S | R86Q; G | V147A; A |
| A109V; A | D215H; A | E218K; S | G252C; A | K136E; A | M266I; G | P43L; S | R212T; A | R86Q; G | V147A; G |
| A109V; G | D215H; A | E218K; S | G252D; G | K162N; S | M266I; S | P43Q; S | R213K; A | R86Q; G | V165A; A |
| A121V; S | D215H; A | E218Q; S | G252S; G | K162Q; S | M266I; S | P43Q; S | R238K; A | R86Q; S | V165A; S |
| A132D; A | D215H; G | E223K; S | G264E; S | K162R; A | M266I; S | P43S; G | R238K; A | R86Q; S | V165L; S |
| A132V; A | D215H; S | E223K; S | G264E; S | K178E; G | M266I; S | P43S; S | R238T; S | R86Q; S | V174A; S |
| A132V; S | D215H; S | E223K; S | G264E; S | K178E; G | M266I; S | P43S; S | R260C; A | R86W; A | V174D; G |
| A132V; S | D215H; S | E223K; S | G264R; S | K185E; S | M267I; A | Q100H; S | R260C; A | R86W; A | V174F; A |
| A157S; S | D215H; S | E223K; S | G264V; S | K185N; A | M267T; A | Q100R; A | R260C; G | R86W; A | V174G; A |
| A157T; A | D215N; A | E223K; S | G264W; A | K185N; A | M36I; A | Q107K; G | R260C; G | R86W; G | V174G; S |
| A157T; S | D215Y; S | E223K; S | G273D; S | K185N; S | M36I; S | Q107K; S | R260C; G | R86W; G | V174I; A |
| A163V; A | D268G; A | E223Q; A | G33C; S | K189E; S | M36I; S | Q139E; G | R260C; G | R86W; G | V174I; G |
| A163V; S | D268G; S | E223Q; A | G70A; S | K189M; G | M36I; S | Q139E; S | R260C; G | R86W; G | V174L; G |
| A163V; S | D268H; G | E223V; G | G70A; S | K189Q; S | M36T; A | Q139R; A | R260C; G | R86W; G | V174L; G |
| A163V; S | D268H; G | E228G; G | G70E; S | K25M; A | M36V; G | Q139R; G | R260C; G | R86W; S | V193M; G |
| A163V; S | D268N; A | E228V; S | G70R; A | K25N; A | M59I; A | Q139R; G | R260C; G | R86W; S | V193M; S |
| A18S; G | D268N; S | E237K; A | G95E; S | K25N; S | M59I; S | Q139R; S | R260C; G | S149Y; S | V21A; A |
| A18T; A | D268N; S | E237K; S | G95E; S | K25N; S | M59T; A | Q156E; S | R260C; G | S169C; S | V21A; S |
| A18T; A | D26G; S | E237K; S | G95E; S | K25N; S | N131H; S | Q156H; A | R260C; S | S169C; S | V21L; S |
| A18T; S | D26G; S | E237Q; S | G95R; G | K25R; G | N131I; A | Q156H; G | R260C; S | S169C; S | V21L; S |
| A18T; S | D26N; S | E288D; G | G95R; G | K25R; G | N131I; G | Q156H; G | R260C; S | S169N; A | V23F; A |
| A18T; S | D26Y; S | E288G; A | G95R; S | K263E; S | N131K; S | Q156K; G | R260C; S | S169N; A | V23F; S |
| A18T; S | D276E; S | E288G; S | H14R; S | K271N; S | N131S; G | Q173E; A | R260C; S | S169N; G | V23I; A |
| A18T; S | D276H; S | E288G; S | H14Y; A | K280N; S | N135D; S | Q173E; G | R260C; S | S169N; G | V242A; A |
| A222G; S | D276N; S | E288K; S | H14Y; S | K280N; S | N135S; G | Q173E; S | R260H; A | S169N; S | V242A; S |
| A222V; S | D276N; S | E288Q; A | H151L; G | K280N; S | N135S; S | Q173E; S | R260H; A | S169N; S | V242I; G |
| A232G; A | D276Y; S | E288Q; S | H151R; G | K280N; S | N135S; S | Q173H; S | R260H; A | S169R; A | V242I; G |
| A232G; A | D276Y; S | E289D; A | H172P; A | K280Q; S | N138H; S | Q173H; S | R260H; A | S169R; S | V253G; S |
| A232G; G | D276Y; S | E289D; G | H172Q; A | K281N; A | N138K; S | Q275R; S | R260H; A | S176G; S | V253I; G |
| A232G; G | D284A; A | E289D; S | H172Y; S | K46R; S | N138T; S | Q282R; G | R260H; A | S184F; S | V253I; S |
| A232G; G | D284A; A | E31A; A | H220D; G | K62N; G | N17D; A | Q47E; G | R260H; A | S184F; S | V253I; S |
| A232P; A | D284A; A | E31G; G | H220Y; A | K62N; S | N183D; S | Q47E; G | R260H; A | S184F; S | V269M; S |
| A232S; G | D284A; G | E31K; S | H220Y; A | K75N; A | N183H; A | Q47R; S | R260H; A | S184F; S | V29G; A |
| A232S; S | D284A; G | E39K; S | H220Y; G | K75N; A | N183S; A | Q66E; S | R260H; G | S184P; A | V29M; G |
| A232S; S | D284N; S | E39K; S | H220Y; G | K75T; A | N183S; S | Q66E; S | R260H; G | S195I; G | V29M; S |
| A232T; A | D284N; S | E39Q; G | H233Y; A | K75T; G | N183S; S | Q83H; A | R260H; G | S195N; S | V30A; S |
| A232T; S | D284Y; A | E39Q; S | H233Y; G | K89N; A | N192I; G | Q83H; S | R260H; G | S195R; S | V30A; S |
| A232T; S | D49G; S | E45K; A | H233Y; G | L106F; S | N200D; S | Q83H; S | R260H; G | S279L; A | V44A; G |
| A232V; S | D49N; S | E45K; A | H233Y; G | L106F; S | N200S; A | Q83H; S | R260H; G | S279L; A | V55F; S |
| A232V; S | D61E; S | E45Q; S | H233Y; G | L142F; G | N204I; A | Q83R; S | R260H; G | S279L; S | V68A; G |
| A232V; S | D61H; A | E45Q; S | H233Y; G | L142F; S | N204K; G | Q91E; G | R260H; G | S283T; S | V68A; G |
| A246D; S | D61N; G | E58G; S | H233Y; G | L142W; G | N219I; A | Q91H; G | R260H; G | S79G; S | V68E; G |
| A246V; S | D61N; S | E58K; S | H240L; S | L16M; S | N219T; S | Q91H; G | R260H; G | S79N; G | V68E; G |
| A254G; S | D61N; S | E58K; S | H240P; A | L16P; G | N236D; A | R113C; A | R260H; G | S79N; G | V68M; S |
| A51D; A | D61N; S | E58K; S | H240Y; S | L16P; G | N236H; S | R113C; A | R260H; G | S79N; G | V6I; A |
| A51S; S | D61V; S | E60Q; S | H69L; S | L16P; G | N236I; S | R113C; A | R260H; G | S80R; A | V6I; G |
| A51V; A | D61Y; S | E71K; G | H69N; S | L16R; A | N35D; S | R113H; A | R260H; G | S80R; G | V76F; G |
| A51V; S | D61Y; S | E71K; S | H69N; S | L190F; A | N35H; S | R113H; A | R260H; G | T102I; A | V76F; G |
| A52V; A | D73H; S | E71Q; A | H69R; G | L190F; S | N35K; S | R113H; A | R260H; G | T127A; S | V76I; S |
| A52V; A | D73N; A | E71Q; S | H69R; S | L190I; A | N63Y; S | R113H; G | R260H; G | T148A; A | W13C; G |
| A52V; S | D73N; S | F207L; A | H69Y; G | L190R; S | N96H; A | R113H; S | R260H; G | T148A; A | W13C; S |
| A52V; S | D90A; S | F207L; S | H78R; A | L190V; S | N96S; S | R113H; S | R260H; G | T148A; G | W13C; S |
| A5D; S | D90E; S | F207L; S | H78R; S | L197P; A | N96Y; A | R113H; S | R260H; G | T154I; G | W167C; S |
| A5V; G | D90Y; G | F207L; S | H78Y; G | L197R; S | P133L; G | R113H; S | R260H; G | T154S; A | W57G; A |
| A5V; S | D90Y; G | F211C; S | I101T; S | L229P; G | P133L; S | R113H; S | R260H; G | T179I; G | W57G; G |
| A5V; S | E150G; S | F211I; G | I126F; S | L229P; S | P133S; G | R113H; S | R260H; G | T179I; S | W57S; S |
| A85S; A | E150K; S | F211L; G | I126F; S | L229P; S | P133S; S | R113L; S | R260H; G | T180I; S | Y112C; S |
| A85T; G | E150K; S | F257C; G | I126L; G | L231M; S | P146L; S | R113S; A | R260H; G | T181A; S | Y112C; S |
| A85T; S | E150K; S | F257C; G | I126S; S | L231V; S | P146L; S | R125L; A | R260H; G | T181A; S | Y112C; S |
| A85T; S | E152K; A | F257L; A | I141M; A | L231V; S | P146L; S | R125Q; A | R260H; G | T181I; S | Y112C; S |
| A85V; A | E152Q; S | F257L; A | I141V; A | L231V; S | P146S; A | R125Q; A | R260H; G | T182P; G | Y112C; S |
| A85V; A | E158G; A | F259L; G | I166L; S | L241F; A | P146S; S | R125Q; A | R260H; G | T194A; A | Y112C; S |
| A85V; S | E158K; A | F4S; S | I166V; S | L241F; G | P146S; S | R125Q; G | R260H; S | T196A; G | Y118F; A |
| A85V; S | E158K; A | F67C; A | I199V; S | L244M; A | P161L; S | R125Q; S | R260H; S | T196A; G | Y123H; S |
| A97T; A | E158K; S | F67C; S | I206M; S | L244M; S | P161L; S | R125Q; S | R260H; S | T201I; A | Y12F; G |
| A97V; S | E158K; S | F67I; S | I206T; A | L244P; G | P161S; S | R125Q; S | R260H; S | T201I; S | Y134C; S |
| A97V; S | E158Q; G | F7L; A | I226L; A | L244V; S | P216H; S | R125Q; S | R260H; S | T202I; A | Y160H; A |
| A98T; G | E158V; G | F9L; A | I226M; S | L248S; G | P216H; S | R125Q; S | R260H; S | T202R; S | Y160H; G |
| A98T; S | E164A; S | F9L; S | I243T; A | L249S; A | P216L; S | R125Q; S | R260H; S | T203A; G | Y208C; G |
| A98V; S | E164Q; S | F9L; S | I243V; G | L249V; G | P216L; S | R125Q; S | R260H; S | T203I; G | Y208C; S |
| C114Y; S | E164Q; S | G110E; A | I258F; S | L249V; S | P216S; G | R125Q; S | R260H; S | T203S; G | Y28S; S |
| C209F; S | E187D; A | G110R; S | I258M; A | L251F; A | P216S; S | R140I; A | R260H; S | T20A; A | Y32C; S |
| C209S; A | E187D; S | G110R; S | I258M; A | L255M; G | P216S; S | R140I; G | R260H; S | T210S; G | Y56C; G |
| C209Y; S | E187D; S | G110R; S | I258M; S | L255Q; S | P227A; A | R140I; S | R260H; S | T210S; S | Y81H; S |
| C209Y; S | E187K; A | G119D; G | I258V; G | L255R; S | P227S; A | R140I; S | R260H; S | T221A; A | |
| C250G; G | E187Q; A | G119D; G | I274M; A | L261F; S | P227S; A | R140I; S | R260H; S | T221A; S | |
| C250Y; A | E187Q; A | G119D; S | I274M; G | L261F; S | P227S; S | R140I; S | R262G; G | T221I; G | |
| C272R; S | E187Q; G | G119S; S | I274M; G | L27I; G | P227T; S | R140I; S | R262I; A | T221I; S | |
| C272Y; A | E187Q; S | G119S; S | I38M; A | L27I; S | P230L; S | R140I; S | R262I; A | T22S; S | |
| C272Y; S | E187V; S | G119V; G | I38T; G | L287V; S | P230S; A | R140K; A | R262I; S | T239S; G | |
| D103H; S | E188D; S | G159C; S | I38T; S | L48S; G | P230S; A | R140K; S | R262I; S | T256S; A | |
| D103N; S | E188K; G | G159C; S | I3M; S | L50M; S | P230S; G | R140K; S | R262K; A | T256S; G | |
| D103Y; A | E188K; S | G159R; A | I3T; A | L50V; S | P230T; G | R140T; A | R262K; S | T285A; S | |
| D103Y; S | E188Q; A | G177C; A | I54L; S | L53I; S | P234F; S | R140T; A | R262K; S | T285I; G | |
| D108N; S | E188V; S | G177C; S | I64M; A | L53P; A | P234R; G | R140T; A | R265G; G | T285I; S | |
| D108N; S | E188V; S | G177D; S | I64T; G | L53P; S | P234S; S | R140T; G | R265I; S | T285P; A | |
| D122E; A | E205K; S | G177D; S | I64V; G | L53P; S | P234T; S | R140T; S | R265K; S | T285S; A | |
| D122N; S | E205Q; A | G177D; S | I65M; G | L53R; A | P235L; A | R140T; S | R265K; S | T290A; A | |
| D122N; S | E205Q; S | G177D; S | I8K; G | L88F; S | P235L; S | R140T; S | R265K; S | T290A; G | |
| D122Y; A | E205Q; S | G177S; A | I8K; G | L88F; S | P235S; A | R140T; S | R265T; G | T290A; G | |
| D122Y; S | E205Q; S | G177S; G | I8V; G | L92H; G | P235S; A | R186G; G | R265T; S | T290A; S | |
| D145E; S | E217K; A | G177S; S | I8V; G | L92H; S | P235S; S | R186K; S | R265T; S | T37A; S | |
| D145H; A | E217K; A | G177S; S | K105Q; A | L94Q; S | P235S; S | R186T; S | R2G; A | T37K; G | |
| D145N; A | E217K; G | G177V; S | K105Q; G | M10I; S | P24A; A | R198G; A | R2K; G | V111L; S | |
| D145Y; A | E217K; S | G245A; G | K105Q; G | M10I; S | P24S; A | R198K; S | R2M; A | V128L; S | |
| D145Y; G | E217Q; A | G245E; A | K105Q; G | M10I; S | P24S; A | R198K; S | R2M; S | V130L; G | |
| D145Y; G | E217Q; G | G245E; A | K124N; A | M10T; S | P24S; G | R198T; S | R2W; S | V143A; A | |
| D171G; A | E217Q; G | G245E; S | K129N; G | M10V; A | P24S; G | R212I; A | R82I; S | V143A; G | |
| D171G; A | E217Q; S | G245E; S | K129N; G | M115L; S | P24S; S | R212K; S | R82K; S | V143F; S | |
| D171N; G | E217Q; S | G245E; S | K129N; G | M115T; G | P24S; S | R212K; S | R86Q; G | V143I; A | |

Predicted Germline or Somatic Missense Mutations: G = predicted germline; S = predicted somatic; and A = unable to predict germline or somatic.

C272fs*13 is at an indel at a poly-A homopolymer, a sequence context that is highly mutable in the setting of MSI-H status. This mutation was significantly enriched in the MSI-H group (0.01%, 5/5139) when compared to the non-MSI-H group (0.002%, 6/309,492) (Fisher's Exact Test, p<0.0001), suggesting that the variant is often a result of mismatch repair protein deficiency. This finding is reflected in the high CD274 SV mutation frequency in non-serous endometrial adenocarcinomas in this study. Thus, MSI was likely a mechanism for development of CD274 mutations in non-serous endometrial adenocarcinoma.

The types of mutations in this cohort also varied, with missense mutations being the most common (83.8%, 906/1081) and insertion/deletions being less common (0.8%, 9/1081) (Table 9). Multiple samples had complex CD274 mutations, defined as more than one CD274 genomic alteration observed in the sample. The most common type of complex CD274 mutation was alterations with two missense mutations (1.9%, 21/1081), while other complex mutations included a CD274 mutation with concurrent CD274 amplification (1.4%, 15/1081) and/or rearrangement (0.3%, 3/1081).

TABLE 9
Types of CD274 Mutations Identified.

| Total | Prevalence | |
|---|---|---|
| (n = | by mutation | |
| 1081) | type | Type of CD274 mutations |

Simple mutations

| | | |
|---|---|---|
| 906 | 83.8% | Missense mutations |
| 97 | 9.0% | Truncations |
| 24 | 2.2% | Splice site mutations |
| 9 | 0.8% | Insertion/deletions |

Complex Mutations

| | | |
|---|---|---|
| 21 | 1.9% | Two missense mutations |
| 13 | 1.2% | Missense mutation with amplification |
| 6 | 0.6% | Missense mutation with truncation |
| 1 | 0.1% | S149Y, CD274 - PLGRKT fusion |
| 1 | 0.1% | R140T, E187Q, deletion, CD274 - CD274 |
| | | (intragenic rearrangement) |
| 1 | 0.1% | L106*, amplification (copy number 90), |
| | | CD274 - CD274 (intragenic rearrangement) |
| 1 | 0.1% | P235S, S279L, amplification (copy number 12) |
| 1 | 0.1% | R86Q, G95E, W57*, splice site 682 + 1G > A |

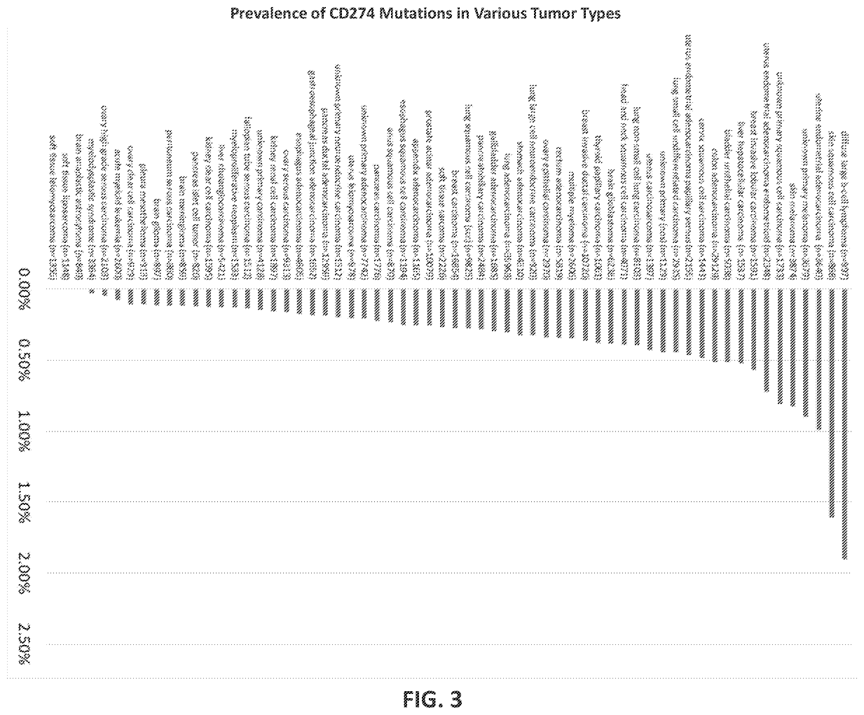

The prevalence of CD274 mutations also varied depending on tumor type. The top five tumor types (minimum 800 total samples) with the highest rates of CD274 mutations in descending order were: diffuse large B-cell lymphoma (1.9%, 19/997), cutaneous squamous cell carcinoma (1.6%, 14/868), endometrial adenocarcinoma (1.0%, 36/3740), unknown primary melanoma (0.9%, 33/3679), and cutaneous melanoma (0.8%, 32/3874) (see, FIG. 3 and Table 10).

TABLE 10
Frequency of CD274 Mutations for Each Diagnosis (only total n > 100 shown).

| Diagnosis | Frequency | N | Total |
|---|---|---|---|
| Lung adenocarcinoma | 0.31% | 110 | 35968 |
| Colon adenocarcinoma (CRC) | 0.51% | 150 | 29129 |
| Breast carcinoma (NOS) | 0.27% | 46 | 16854 |
| Pancreas ductal adenocarcinoma | 0.19% | 24 | 12959 |
| Breast invasive ductal carcinoma (IDC) | 0.36% | 39 | 10720 |
| Prostate acinar adenocarcinoma | 0.26% | 26 | 10079 |
| Lung squamous cell carcinoma (SCC) | 0.27% | 27 | 9825 |
| Ovary serous carcinoma | 0.16% | 15 | 9313 |
| Lung non-small cell lung carcinoma (NSCLC) (NOS) | 0.39% | 32 | 8103 |
| Unknown primary adenocarcinoma | 0.21% | 16 | 7742 |
| Brain glioblastoma (GBM) | 0.38% | 24 | 6238 |
| Rectum adenocarcinoma (CRC) | 0.34% | 20 | 5819 |
| Liver cholangiocarcinoma | 0.13% | 7 | 5421 |
| Bladder urothelial (transitional cell) carcinoma | 0.52% | 26 | 5038 |
| Esophagus adenocarcinoma | 0.17% | 8 | 4605 |
| Stomach adenocarcinoma (NOS) | 0.32% | 14 | 4310 |
| Head and neck squamous cell carcinoma (HNSCC) | 0.39% | 16 | 4071 |
| Unknown primary carcinoma (CUP) (NOS) | 0.15% | 6 | 4128 |
| Skin melanoma | 0.83% | 32 | 3874 |
| Unknown primary melanoma | 0.90% | 33 | 3679 |
| Uterus endometrial adenocarcinoma (NOS) | 0.99% | 36 | 3640 |
| Myelodysplastic syndrome (MDS) (NOS) | 0.03% | 1 | 3364 |
| Ovary epithelial carcinoma | 0.34% | 10 | 2970 |
| Lung small cell undifferentiated carcinoma | 0.44% | 13 | 2935 |
| Multiple myeloma | 0.35% | 9 | 2606 |
| Acute myeloid leukemia (AML) (NOS) | 0.08% | 2 | 2600 |
| Pancreatobiliary carcinoma | 0.28% | 7 | 2484 |
| Uterus endometrial adenocarcinoma endometrioid | 0.72% | 17 | 2348 |
| Soft tissue sarcoma (NOS) | 0.27% | 6 | 2226 |
| Uterus endometrial adenocarcinoma papillary serous | 0.46% | 10 | 2155 |
| Ovary high grade serous carcinoma | 0.05% | 1 | 2103 |
| Kidney renal cell carcinoma | 0.16% | 3 | 1897 |
| Pancreas carcinoma (NOS) | 0.23% | 4 | 1776 |
| Unknown primary squamous cell carcinoma (SCC) | 0.81% | 14 | 1733 |
| Gallbladder adenocarcinoma | 0.30% | 5 | 1685 |
| Gastroesophageal junction adenocarcinoma | 0.18% | 3 | 1652 |
| Breast invasive lobular carcinoma (ILC) | 0.57% | 9 | 1591 |
| Kidney clear cell carcinoma | 0.13% | 2 | 1595 |
| Unknown primary undifferentiated neuroendocrine carcinoma | 0.20% | 3 | 1512 |
| Liver hepatocellular carcinoma (HCC) | 0.52% | 8 | 1537 |
| Fallopian tube serous carcinoma | 0.13% | 2 | 1512 |
| Myeloproliferative neoplasm (MPN) (NOS) | 0.13% | 2 | 1533 |
| Cervix squamous cell carcinoma (SCC) | 0.49% | 7 | 1443 |
| Soft tissue leiomyosarcoma | 0.00% | 0 | 1355 |
| Uterus carcinosarcoma | 0.43% | 6 | 1397 |
| Appendix adenocarcinoma | 0.26% | 3 | 1165 |
| Unknown primary (NOS) | 0.44% | 5 | 1129 |
| Esophagus squamous cell carcinoma (SCC) | 0.25% | 3 | 1194 |
| Soft tissue liposarcoma | 0.00% | 0 | 1148 |
| Thyroid papillary carcinoma | 0.38% | 4 | 1061 |
| Pleura mesothelioma | 0.11% | 1 | 913 |
| Diffuse large b-cell lymphoma (DLBCL) (NOS) | 1.91% | 19 | 997 |
| Uterus leiomyosarcoma | 0.20% | 2 | 978 |
| Lung large cell neuroendocrine carcinoma | 0.33% | 3 | 920 |
| Brain glioma (NOS) | 0.11% | 1 | 897 |
| Ovary clear cell carcinoma | 0.11% | 1 | 929 |
| Brain anaplastic astrocytoma | 0.00% | 0 | 849 |
| Skin squamous cell carcinoma (SCC) | 1.61% | 14 | 868 |
| Anus squamous cell carcinoma (SCC) | 0.23% | 2 | 870 |
| Peritoneum serous carcinoma | 0.12% | 1 | 860 |
| Brain meningioma | 0.12% | 1 | 859 |
| Pancreas islet cell tumor | 0.12% | 1 | 820 |
| Brain astrocytoma | 0.40% | 3 | 747 |
| Cervix adenocarcinoma | 0.39% | 3 | 773 |
| Bile duct adenocarcinoma | 0.13% | 1 | 763 |
| Kidney urothelial carcinoma | 0.41% | 3 | 733 |
| Small intestine adenocarcinoma | 0.94% | 7 | 747 |
| Unknown primary urothelial carcinoma | 0.26% | 2 | 758 |
| Bone osteosarcoma | 0.00% | 0 | 703 |
| Duodenum adenocarcinoma | 0.74% | 5 | 679 |
| Acute b-lymphoblastic leukemia-lymphoma (B-ALL) | 0.00% | 0 | 714 |
| Chronic lymphocytic leukemia/small lymphocytic lymphoma (CLL/SLL) | 0.00% | 0 | 658 |
| Unknown primary gist | 0.00% | 0 | 628 |
| Ovary endometrioid adenocarcinoma | 0.00% | 0 | 594 |
| Acute lymphoblastic leukemia-lymphoma (ALL) (NOS) | 0.51% | 3 | 592 |
| Ovary carcinosarcoma | 0.36% | 2 | 561 |
| Unknown primary serous carcinoma | 0.17% | 1 | 579 |
| Adrenal gland cortical carcinoma | 0.00% | 0 | 511 |
| Brain oligodendroglioma | 0.82% | 4 | 489 |
| Ampullary adenocarcinoma | 0.36% | 2 | 562 |
| Thyroid carcinoma (NOS) | 0.19% | 1 | 527 |
| Thyroid anaplastic carcinoma | 0.41% | 2 | 492 |
| Head and neck adenoid cystic carcinoma | 0.23% | 1 | 432 |
| Stomach gist | 0.00% | 0 | 473 |
| Neuroblastoma | 0.00% | 0 | 427 |
| Ovary granulosa cell tumor | 0.23% | 1 | 441 |
| Vulva squamous cell carcinoma (SCC) | 0.22% | 1 | 458 |
| Skin merkel cell carcinoma | 1.44% | 6 | 417 |
| Soft tissue synovial sarcoma | 0.47% | 2 | 427 |
| Salivary gland carcinoma (NOS) | 0.48% | 2 | 416 |
| Lung adenosquamous carcinoma | 0.70% | 3 | 431 |
| Salivary gland adenoid cystic carcinoma | 0.25% | 1 | 403 |
| Soft tissue ewing sarcoma | 0.00% | 0 | 414 |
| Soft tissue angiosarcoma | 0.73% | 3 | 413 |
| Lung sarcomatoid carcinoma | 0.25% | 1 | 408 |
| Ureter urothelial carcinoma | 0.00% | 0 | 412 |
| Soft tissue sarcoma undifferentiated | 0.77% | 3 | 392 |
| Unknown primary neuroendocrine tumor | 0.48% | 2 | 421 |
| Colon neuroendocrine carcinoma | 0.55% | 2 | 366 |
| Unknown primary undifferentiated small cell carcinoma | 0.26% | 1 | 385 |
| Stomach adenocarcinoma diffuse type | 0.90% | 3 | 333 |
| Prostate neuroendocrine carcinoma | 0.26% | 1 | 379 |
| Breast metaplastic carcinoma | 0.56% | 2 | 356 |
| Thymus carcinoma | 0.00% | 0 | 335 |
| Kidney renal papillary carcinoma | 0.00% | 0 | 352 |
| Brain astrocytoma pilocytic | 0.00% | 0 | 383 |
| Uterus endometrial adenocarcinoma clear cell | 0.54% | 2 | 373 |
| Acute leukemia (NOS) | 0.00% | 0 | 377 |
| Soft tissue malignant peripheral nerve sheath tumor (MPNST) | 0.86% | 3 | 349 |
| Eye intraocular melanoma | 0.00% | 0 | 326 |
| Salivary gland adenocarcinoma | 0.33% | 1 | 305 |
| Brain medulloblastoma | 0.00% | 0 | 323 |
| Peritoneum mesothelioma | 0.00% | 0 | 302 |
| Follicular lymphoma | 2.42% | 8 | 330 |
| Thyroid medullary carcinoma | 0.00% | 0 | 326 |
| Bladder carcinoma (NOS) | 0.00% | 0 | 316 |
| Esophagus carcinoma (NOS) | 0.00% | 0 | 316 |
| Small intestine gist | 0.00% | 0 | 287 |
| Myelodysplastic-myeloproliferative neoplasm (MDS-MPN) (NOS) | 0.00% | 0 | 308 |
| Rhabdomyosarcoma (NOS) | 0.00% | 0 | 278 |
| Soft tissue fibromatosis | 0.37% | 1 | 272 |
| Uterus endometrial stromal sarcoma | 0.00% | 0 | 283 |
| Soft tissue chondrosarcoma | 0.36% | 1 | 281 |
| Brain ependymoma | 0.00% | 0 | 266 |
| Thymus thymoma (NOS) | 0.40% | 1 | 252 |
| Mantle cell lymphoma | 0.00% | 0 | 272 |
| T-cell neoplasm (NOS) | 0.78% | 2 | 257 |
| Skin basal cell carcinoma | 2.55% | 6 | 235 |
| B-cell neoplasm (NOS) | 1.26% | 3 | 238 |
| Bone chondrosarcoma | 0.00% | 0 | 225 |
| Penis squamous cell carcinoma (SCC) | 0.00% | 0 | 232 |
| Soft tissue myxofibrosarcoma | 0.41% | 1 | 241 |
| Prostate undifferentiated carcinoma | 0.44% | 1 | 228 |
| Bladder adenocarcinoma | 0.47% | 1 | 211 |
| Nasopharynx and paranasal sinuses undifferentiated carcinoma | 0.50% | 1 | 202 |
| Unknown primary leiomyosarcoma | 0.44% | 1 | 229 |
| Acute t-lymphoblastic leukemia-lymphoma (T-ALL) | 0.00% | 0 | 229 |
| Bone chordoma | 0.00% | 0 | 200 |
| Lung large cell carcinoma | 0.48% | 1 | 210 |
| Thyroid follicular carcinoma | 0.00% | 0 | 205 |
| Lung atypical carcinoid | 0.00% | 0 | 192 |
| Uterus sarcoma (NOS) | 1.40% | 3 | 215 |
| Skin adnexal carcinoma | 3.03% | 6 | 198 |
| Soft tissue solitary fibrous tumor | 0.00% | 0 | 197 |
| Appendix mucinous neoplasm | 0.50% | 1 | 200 |
| Ovary mucinous carcinoma | 0.00% | 0 | 190 |
| Adrenal gland neuroblastoma | 0.00% | 0 | 169 |
| Chronic myelogenous leukemia (CML) | 0.00% | 0 | 205 |
| Appendix goblet cell carcinoid (GCC) | 0.53% | 1 | 190 |
| Unknown primary adenoid cystic carcinoma | 0.54% | 1 | 185 |
| Unknown primary sarcomatoid carcinoma | 1.08% | 2 | 186 |
| Salivary gland duct carcinoma | 0.55% | 1 | 183 |
| Ovary carcinoma mixed histology | 0.00% | 0 | 189 |
| Salivary gland acinic cell tumor | 0.00% | 0 | 159 |
| Uterus endometrial adenocarcinoma mixed histology | 1.07% | 2 | 187 |
| Testis germ cell tumor (non-seminoma) | 0.60% | 1 | 168 |
| Bladder squamous cell carcinoma (SCC) | 1.16% | 2 | 173 |
| Kidney carcinoma (NOS) | 0.66% | 1 | 151 |
| Pancreas acinar cell carcinoma | 0.00% | 0 | 171 |
| Unknown primary carcinoid | 0.00% | 0 | 155 |
| Head and neck melanoma | 0.63% | 1 | 160 |
| Vagina squamous cell carcinoma (SCC) | 0.63% | 1 | 158 |
| Nasopharynx and paranasal sinuses squamous cell carcinoma (SCC) | 0.00% | 0 | 154 |
| Small intestine neuroendocrine carcinoma | 0.00% | 0 | 157 |
| Kidney sarcomatoid carcinoma | 0.70% | 1 | 142 |
| Head and neck carcinoma (NOS) | 0.00% | 0 | 141 |
| Stomach adenocarcinoma intestinal type | 0.78% | 1 | 129 |
| Soft tissue desmoplastic small round cell tumor | 1.43% | 2 | 140 |
| Anus melanoma | 0.00% | 0 | 145 |
| Prostate ductal adenocarcinoma | 0.00% | 0 | 144 |
| Kidney Wilms tumor | 0.71% | 1 | 141 |
| Rectum squamous cell carcinoma (SCC) | 0.00% | 0 | 136 |
| Fallopian tube adenocarcinoma | 0.00% | 0 | 133 |
| Bone marrow failure (NOS) | 0.00% | 0 | 143 |
| Ovary low grade serous carcinoma | 0.00% | 0 | 143 |
| Soft tissue fibrosarcoma | 0.00% | 0 | 123 |
| Small intestine carcinoid | 0.00% | 0 | 126 |
| Lung adenoid cystic carcinoma | 0.00% | 0 | 119 |
| Soft tissue rhabdomyosarcoma alveolar | 0.00% | 0 | 130 |
| Paraganglioma | 0.00% | 0 | 119 |
| Soft tissue rhabdomyosarcoma embryonal | 0.00% | 0 | 118 |
| Cervix neuroendocrine carcinoma | 0.00% | 0 | 113 |
| Salivary gland mucoepidermoid carcinoma | 0.00% | 0 | 115 |
| Peritoneum adenocarcinoma | 0.00% | 0 | 113 |
| Brain gliosarcoma | 0.83% | 1 | 120 |
| Breast angiosarcoma | 0.85% | 1 | 117 |
| Langerhans cell histiocytosis (LCH) | 0.00% | 0 | 118 |
| Brain anaplastic oligodendroglioma | 0.85% | 1 | 117 |
| Non-hodgkin lymphoma (NOS) | 0.92% | 1 | 109 |
| Brain ganglioglioma | 0.00% | 0 | 106 |
| Bladder small cell carcinoma | 0.00% | 0 | 106 |
| Esophagus neuroendocrine carcinoma | 0.00% | 0 | 104 |
| Lymphoplasmacytic lymphoma-Waldenstroms macroglobulinemia | 0.00% | 0 | 105 |

Interestingly, three of the five tumor types with the highest frequency of CD274 mutations usually occur on the skin. When the mean TMB for these cases was examined, very high mean TMB and median TMB was observed (cutaneous squamous cell carcinoma [151 mut/Mb, 100 mut/Mb], cutaneous melanoma [133 mut/Mb, 126 mut/Mb], unknown primary melanoma [125 mut/Mb, 92 mut/Mb], respectively), suggesting that these were likely caused by ultraviolet exposure-induced hypermutation. This was further supported by the high prevalence of ultraviolet mutational signature in these tumor types (cutaneous squamous cell carcinoma [84.6%, 11/13], cutaneous melanoma [93.8%, 30/32], and unknown primary melanoma [100%, 32/32]). It is unknown whether these CD274 mutations are random “passenger” mutations due to the high mutation rate.

Correlation of CD274 Mutations with PD-L1 IHC Tumor Cell Expression and Predicted Functionality Models

Of the 1,081 cases with CD274 mutations, 19.7% (213/1,081) of cases had PD-L1 IHC data; and of the 313,550 cases without CD274 mutations, 18.6% (58,218/313,550) of cases had PD-L1 IHC data.

Most of the CD274 non-truncating mutations had low to no tumor cell expression of PD-L1 (see, FIG. 4A and Table 11).

TABLE 11
Age, Sex, Diagnosis and PD-LI Tumor Cell Expression of CD274 Mutations.

| | | | | | |
|---|---|---|---|---|---|
| Case | Genomic | PD-L1 | | | |
| ID | Alteration | TPS | Diagnosis | Age | Sex |

Missense Mutations

| | | | | | |
|---|---|---|---|---|---|
| 1 | A109V | 5 | Pancreas ductal adenocarcinoma | 63 | Male |
| 2 | A157S | 0 | Unknown primary adenocarcinoma | 67 | Female |
| 3 | A163V | 5 | Soft tissue angiosarcoma | 75 | Male |
| 4 | A18S | 0 | Colon adenocarcinoma | 63 | Male |
| 5 | A18T | 0 | Lung adenocarcinoma | 72 | Female |
| 6 | A18T | 1 | Unknown primary carcinoma | 34 | Female |
| 7 | A222V | 60 | Bile duct adenocarcinoma | 76 | Male |
| 8 | A232S | 0 | Prostate acinar adenocarcinoma | 74 | Male |
| 9 | A254G | 1 | Ovary serous carcinoma | 74 | Female |
| 10 | A85S | 0 | Liver hepatocellular carcinoma | 56 | Male |
| 11 | A85V | 0 | Uterus endometrial adenocarcinoma | 63 | Female |
| | | | endometrioid | | |
| 12 | A97V | 0 | Skin squamous cell carcinoma | 58 | Male |
| 13 | C209S | 100 | Lung adenocarcinoma | 67 | Male |
| 14 | C272Y | 0 | Colon adenocarcinoma | 58 | Female |
| 15 | D122N | 30 | Uterus endometrial adenocarcinoma | 54 | Female |
| | | | mixed histology | | |
| 16 | D145Y | 0 | Thyroid anaplastic carcinoma | 70 | Male |
| 17 | D171G | 0 | Rectum adenocarcinoma | 34 | Male |
| 18 | D171N | 0 | Head and neck squamous cell carcinoma | 54 | Female |
| 19 | D215H | 0 | Colon adenocarcinoma | 68 | Female |
| 20 | D215H | 0 | Pancreas adenosquamous carcinoma | 71 | Male |
| 21 | D276E | 0 | Ovary serous carcinoma | 80 | Female |
| 22 | D284N | 5 | Skin melanoma | 71 | Female |
| 23 | D61Y | 1 | Liver hepatocellular carcinoma | 59 | Female |
| 24 | E187D | 25 | Unknown primary squamous cell | 82 | Male |
| | | | carcinoma | | |
| 25 | E187D | 0 | Uterus endometrial adenocarcinoma | 57 | Female |
| | | | endometrioid | | |
| 26 | E187Q | 1 | Lung adenocarcinoma | 55 | Female |
| 27 | E187V | 0 | Lung adenocarcinoma | 55 | Male |
| 28 | E188Q | 5 | Lung adenocarcinoma | 62 | Female |
| 29 | E205Q | 50 | Unknown primary adenocarcinoma | 81 | Male |
| 30 | E217K | 0 | Pancreas ductal adenocarcinoma | 85 | Male |
| 31 | E217Q | 0 | Lung squamous cell carcinoma | 66 | Female |
| 32 | E218K | 60 | Lung adenocarcinoma | 57 | Female |
| 33 | E223K | 90 | Lung adenocarcinoma | 72 | Female |
| 34 | E223K | 20 | Lung adenocarcinoma | 71 | Female |
| 35 | E223K | 30 | Unknown primary squamous cell | 70 | Female |
| | | | carcinoma | | |
| 36 | E228G | 0 | Colon adenocarcinoma | 40 | Female |
| 37 | E228V | 0 | Colon adenocarcinoma | 60 | Male |
| 38 | E237K | 50 | Lung adenocarcinoma | 60 | Male |
| 39 | E237K | 0 | Breast invasive ductal carcinoma | 34 | Female |
| 40 | E58K | 50 | Skin melanoma | 69 | Male |
| 41 | E71Q | 0 | Lung adenocarcinoma | 61 | Male |
| 42 | F211C | 0 | Colon adenocarcinoma | 74 | Female |
| 43 | F257L | 0 | Uterus endometrial adenocarcinoma | 53 | Female |
| | | | mixed histology | | |
| 44 | F9L | 1 | Uterus endometrial adenocarcinoma | 57 | Female |
| 45 | G110E | 0 | Colon adenocarcinoma | 66 | Male |
| 46 | G177D | 0 | Colon adenocarcinoma | 76 | Female |
| 47 | G177S | 0 | Skin melanoma | 72 | Male |
| 48 | G177V | 0 | Duodenum adenocarcinoma | 53 | Male |
| 49 | G245V | 0 | Uterus endometrial adenocarcinoma | 54 | Female |
| | | | papillary serous | | |
| 50 | G252S | 0 | Lung non-small cell lung carcinoma | 66 | Male |
| 51 | G264W | 0 | Colon adenocarcinoma | 44 | Male |
| 52 | H14Y | 0 | Lung adenocarcinoma | 70 | Female |
| 53 | H151L | 0 | Colon adenocarcinoma | 63 | Male |
| 54 | H172Y | 0 | Lung adenocarcinoma | 75 | Male |
| 55 | H220Y | 0 | Skin melanoma | 54 | Female |
| 56 | H220Y | 0 | Fallopian tube carcinosarcoma | 62 | Female |
| 57 | H233Y | 0 | Unknown primary neuroendocrine tumor | 62 | Female |
| 58 | H78R | 1 | Lung adenocarcinoma | 75 | Male |
| 59 | I126S | 0 | Colon adenocarcinoma | 41 | Female |
| 60 | I166L | 1 | Lung squamous cell carcinoma | 83 | Male |
| 61 | I199V | 5 | Lung adenocarcinoma | 62 | Male |
| 62 | I206M | 50 | Uterus endometrial adenocarcinoma | 41 | Female |
| 63 | I243T | 0 | Colon adenocarcinoma | 49 | Female |
| 64 | I258V | 70 | Unknown primary carcinoma | 61 | Female |
| 65 | I38M | 70 | Breast carcinoma | 66 | Female |
| 66 | I64T | 90 | Lung adenocarcinoma | 54 | Female |
| 67 | I8K | 0 | Lung adenocarcinoma | 76 | Male |
| 68 | K105Q | 100 | Lung adenocarcinoma | 85 | Male |
| 69 | K105Q | 50 | Breast carcinoma | 66 | Female |
| 70 | K105Q | 0 | Unknown primary undifferentiated small | 71 | Male |
| | | | cell carcinoma | | |
| 71 | K162Q | 1 | Head and neck squamous cell carcinoma | 80 | Male |
| 72 | K178E | 0 | Salivary gland adenoid cystic carcinoma | 58 | Female |
| 73 | K25N | 0 | Uterus endometrial adenocarcinoma | 62 | Female |
| | | | papillary serous | | |
| 74 | K280N | 0 | Uterus carcinosarcoma | 57 | Female |
| 75 | K280N | 0 | Lung adenocarcinoma | 63 | Male |
| 76 | K75T | 90 | Lung adenocarcinoma | 67 | Male |
| 77 | L142F | 0 | Lung adenocarcinoma | 85 | Male |
| 78 | L190V | 0 | Lung small cell undifferentiated | 56 | Female |
| | | | carcinoma | | |
| 79 | L197P | 10 | Colon adenocarcinoma | 55 | Female |
| 80 | L50V | 1 | Colon adenocarcinoma | 84 | Male |
| 81 | L92H | 20 | Lung squamous cell carcinoma | 65 | Male |
| 82 | M1I | 5 | Lung squamous cell carcinoma | 60 | Female |
| 83 | M1I | 0 | Lung squamous cell carcinoma | 76 | Female |
| 84 | M266I | 10 | Lung squamous cell carcinoma | 54 | Male |
| 85 | M267I | 50 | Unknown primary melanoma | 77 | Male |
| 86 | M59I | 0 | Colon adenocarcinoma | 74 | Female |
| 87 | M59I | 0 | Uterus endometrial adenocarcinoma | 68 | Female |
| 88 | N135S | 0 | Breast carcinoma | 31 | Female |
| 89 | N135S | 0 | Lung adenosquamous carcinoma | 72 | Female |
| 90 | N183S | 0 | Skin basal cell carcinoma | 65 | Female |
| 91 | N183S | 0 | Skin melanoma | 69 | Male |
| 92 | N200D | 0 | Prostate acinar adenocarcinoma | 75 | Male |
| 93 | N96S | 0 | Uterus endometrial adenocarcinoma | 58 | Female |
| | | | endometrioid | | |
| 94 | N96Y | 0 | Anus squamous cell carcinoma | 70 | Male |
| 95 | P133S | 0 | Prostate acinar adenocarcinoma | 64 | Male |
| 96 | P146S | 0 | Skin melanoma | 80 | Female |
| 97 | P216H | 2 | Skin merkel cell carcinoma | 72 | Male |
| 98 | P216S | 50 | Skin melanoma | 77 | Male |
| 99 | P230S | 5 | Lung adenocarcinoma | 63 | Male |
| 100 | P234R | 50 | Lung adenocarcinoma | 71 | Male |
| 101 | P235L | 30 | Soft tissue angiosarcoma | 76 | Male |
| 102 | P24A | 60 | Thyroid carcinoma | 81 | Male |
| 103 | P24S | 0 | Soft tissue sarcoma | 64 | Male |
| 104 | P43A | 1 | Breast invasive ductal carcinoma | 39 | Female |
| 105 | P43L | 10 | Skin melanoma | 64 | Male |
| 106 | P43Q | 1 | Bladder adenocarcinoma | 60 | Male |
| 107 | Q100R | 0 | Uterus endometrial adenocarcinoma | 60 | Female |
| 108 | Q107K | 0 | Prostate acinar adenocarcinoma | 81 | Male |
| 109 | Q107K | 50 | Pancreas ductal adenocarcinoma | 75 | Male |
| 110 | Q156H | 0 | Uterus endometrial adenocarcinoma | 55 | Female |
| 111 | Q156K | 90 | Lung non-small cell lung carcinoma | 60 | Male |
| 112 | Q173E | 0 | Uterus endometrial adenocarcinoma | 61 | Female |
| | | | papillary serous | | |
| 113 | Q173H | 0 | Colon adenocarcinoma | 56 | Female |
| 114 | Q275R | 0 | Lung non-small cell lung carcinoma | 61 | Female |
| 115 | Q83H | 0 | Lung adenocarcinoma | 82 | Male |
| 116 | Q83H | 0 | Fallopian tube serous carcinoma | 71 | Female |
| 117 | Q83H | 1 | Lung small cell undifferentiated | 80 | Male |
| | | | carcinoma | | |
| 118 | R113H | 0 | Colon adenocarcinoma | 74 | Male |
| 119 | R113H | 0 | Colon adenocarcinoma (CRC) | 68 | Female |
| 120 | R125Q | 0 | Colon adenocarcinoma (CRC) | 55 | Male |
| 121 | R125Q | 0 | Skin squamous cell carcinoma (SCC) | 71 | Female |
| 122 | R125Q | 0 | Rectum adenocarcinoma (CRC) | 49 | Male |
| 123 | R125Q | 1 | Skin melanoma | 89 | Male |
| 124 | R140I | 0 | Colon adenocarcinoma (CRC) | 45 | Male |
| 125 | R140I | 0 | Colon adenocarcinoma (CRC) | 26 | Female |
| 126 | R140T | 99 | Lung non-small cell lung carcinoma | 58 | Female |
| | | | (NSCLC) (NOS) | | |
| 127 | R140T | 5 | Lung adenocarcinoma | 65 | Female |
| 128 | R186T | 60 | Lung adenocarcinoma | 69 | Male |
| 129 | R198K | 80 | Lung adenocarcinoma | 75 | Female |
| 130 | R198T | 0 | Lung adenocarcinoma | 71 | Female |
| 131 | R212K | 0 | Prostate acinar adenocarcinoma | 79 | Male |
| 132 | R260C | 0 | Prostate acinar adenocarcinoma | 82 | Male |
| 133 | R260H | 0 | Colon adenocarcinoma | 76 | Female |
| 134 | R260H | 0 | Prostate acinar adenocarcinoma | 56 | Male |
| 135 | R260H | 0 | Lung adenocarcinoma | 68 | Male |
| 136 | R260H | 0 | Colon adenocarcinoma | 55 | Male |
| 137 | R260H | 1 | Ampullary adenocarcinoma | 49 | Male |
| 138 | R260H | 1 | Brain glioblastoma | 49 | Male |
| 139 | R260H | 0 | Prostate acinar adenocarcinoma | 59 | Male |
| 140 | R260H | 0 | Ovary epithelial carcinoma | 44 | Female |
| 141 | R260H | 0 | Breast carcinoma | 56 | Female |
| 142 | R260H | 0 | Uterus endometrial adenocarcinoma | 63 | Female |
| | | | papillary serous | | |
| 143 | R260H | 0 | Rectum adenocarcinoma | 89 | Male |
| 144 | R2M | 0 | Lung non-small cell lung carcinoma | 44 | Male |
| 145 | R82I | 0 | Unknown primary carcinoma | 62 | Male |
| 146 | R86W | 0 | Ovary carcinosarcoma | 59 | Female |
| 147 | R86W | 0 | Colon adenocarcinoma | 74 | Male |
| 148 | S195R | 5 | Unknown primary squamous cell | 80 | Male |
| | | | carcinoma | | |
| 149 | T148A | 70 | Pancreas ductal adenocarcinoma | 72 | Male |
| 150 | T180I | 0 | Colon adenocarcinoma | 75 | Male |
| 151 | T181I | 5 | Lung adenocarcinoma | 62 | Male |
| 152 | T182P | 90 | Lung adenocarcinoma | 78 | Female |
| 153 | T196A | 0 | Lung squamous cell carcinoma | 69 | Male |
| 154 | T201I | 0 | Lung squamous cell carcinoma | 78 | Male |
| 155 | T290A | 1 | Gallbladder adenocarcinoma | 67 | Female |
| 156 | T37A | 0 | Colon adenocarcinoma | 57 | Male |
| 157 | T37K | 5 | Fallopian tube serous carcinoma | 68 | Female |
| 158 | V130L | 0 | Ovary epithelial carcinoma | 76 | Female |
| 159 | V143A | 0 | Lung adenocarcinoma | 88 | Male |
| 160 | V147A | 70 | Pancreas ductal adenocarcinoma | 52 | Female |
| 161 | V21A | 0 | Uterus endometrial adenocarcinoma | 52 | Female |
| 162 | V21L | 1 | Lung adenocarcinoma | 63 | Male |
| 163 | V253I | 0 | Pancreas ductal adenocarcinoma | 77 | Male |
| 164 | V30A | 0 | Breast metaplastic carcinoma | 46 | Female |
| 165 | V6I | 1 | Unknown primary sarcoma | 53 | Male |
| 166 | V6I | 0 | Vagina squamous cell carcinoma | 65 | Female |
| 167 | V76F | 0 | Lung adenocarcinoma | 52 | Female |
| 168 | W13C | 50 | Lung large cell carcinoma | 73 | Female |
| 169 | W57G | 0 | Pancreas ductal adenocarcinoma | 65 | Male |
| 170 | Y112C | 0 | Duodenum adenocarcinoma | 54 | Male |
| 171 | Y134C | 0 | Lung adenocarcinoma | 52 | Male |
| 172 | Y160H | 99 | Kidney sarcomatoid carcinoma | 48 | Female |
| 173 | Y160H | 0 | Pancreas ductal adenocarcinoma | 48 | Female |
| 174 | Y28S | 0 | Skin melanoma | 75 | Male |
| 175 | Y32C | 10 | Lung squamous cell carcinoma | 75 | Male |
| 176 | Y81H | 0 | Prostate acinar adenocarcinoma | 76 | Male |

Truncations

| | | | | | |
|---|---|---|---|---|---|
| 177 | *291Yext*42 | 0 | Colon adenocarcinoma | 29 | Male |
| 178 | C272fs*13 | 0 | Uterus endometrial adenocarcinoma | 68 | Female |
| 179 | C272fs*13 | 0 | Ovary serous carcinoma | 50 | Female |
| 180 | D90fs*10 | 0 | Prostate acinar adenocarcinoma | 73 | Male |
| 181 | E150* | 80 | Lung adenocarcinoma | 72 | Female |
| 182 | E152* | 0 | Uterus endometrial adenocarcinoma | 62 | Female |
| | | | endometrioid | | |
| 183 | E152* | 1 | Diffuse large b-cell lymphoma | 87 | Female |
| 184 | E188fs*12 | 0 | Ovary epithelial carcinoma | 69 | Female |
| 185 | E217* | 20 | Lung non-small cell lung carcinoma | 58 | Female |
| 186 | F7fs*27 | 0 | Colon adenocarcinoma | 71 | Female |
| 187 | K271fs*44 | 1 | Uterus endometrial adenocarcinoma | 50 | Female |
| 188 | K271fs*44 | 0 | Skin adnexal carcinoma | 46 | Male |
| 189 | K41* | 0 | Colon adenocarcinoma | 45 | Female |
| 190 | P133fs*21 | 0 | Lung adenocarcinoma | 71 | Male |
| 191 | Q107* | 0 | Ovary serous carcinoma | 72 | Female |
| 192 | Q66fs*13 | 70 | Lung adenosquamous carcinoma | 84 | Male |
| 193 | Q77* | 0 | Lung adenocarcinoma | 60 | Female |
| 194 | R125* | 5 | Uterus endometrial adenocarcinoma | 80 | Female |
| | | | endometrioid | | |
| 195 | R125* | 0 | Brain glioblastoma | 18 | Female |
| 196 | R125* | 0 | Prostate undifferentiated carcinoma | 82 | Male |
| 197 | R265fs*2 | 0 | Prostate acinar adenocarcinoma | 64 | Male |
| 198 | S279* | 5 | Lung squamous cell carcinoma | 72 | Female |
| 199 | Y134* | 0 | Pancreas ductal adenocarcinoma | 61 | Female |

Splice Site Mutations

| | | | | | |
|---|---|---|---|---|---|
| 200 | splice site | 70 | Lung adenocarcinoma | 79 | Female |
| | 53 − 49_82del79 | | | | |
| 201 | splice site | 0 | Colon adenocarcinoma | 77 | Male |
| | 683 − 1G > A | | | | |
| 202 | splice site | 30 | Rectum adenocarcinoma | 76 | Female |
| | 790 + 1_790 + | | | | |
| | 4delGTAG | | | | |
| 203 | splice site | 0 | Lung adenocarcinoma | 81 | Male |
| | 790 + 1G > T | | | | |
| 204 | splice site | 0 | Skin melanoma | 65 | Male |
| | 791 − 1G > A | | | | |
| 205 | splice site | 0 | Colon adenocarcinoma | 51 | Female |
| | 791 − 1G > T | | | | |
| 206 | splice site | 0 | Lung squamous cell carcinoma | 73 | Male |
| | 791 − 1G > T | | | | |

Insertion/Deletions

| | | | | | |
|---|---|---|---|---|---|
| 207 | E31_Y32insFTVTVPKDLYVVE | 0 | Thyroid papillary carcinoma | 27 | Male |
| 208 | T203del | 40 | Breast invasive ductal carcinoma | 50 | Female |

Missense Mutations with CD274 amplification

| | | | | | |
|---|---|---|---|---|---|
| 209 | E150G | 99 | Lung adenocarcinoma | 69 | Male |
| 210 | V242I | 1 | Breast carcinoma | 44 | Female |
| 211 | K25R | 40 | Unknown primary squamous cell | 82 | Male |
| | | | carcinoma | | |

Multiple Missense Mutations

| | | | | | |
|---|---|---|---|---|---|
| 212 | I141M, F9L | 0 | Uterus endometrial adenocarcinoma | 49 | Female |
| | | | endometrioid | | |
| 213 | K189M, E188K | 0 | Colon neuroendocrine carcinoma | 51 | Male |

Of the eleven R260H cases concurrently tested with PD-L1 IHC, 81.8% (9/11) had no PD-L1 expression and two cases (18.2%) had low PD-L1 expression. Of the five E237K cases, 80% (4/5) had PL-L1 expression (20% (1/5) had no PD-L1 expression, 40% (2/5) had low PD-L1 expression, and 40% (2/5) had high PD-L1 expression; see, FIG. 4B). This difference in protein expression of the two mutations was significantly different (Fisher's Exact Test, p=0.036). When examining the PD-L1 protein expression of cases with CD274 missense mutations (n=153) and cases without CD274 mutations (n=58,218), a lower level of PD-L1 IHC expression in the cases with CD274 mutations was observed, though the difference was not statistically significant (mean: TPS=11 vs 13, respectively; ANOVA, p=0.404). When examining the clonal (n=153) versus sub-clonal (n=28) missense mutations, a significantly lower PD-L1 IHC expression in the clonal missense mutations was observed (mean: TPS=11 versus 22, respectively; ANOVA, p=0.049). See, FIG. 4C.

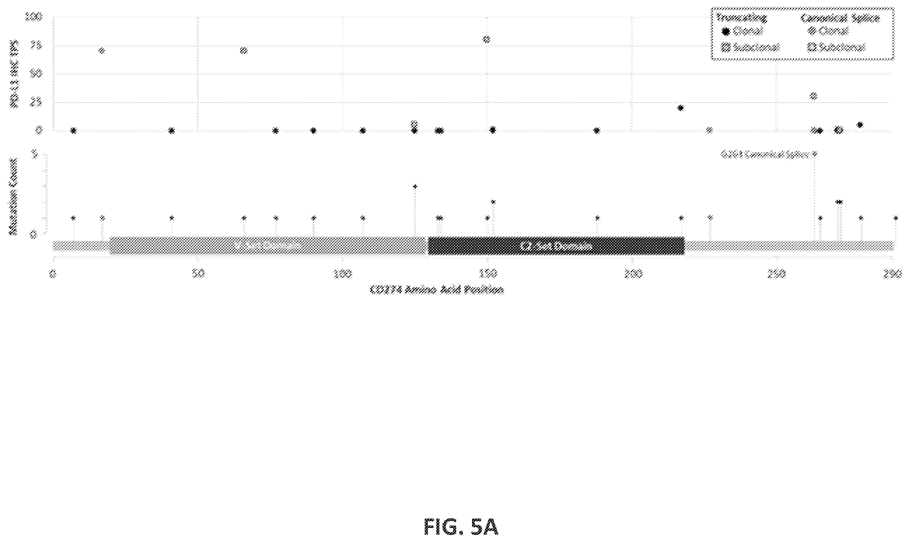

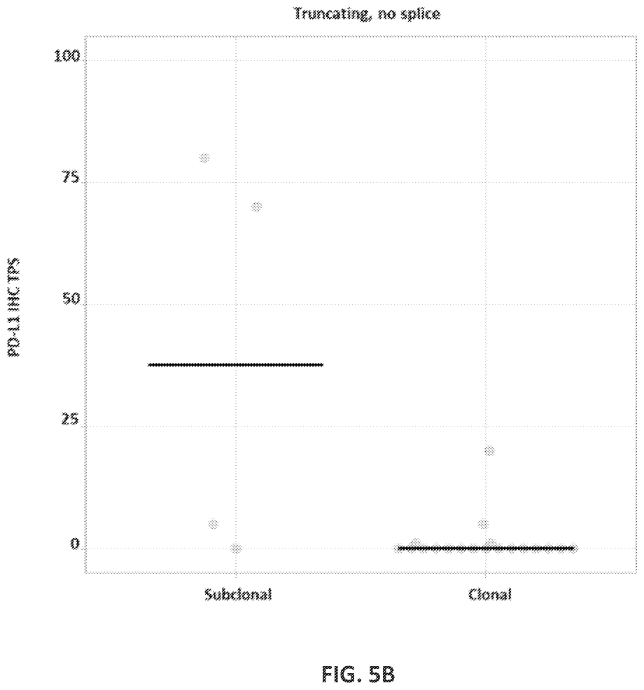

Thirty-nine putative truncating variants with concurrent PD-L1 IHC testing were identified, including 12 nonsense mutations, 10 frame shift indels, and seven canonical splice variants (see, FIG. 5A). Comparison of the PD-L1 protein expression of cases with CD274 clonal truncating variants (nonsense or frame shift indel) (n=18) and cases without CD274 mutations (n=58218) revealed a lower level of PD-L1 expression in the cases with CD274 mutations (mean: TPS=1 versus 13, respectively; ANOVA, p=0.069). In addition, a significantly lower level of PD-L1 expression in samples with clonal truncating variants (nonsense or frame shift indel) when compared to samples with sub-clonal truncating variants was observed (mean: TPS=1 versus TPS=38; ANOVA, p<0.001). See, FIG. 5B.

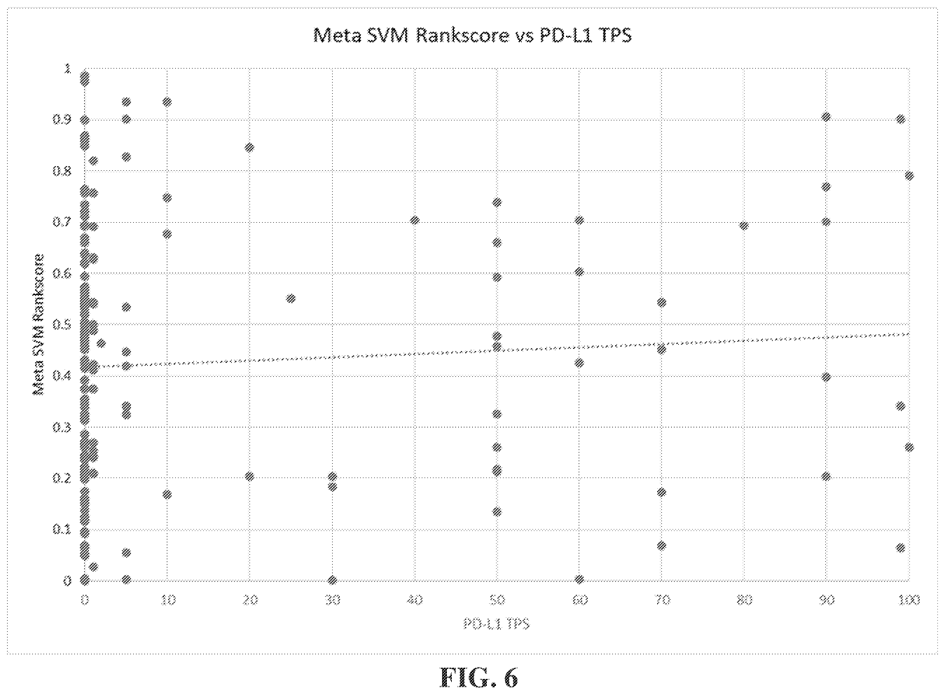

Finally, the predicted functionality of each CD274 missense mutation was analyzed with multiple functionality prediction models including SIFT, MutationTaster, fathmm-MKL, and MetaSVM (Table 12). However, the predicted functionality did not have any significant correlation with PD-L1 IHC expression (FIG. 6).

TABLE 12
SIFT Converted Rankscore, MutationTaster Converted
Rankscore, Fathmm-MKL Coding Rankscore, and MetaSVM
Rankscore for CD274 Missense Mutations.

| | SIFT | Mutation Taster | fathmm-MKL | |
|---|---|---|---|---|
| Missense | converted | converted | coding | MetaSVM |
| Mutation | rankscore | rankscore | rankscore | rankscore |
| Y112C | 0.91255 | 0.81001 | 0.76812 | 0.98514 |
| W57G | 0.72154 | 0.81001 | 0.73721 | 0.97448 |
| W57S | 0.72154 | 0.81001 | 0.8128 | 0.97206 |
| D108N | 0.91255 | 0.81001 | 0.76804 | 0.95276 |
| L197R | 0.91255 | 0.3084 | 0.37232 | 0.93982 |
| S195R | 0.91255 | 0.52396 | 0.56437 | 0.93462 |
| L197P | 0.91255 | 0.52935 | 0.35894 | 0.93462 |
| S195I | 0.91255 | 0.53665 | 0.57529 | 0.93317 |
| S195N | 0.91255 | 0.49394 | 0.55559 | 0.93317 |
| G33C | 0.91255 | 0.81001 | 0.77901 | 0.92711 |
| G159R | 0.91255 | 0.58761 | 0.57613 | 0.92475 |
| G159C | 0.91255 | 0.58761 | 0.56798 | 0.92475 |
| L190R | 0.91255 | 0.49283 | 0.51634 | 0.91321 |
| Q156E | 0.68238 | 0.36075 | 0.39841 | 0.91069 |
| Q156K | 0.68238 | 0.37557 | 0.40233 | 0.90642 |
| A163V | 0.91255 | 0.4977 | 0.49944 | 0.90143 |
| V165A | 0.72154 | 0.49283 | 0.49098 | 0.90102 |
| E150G | 0.7849 | 0.47904 | 0.53381 | 0.90102 |
| T194A | 0.65419 | 0.43243 | 0.55584 | 0.90026 |
| V193M | 0.7849 | 0.46249 | 0.46499 | 0.89995 |
| T202I | 0.24183 | 0.3397 | 0.28723 | 0.88977 |
| E150K | 0.46129 | 0.46733 | 0.3828 | 0.87947 |
| E158V | 0.65419 | 0.43214 | 0.45412 | 0.87733 |
| N192I | 0.68238 | 0.4367 | 0.422 | 0.87567 |
| V165L | 0.65419 | 0.4977 | 0.57613 | 0.87208 |
| N200D | 0.31088 | 0.41047 | 0.45378 | 0.86989 |
| E158Q | 0.51853 | 0.41402 | 0.49575 | 0.86669 |
| L190V | 0.65419 | 0.3075 | 0.3095 | 0.86503 |
| Q156H | 0.7849 | 0.38482 | 0.40744 | 0.85799 |
| E164Q | 0.61437 | 0.36795 | 0.50767 | 0.85181 |
| H172P | 0.54683 | 0.44019 | 0.32732 | 0.85053 |
| V21A | 0.72154 | 0.81001 | 0.61888 | 0.8488 |
| L92H | 0.91255 | 0.81001 | 0.58419 | 0.84686 |
| E158G | 0.63226 | 0.41837 | 0.46238 | 0.84675 |
| L190I | 0.61437 | 0.29766 | 0.30845 | 0.84648 |
| N200S | 0.36509 | 0.4129 | 0.47062 | 0.84386 |
| E158K | 0.26852 | 0.44492 | 0.44365 | 0.84263 |
| I166V | 0.31383 | 0.34922 | 0.469 | 0.84231 |
| K25M | 0.7849 | 0.81001 | 0.70389 | 0.84074 |
| F67C | 0.72154 | 0.81001 | 0.5832 | 0.83603 |
| E164A | 0.63226 | 0.38848 | 0.40015 | 0.83084 |
| N35H | 0.72154 | 0.81001 | 0.76619 | 0.82903 |
| A109V | 0.61437 | 0.81001 | 0.56674 | 0.82739 |
| E152Q | 0.53172 | 0.36172 | 0.41133 | 0.82641 |
| I166L | 0.20002 | 0.3662 | 0.47041 | 0.82087 |
| L190F | 0.33254 | 0.34078 | 0.26703 | 0.8005 |
| A132V | 0.91255 | 0.53665 | 0.43883 | 0.7992 |
| A132D | 0.91255 | 0.58761 | 0.48778 | 0.7992 |
| T202R | 0.02847 | 0.36155 | 0.27255 | 0.79553 |
| C209S | 0.91255 | 0.58761 | 0.51349 | 0.79147 |
| C209Y | 0.91255 | 0.81001 | 0.50542 | 0.78749 |
| C209F | 0.91255 | 0.81001 | 0.53306 | 0.78749 |
| V23F | 0.72154 | 0.81001 | 0.52807 | 0.78644 |
| T154S | 0.539 | 0.22369 | 0.46945 | 0.78626 |
| D90Y | 0.72154 | 0.81001 | 0.574 | 0.77905 |
| Y118F | 0.91255 | 0.81001 | 0.61025 | 0.77885 |
| T102I | 0.49613 | 0.81001 | 0.22685 | 0.77714 |
| T179I | 0.46129 | 0.21134 | 0.35026 | 0.77294 |
| I64T | 0.10482 | 0.81001 | 0.61815 | 0.77045 |
| Y134C | 0.91255 | 0.45228 | 0.58976 | 0.76448 |
| G119V | 0.72154 | 0.81001 | 0.51883 | 0.76156 |
| Y56C | 0.37536 | 0.81001 | 0.34772 | 0.75975 |
| A18T | 0.91255 | 0.58761 | 0.64259 | 0.75706 |
| Y32C | 0.63226 | 0.81001 | 0.28603 | 0.7479 |
| N204I | 0.68238 | 0.08975 | 0.21934 | 0.74595 |
| A97T | 0.68238 | 0.21331 | 0.333 | 0.74492 |
| S184P | 0.61437 | 0.08975 | 0.21092 | 0.74052 |
| E39Q | 0.91255 | 0.81001 | 0.78815 | 0.74012 |
| I206M | 0.33923 | 0.29246 | 0.35983 | 0.73988 |
| N35K | 0.56456 | 0.81001 | 0.50828 | 0.73777 |
| Y208C | 0.3631 | 0.36012 | 0.347 | 0.73765 |
| A97V | 0.72154 | 0.44295 | 0.41085 | 0.73478 |
| F67I | 0.45756 | 0.37165 | 0.25589 | 0.73389 |
| S184F | 0.7849 | 0.08975 | 0.16884 | 0.73305 |
| I64M | 0.56456 | 0.40254 | 0.36355 | 0.73132 |
| G119S | 0.58626 | 0.81001 | 0.65144 | 0.7304 |
| E187V | 0.41637 | 0.08975 | 0.18105 | 0.72231 |
| E39K | 0.91255 | 0.81001 | 0.75311 | 0.7215 |
| D26Y | 0.72154 | 0.81001 | 0.33467 | 0.72093 |
| K25N | 0.68238 | 0.81001 | 0.4724 | 0.72068 |
| C114Y | 0.91255 | 0.81001 | 0.76804 | 0.71932 |
| S176G | 0.72154 | 0.08975 | 0.35923 | 0.71885 |
| T201I | 0.38185 | 0.20249 | 0.22446 | 0.7119 |
| K25R | 0.50676 | 0.81001 | 0.6962 | 0.70424 |
| E218K | 0.51248 | 0.08975 | 0.31936 | 0.70377 |
| L16P | 0.7849 | 0.58761 | 0.69832 | 0.70184 |
| T182P | 0.58626 | 0.08975 | 0.22183 | 0.70044 |
| N138H | 0.68238 | 0.34504 | 0.44203 | 0.69519 |
| Y81H | 0.13131 | 0.81001 | 0.45676 | 0.69476 |
| R198K | 0.42487 | 0.18878 | 0.21852 | 0.69403 |
| Q173H | 0.59928 | 0.08975 | 0.18168 | 0.69165 |
| V21L | 0.56456 | 0.4211 | 0.21458 | 0.69165 |
| N204K | 0.34444 | 0.08975 | 0.16465 | 0.68772 |
| G119D | 0.53172 | 0.81001 | 0.42135 | 0.68686 |
| D26G | 0.61437 | 0.81001 | 0.41215 | 0.68553 |
| Q91H | 0.68238 | 0.81001 | 0.39276 | 0.68225 |
| R198G | 0.42783 | 0.20581 | 0.23393 | 0.67903 |
| P43L | 0.61437 | 0.81001 | 0.5397 | 0.67598 |
| T154I | 0.24468 | 0.19486 | 0.15485 | 0.67349 |
| E187K | 0.26965 | 0.08975 | 0.14993 | 0.67001 |
| R198T | 0.25154 | 0.20372 | 0.22225 | 0.66874 |
| T210S | 0.46129 | 0.26469 | 0.33782 | 0.66503 |
| E205Q | 0.45039 | 0.20412 | 0.33325 | 0.65967 |
| A18S | 0.91255 | 0.81001 | 0.55559 | 0.65943 |
| H78Y | 0.68238 | 0.81001 | 0.33507 | 0.65791 |
| D73H | 0.539 | 0.81001 | 0.55123 | 0.65312 |
| D90A | 0.5748 | 0.81001 | 0.52663 | 0.65004 |
| R212T | 0.2708 | 0.08975 | 0.2651 | 0.64718 |
| P133L | 0.72154 | 0.58761 | 0.53491 | 0.64714 |
| E288Q | 0.91255 | 0.32163 | 0.44924 | 0.64449 |
| A157S | 0.16793 | 0.39959 | 0.32222 | 0.64005 |
| V68M | 0.42783 | 0.27403 | 0.29081 | 0.63798 |
| P146S | 0.5748 | 0.35854 | 0.32338 | 0.63723 |
| L27I | 0.49117 | 0.20152 | 0.16223 | 0.63418 |
| P43A | 0.46129 | 0.81001 | 0.6514 | 0.63018 |
| E187Q | 0.21385 | 0.08975 | 0.16164 | 0.62797 |
| Q139E | 0.28148 | 0.38806 | 0.41654 | 0.62749 |
| H172Y | 0.20306 | 0.39164 | 0.23517 | 0.62072 |
| T196A | 0.289 | 0.21485 | 0.30015 | 0.61792 |
| V29G | 0.7849 | 0.20412 | 0.1676 | 0.61652 |
| A121V | 0.42199 | 0.81001 | 0.5515 | 0.61446 |
| D49G | 0.39334 | 0.81001 | 0.65108 | 0.61127 |
| V174G | 0.31682 | 0.19853 | 0.29306 | 0.61099 |
| Q139R | 0.21968 | 0.39776 | 0.40319 | 0.60875 |
| L50M | 0.5553 | 0.20516 | 0.11707 | 0.60467 |
| T181A | 0.45756 | 0.08975 | 0.14038 | 0.60333 |
| P24A | 0.52492 | 0.38957 | 0.19405 | 0.60251 |
| T221A | 0.5748 | 0.48141 | 0.3933 | 0.59732 |
| E288G | 0.91255 | 0.33681 | 0.30135 | 0.59468 |
| D145H | 0.65419 | 0.40063 | 0.42964 | 0.59434 |
| D145Y | 0.72154 | 0.41509 | 0.41729 | 0.59399 |
| E218Q | 0.38185 | 0.08975 | 0.34476 | 0.59319 |
| E58K | 0.539 | 0.81001 | 0.52151 | 0.59279 |
| E288K | 0.91255 | 0.35257 | 0.42853 | 0.59224 |
| N131H | 0.24857 | 0.81001 | 0.18617 | 0.59164 |
| L106F | 0.59928 | 0.23188 | 0.10071 | 0.59064 |
| T20A | 0.63226 | 0.58761 | 0.40822 | 0.58974 |
| V174F | 0.42199 | 0.08975 | 0.19268 | 0.58442 |
| N219I | 0.37536 | 0.43096 | 0.54591 | 0.5836 |
| I65M | 0.539 | 0.27357 | 0.25504 | 0.58319 |
| H172Q | 0.33753 | 0.30657 | 0.16442 | 0.57998 |
| E31G | 0.65419 | 0.19072 | 0.35209 | 0.57731 |
| I206T | 0.21884 | 0.2802 | 0.23357 | 0.5753 |
| E217Q | 0.14303 | 0.08975 | 0.15236 | 0.5746 |
| I141M | 0.56456 | 0.33601 | 0.35108 | 0.57248 |
| E289D | 0.91255 | 0.31514 | 0.38662 | 0.57237 |
| D215Y | 0.46129 | 0.08975 | 0.22437 | 0.57055 |
| E31K | 0.56456 | 0.20864 | 0.32541 | 0.57044 |
| S279L | 0.72154 | 0.32987 | 0.22504 | 0.56847 |
| L16R | 0.91255 | 0.33651 | 0.71196 | 0.56714 |
| V174A | 0.14159 | 0.08975 | 0.28506 | 0.56698 |
| G110E | 0.91255 | 0.81001 | 0.75973 | 0.56675 |
| G110R | 0.91255 | 0.81001 | 0.75959 | 0.56675 |
| D26N | 0.59928 | 0.81001 | 0.35268 | 0.56537 |
| E217K | 0.09182 | 0.08975 | 0.1483 | 0.56161 |
| D171G | 0.36709 | 0.33535 | 0.3359 | 0.55583 |
| V174D | 0.23905 | 0.19486 | 0.25602 | 0.5545 |
| K162N | 0.21884 | 0.18198 | 0.18372 | 0.55409 |
| N131S | 0.39097 | 0.81001 | 0.2579 | 0.55292 |
| G177V | 0.15665 | 0.21894 | 0.28952 | 0.55139 |
| E187D | 0.1361 | 0.08975 | 0.16852 | 0.55127 |
| V23I | 0.32296 | 0.81001 | 0.47677 | 0.54968 |
| Q66E | 0.13744 | 0.44734 | 0.24965 | 0.54748 |
| M59I | 0.37536 | 0.81001 | 0.25374 | 0.54695 |
| P146L | 0.72154 | 0.49076 | 0.32645 | 0.54532 |
| I38M | 0.00964 | 0.81001 | 0.19299 | 0.54368 |
| H78R | 0.63226 | 0.81001 | 0.53096 | 0.54325 |
| R125Q | 0.11623 | 0.81001 | 0.37061 | 0.54003 |
| D145E | 0.61437 | 0.31128 | 0.34983 | 0.53726 |
| N35D | 0.59928 | 0.81001 | 0.75557 | 0.53695 |
| M1I | 0.91255 | 0.27177 | 0.65428 | 0.53448 |
| N138K | 0.43085 | 0.38345 | 0.40951 | 0.53448 |
| H151R | 0.12714 | 0.22212 | 0.19243 | 0.53371 |
| V76F | 0.33418 | 0.08975 | 0.14888 | 0.53211 |
| S79N | 0.39575 | 0.08975 | 0.19229 | 0.52726 |
| I226L | 0.21801 | 0.47961 | 0.45451 | 0.52372 |
| G177D | 0.1292 | 0.08975 | 0.27107 | 0.52263 |
| Q173E | 0.23905 | 0.21372 | 0.18337 | 0.51885 |
| K189Q | 0.13392 | 0.08975 | 0.25465 | 0.51505 |
| R125L | 0.18184 | 0.81001 | 0.39104 | 0.50783 |
| N131I | 0.4691 | 0.81001 | 0.25526 | 0.50732 |
| A85V | 0.21718 | 0.08975 | 0.26454 | 0.50567 |
| D73N | 0.29688 | 0.81001 | 0.53015 | 0.50234 |
| G177S | 0.06482 | 0.08975 | 0.28068 | 0.50166 |
| E205K | 0.22312 | 0.21765 | 0.3202 | 0.5015 |
| R82I | 0.24857 | 0.08975 | 0.17431 | 0.50143 |
| P43Q | 0.37966 | 0.81001 | 0.56022 | 0.5009 |
| V68A | 0.21144 | 0.25703 | 0.27508 | 0.50052 |
| K46R | 0.08865 | 0.08975 | 0.10069 | 0.49919 |
| T203S | 0.27544 | 0.20482 | 0.23582 | 0.49722 |
| S80R | 0.29029 | 0.41361 | 0.33018 | 0.49561 |
| R212K | 0.02625 | 0.08975 | 0.22602 | 0.49497 |
| N183H | 0.00964 | 0.08975 | 0.15061 | 0.49147 |
| N131K | 0.00964 | 0.81001 | 0.17099 | 0.49113 |
| T290A | 0.91255 | 0.29346 | 0.45769 | 0.48775 |
| V29M | 0.21224 | 0.08975 | 0.16666 | 0.48677 |
| I126L | 0.11844 | 0.81001 | 0.70654 | 0.48632 |
| N183D | 0.3775 | 0.08975 | 0.14575 | 0.48549 |
| E45Q | 0.08969 | 0.08975 | 0.15228 | 0.48512 |
| E45K | 0.04482 | 0.08975 | 0.11567 | 0.48465 |
| R140I | 0.5748 | 0.46125 | 0.37289 | 0.48455 |
| F211I | 0.91255 | 0.43893 | 0.49602 | 0.48455 |
| A98V | 0.07259 | 0.08975 | 0.06806 | 0.48418 |
| E188D | 0.16198 | 0.08975 | 0.26504 | 0.48155 |
| I226M | 0.59928 | 0.40113 | 0.34182 | 0.48074 |
| R2G | 0.91255 | 0.29103 | 0.33913 | 0.47942 |
| K178E | 0.17239 | 0.08975 | 0.27821 | 0.4776 |
| Q107K | 0.05106 | 0.08975 | 0.08973 | 0.47717 |
| H240P | 0.63226 | 0.43196 | 0.30041 | 0.47572 |
| H220Y | 0.03227 | 0.24179 | 0.27594 | 0.47561 |
| V68E | 0.42783 | 0.26172 | 0.25397 | 0.47085 |
| H220D | 0.20078 | 0.25492 | 0.2888 | 0.46977 |
| D215H | 0.24468 | 0.08975 | 0.22254 | 0.46854 |
| T180I | 0.32296 | 0.08975 | 0.10672 | 0.46719 |
| L244P | 0.36709 | 0.50806 | 0.44436 | 0.46556 |
| A51S | 0.03402 | 0.08975 | 0.0412 | 0.46338 |
| D61E | 0.07545 | 0.08975 | 0.06754 | 0.46325 |
| P216H | 0.06648 | 0.08975 | 0.136 | 0.46311 |
| H69N | 0.00964 | 0.08975 | 0.04132 | 0.46285 |
| D103N | 0.08762 | 0.81001 | 0.18554 | 0.46233 |
| R186G | 0.15406 | 0.08975 | 0.10846 | 0.46142 |
| N138T | 0.40319 | 0.34356 | 0.3585 | 0.46011 |
| K189M | 0.36709 | 0.08975 | 0.27909 | 0.45891 |
| D215N | 0.08609 | 0.08975 | 0.21129 | 0.45837 |
| P216S | 0.09543 | 0.08975 | 0.12949 | 0.45783 |
| K189E | 0.17701 | 0.08975 | 0.2617 | 0.45635 |
| A98T | 0.35349 | 0.08975 | 0.11408 | 0.45513 |
| S169N | 0.00964 | 0.22571 | 0.17674 | 0.4521 |
| H151L | 0.05046 | 0.2384 | 0.21265 | 0.45112 |
| R186K | 0.00964 | 0.08975 | 0.17818 | 0.45112 |
| V147A | 0.03471 | 0.24617 | 0.20075 | 0.45084 |
| R213K | 0.24955 | 0.08975 | 0.26111 | 0.44829 |
| K129N | 0.68238 | 0.81001 | 0.44422 | 0.44702 |
| E188Q | 0.47745 | 0.08975 | 0.33403 | 0.44702 |
| P216L | 0.03903 | 0.08975 | 0.09415 | 0.44673 |
| N219T | 0.22138 | 0.40826 | 0.55324 | 0.44415 |
| Q91E | 0.23271 | 0.81001 | 0.4067 | 0.44212 |
| R113S | 0.13566 | 0.81001 | 0.25909 | 0.44168 |
| K185E | 0.1909 | 0.08975 | 0.1239 | 0.44051 |
| L16M | 0.72154 | 0.27094 | 0.48981 | 0.44051 |
| F211L | 0.91255 | 0.44098 | 0.48091 | 0.43859 |
| N96S | 0.52492 | 0.08975 | 0.19685 | 0.43103 |
| R186T | 0.08188 | 0.08975 | 0.17635 | 0.42584 |
| E188V | 0.72154 | 0.08975 | 0.31269 | 0.42505 |
| E188K | 0.44694 | 0.08975 | 0.31261 | 0.42473 |
| H69Y | 0.47745 | 0.08975 | 0.10146 | 0.42314 |
| L50V | 0.50132 | 0.20516 | 0.14703 | 0.42234 |
| M36V | 0.00964 | 0.81001 | 0.13118 | 0.41944 |
| T181I | 0.14996 | 0.08975 | 0.07843 | 0.41927 |
| A5D | 0.91255 | 0.08975 | 0.05064 | 0.41927 |
| L88F | 0.53172 | 0.08975 | 0.2661 | 0.41863 |
| E31A | 0.56456 | 0.19072 | 0.3626 | 0.41764 |
| R2M | 0.91255 | 0.28273 | 0.40012 | 0.41549 |
| V174L | 0.2708 | 0.08975 | 0.20116 | 0.41498 |
| A5V | 0.91255 | 0.08975 | 0.052 | 0.4138 |
| K162Q | 0.0866 | 0.08975 | 0.14569 | 0.41211 |
| E288D | 0.91255 | 0.26787 | 0.32735 | 0.4066 |
| K162R | 0.03555 | 0.08975 | 0.21244 | 0.4052 |
| M115T | 0.68238 | 0.81001 | 0.46236 | 0.39879 |
| K75T | 0.35165 | 0.08975 | 0.14438 | 0.39879 |
| K185N | 0.19023 | 0.08975 | 0.15969 | 0.39843 |
| G264V | 0.20002 | 0.37168 | 0.09026 | 0.39735 |
| R2W | 0.91255 | 0.31263 | 0.40168 | 0.39644 |
| Q275R | 0.00964 | 0.08975 | 0.03784 | 0.39255 |
| A85T | 0.43085 | 0.08975 | 0.28314 | 0.39218 |
| L142W | 0.7849 | 0.20745 | 0.27611 | 0.39106 |
| L94Q | 0.40832 | 0.08975 | 0.11956 | 0.38664 |
| T203I | 0.08408 | 0.22782 | 0.2067 | 0.38527 |
| R82K | 0.24758 | 0.08975 | 0.12946 | 0.38488 |
| K89N | 0.36912 | 0.35134 | 0.28117 | 0.3833 |
| S79G | 0.42783 | 0.08975 | 0.19278 | 0.38092 |
| Q83H | 0.23813 | 0.08975 | 0.18148 | 0.37448 |
| P227S | 0.43708 | 0.4991 | 0.3454 | 0.3722 |
| V128L | 0.00964 | 0.81001 | 0.2444 | 0.3722 |
| N63Y | 0.45756 | 0.08975 | 0.09264 | 0.3703 |
| H69L | 0.38863 | 0.08975 | 0.23102 | 0.36946 |
| E58G | 0.38863 | 0.81001 | 0.56435 | 0.36819 |
| D103H | 0.40068 | 0.81001 | 0.25056 | 0.36235 |
| I64V | 0.05436 | 0.31138 | 0.20017 | 0.36082 |
| P43S | 0.37118 | 0.81001 | 0.55527 | 0.35705 |
| H14Y | 0.41637 | 0.22916 | 0.32832 | 0.35571 |
| T127A | 0.40319 | 0.81001 | 0.40861 | 0.35548 |
| L142F | 0.44694 | 0.19072 | 0.26892 | 0.35458 |
| D268H | 0.53172 | 0.41945 | 0.44247 | 0.35322 |
| F4S | 0.91255 | 0.08975 | 0.11753 | 0.35299 |
| I3T | 0.91255 | 0.26804 | 0.28311 | 0.35139 |
| H14R | 0.30097 | 0.25813 | 0.41771 | 0.35071 |
| S283T | 0.17353 | 0.08975 | 0.29893 | 0.35071 |
| V242A | 0.36709 | 0.08975 | 0.0693 | 0.3484 |
| G252D | 0.20002 | 0.08975 | 0.12642 | 0.34724 |
| V30A | 0.22053 | 0.18198 | 0.24125 | 0.34678 |
| R140T | 0.34982 | 0.30029 | 0.37836 | 0.34069 |
| I199V | 0.18184 | 0.22015 | 0.14676 | 0.33975 |
| H240L | 0.37536 | 0.43243 | 0.29856 | 0.33975 |
| R238T | 0.5748 | 0.41754 | 0.46267 | 0.33856 |
| I243T | 0.28026 | 0.08975 | 0.14085 | 0.33832 |
| M36T | 0.59928 | 0.81001 | 0.6727 | 0.33687 |
| T285A | 0.5553 | 0.08975 | 0.10902 | 0.33518 |
| A52V | 0.02906 | 0.08975 | 0.11565 | 0.33494 |
| R140K | 0.25873 | 0.26795 | 0.35251 | 0.33371 |
| G264E | 0.16738 | 0.08975 | 0.22123 | 0.33126 |
| Q47R | 0.10874 | 0.08975 | 0.09244 | 0.32978 |
| L249V | 0.26965 | 0.08975 | 0.1777 | 0.32928 |
| Q47E | 0.23997 | 0.08975 | 0.13134 | 0.32903 |
| Q282R | 0.08433 | 0.08975 | 0.20807 | 0.32878 |
| M10I | 0.25055 | 0.08975 | 0.06181 | 0.32727 |
| E237K | 0.05992 | 0.08975 | 0.04186 | 0.32576 |
| I8V | 0.91255 | 0.08975 | 0.10592 | 0.32525 |
| P230S | 0.2556 | 0.08975 | 0.07296 | 0.32475 |
| G264W | 0.53172 | 0.08975 | 0.12217 | 0.3227 |
| T22S | 0.25355 | 0.51308 | 0.34083 | 0.32218 |
| E71K | 0.02613 | 0.19925 | 0.0591 | 0.32141 |
| V44A | 0.49117 | 0.81001 | 0.6152 | 0.3188 |
| M115L | 0.00964 | 0.81001 | 0.23369 | 0.31749 |
| V130L | 0.539 | 0.81001 | 0.72164 | 0.31749 |
| P227A | 0.14064 | 0.50225 | 0.35188 | 0.31617 |
| R262I | 0.72154 | 0.20667 | 0.18867 | 0.31459 |
| L248S | 0.59928 | 0.08975 | 0.15422 | 0.31326 |
| L251F | 0.1909 | 0.08975 | 0.10923 | 0.31245 |
| L255Q | 0.539 | 0.08975 | 0.21105 | 0.31028 |
| S149Y | 0.43708 | 0.31492 | 0.18575 | 0.30646 |
| T203A | 0.02043 | 0.21509 | 0.1003 | 0.30255 |
| T256S | 0.40832 | 0.08975 | 0.11142 | 0.30199 |
| A246V | 0.11659 | 0.08975 | 0.17586 | 0.30143 |
| L229P | 0.17126 | 0.27379 | 0.02246 | 0.30143 |
| D284Y | 0.50132 | 0.81001 | 0.45125 | 0.29284 |
| D145N | 0.34982 | 0.36694 | 0.35477 | 0.29168 |
| C250Y | 0.33254 | 0.08975 | 0.13237 | 0.2908 |
| M59T | 0.28395 | 0.81001 | 0.24244 | 0.28577 |
| I8K | 0.91255 | 0.19072 | 0.15771 | 0.28577 |
| N236H | 0.2852 | 0.08975 | 0.15385 | 0.28126 |
| E237Q | 0.14996 | 0.08975 | 0.10679 | 0.28095 |
| L255R | 0.5553 | 0.08975 | 0.21287 | 0.28004 |
| F259L | 0.04629 | 0.08975 | 0.15967 | 0.27973 |
| I243V | 0.29158 | 0.08975 | 0.15161 | 0.27789 |
| P227T | 0.54683 | 0.4977 | 0.36344 | 0.27603 |
| G252C | 0.22138 | 0.08975 | 0.16616 | 0.27416 |
| Q100R | 0.10945 | 0.81001 | 0.38882 | 0.27292 |
| M10V | 0.18562 | 0.08975 | 0.13622 | 0.27043 |
| A254G | 0.35537 | 0.08975 | 0.15776 | 0.26917 |
| D171N | 0.20759 | 0.30711 | 0.33727 | 0.26854 |
| D49N | 0.03869 | 0.41327 | 0.23534 | 0.26823 |
| I258M | 0.54683 | 0.08975 | 0.18609 | 0.26604 |
| V174I | 0.24372 | 0.08975 | 0.17181 | 0.26541 |
| R260C | 0.20607 | 0.08975 | 0.00785 | 0.2651 |
| G245A | 0.10311 | 0.25222 | 0.2843 | 0.26101 |
| I38T | 0.65419 | 0.81001 | 0.66156 | 0.26038 |
| R238K | 0.29959 | 0.37769 | 0.44176 | 0.26007 |
| K105Q | 0.10447 | 0.81001 | 0.50322 | 0.26007 |
| N135D | 0.65419 | 0.19486 | 0.22053 | 0.26007 |
| E60Q | 0.16198 | 0.81001 | 0.25845 | 0.25943 |
| K263E | 0.45393 | 0.21792 | 0.31151 | 0.25912 |
| R2K | 0.91255 | 0.24169 | 0.27802 | 0.25688 |
| T285S | 0.49613 | 0.08975 | 0.1041 | 0.25688 |
| Q83R | 0.05808 | 0.08975 | 0.06683 | 0.25625 |
| I54L | 0.12592 | 0.08975 | 0.14111 | 0.25497 |
| V242I | 0.28271 | 0.08975 | 0.10331 | 0.25464 |
| D284A | 0.20835 | 0.08975 | 0.22773 | 0.25143 |
| G245E | 0.40573 | 0.26615 | 0.33294 | 0.25045 |
| T285P | 0.61437 | 0.08975 | 0.09477 | 0.24948 |
| M10T | 0.18434 | 0.08975 | 0.07133 | 0.24722 |
| N236D | 0.12187 | 0.08975 | 0.17464 | 0.24592 |
| H69R | 0.30943 | 0.08975 | 0.2277 | 0.24528 |
| N135S | 0.23905 | 0.08975 | 0.06293 | 0.24463 |
| F9L | 0.38185 | 0.08975 | 0.11763 | 0.24301 |
| E228V | 0.49613 | 0.52935 | 0.4191 | 0.24269 |
| Y12F | 0.05992 | 0.08975 | 0.25467 | 0.24139 |
| I141V | 0.20607 | 0.31207 | 0.31755 | 0.24041 |
| G177C | 0.3261 | 0.08975 | 0.3039 | 0.24041 |
| V6I | 0.20078 | 0.08975 | 0.04368 | 0.24008 |
| Q100H | 0.05152 | 0.81001 | 0.40406 | 0.23975 |
| V143A | 0.11478 | 0.18612 | 0.0967 | 0.23616 |
| C250G | 0.24564 | 0.08975 | 0.15762 | 0.23486 |
| T285I | 0.68238 | 0.08975 | 0.11911 | 0.23421 |
| K136E | 0.36912 | 0.25521 | 0.2941 | 0.22802 |
| P230T | 0.195 | 0.08975 | 0.12535 | 0.22736 |
| D61V | 0.72154 | 0.2191 | 0.248 | 0.22704 |
| N183S | 0.14159 | 0.08975 | 0.12917 | 0.22246 |
| L241F | 0.15984 | 0.08975 | 0.16174 | 0.22116 |
| V253I | 0.0831 | 0.08975 | 0.26357 | 0.2205 |
| P24S | 0.27426 | 0.38957 | 0.15688 | 0.21853 |
| M267I | 0.15406 | 0.21167 | 0.16709 | 0.21788 |
| W13C | 0.91255 | 0.21117 | 0.24426 | 0.21299 |
| R260H | 0.07425 | 0.08975 | 0.33842 | 0.21005 |
| F211C | 0.91255 | 0.45899 | 0.49535 | 0.21005 |
| R265K | 0.14595 | 0.08975 | 0.15012 | 0.20908 |
| P235S | 0.09022 | 0.08975 | 0.0905 | 0.20876 |
| I3M | 0.91255 | 0.22111 | 0.1523 | 0.20551 |
| M267T | 0.51248 | 0.08975 | 0.18426 | 0.20454 |
| P133S | 0.05167 | 0.47237 | 0.42281 | 0.2039 |
| E223K | 0.16144 | 0.08975 | 0.1467 | 0.20325 |
| I274M | 0.50132 | 0.08975 | 0.20931 | 0.20227 |
| R113L | 0.07846 | 0.81001 | 0.28813 | 0.20131 |
| K280N | 0.539 | 0.26424 | 0.26297 | 0.19937 |
| D61N | 0.41637 | 0.08975 | 0.17046 | 0.19777 |
| L287V | 0.30097 | 0.08975 | 0.24316 | 0.19618 |
| V76I | 0.10659 | 0.08975 | 0.10975 | 0.19554 |
| N17D | 0.36912 | 0.18612 | 0.21604 | 0.193 |
| S169R | 0.65419 | 0.2802 | 0.485 | 0.19236 |
| L261F | 0.44694 | 0.08975 | 0.13138 | 0.19173 |
| V143I | 0.30097 | 0.20048 | 0.2357 | 0.19141 |
| A232V | 0.14352 | 0.08975 | 0.03905 | 0.18951 |
| T221I | 0.68238 | 0.51968 | 0.4631 | 0.18951 |
| H240Y | 0.68238 | 0.40051 | 0.31208 | 0.18794 |
| V253G | 0.7849 | 0.21986 | 0.21906 | 0.187 |
| D276N | 0.25457 | 0.08975 | 0.19918 | 0.18606 |
| A51D | 0.05214 | 0.08975 | 0.05075 | 0.18513 |
| P235L | 0.2731 | 0.08975 | 0.10541 | 0.18357 |
| P230L | 0.40573 | 0.08975 | 0.09649 | 0.18202 |
| P234S | 0.1155 | 0.08975 | 0.13212 | 0.17832 |
| A51V | 0.11732 | 0.08975 | 0.04838 | 0.17771 |
| A232S | 0.03996 | 0.08975 | 0.12096 | 0.17526 |
| R262G | 0.65419 | 0.18612 | 0.18285 | 0.17434 |
| I258V | 0.14793 | 0.08975 | 0.11341 | 0.17253 |
| F257C | 0.58626 | 0.08975 | 0.18796 | 0.17041 |
| M266I | 0.43708 | 0.08975 | 0.19636 | 0.16921 |
| N96H | 0.51853 | 0.26267 | 0.20285 | 0.16592 |
| K280Q | 0.32929 | 0.23416 | 0.20048 | 0.16562 |
| K75N | 0.26965 | 0.08975 | 0.12792 | 0.16473 |
| L249S | 0.65419 | 0.08975 | 0.26741 | 0.16265 |
| I258F | 0.38185 | 0.08975 | 0.1305 | 0.16148 |
| F257L | 0.1155 | 0.08975 | 0.09555 | 0.16118 |
| R265G | 0.61437 | 0.08975 | 0.24199 | 0.16118 |
| E223Q | 0.25055 | 0.08975 | 0.18369 | 0.16089 |
| G273D | 0.20988 | 0.08975 | 0.09864 | 0.15913 |
| E71Q | 0.19361 | 0.19925 | 0.10475 | 0.15709 |
| I101T | 0.91255 | 0.81001 | 0.6852 | 0.15418 |
| N236I | 0.29823 | 0.08975 | 0.19231 | 0.15418 |
| H233Y | 0.00964 | 0.08975 | 0.04712 | 0.15073 |
| K62N | 0.09401 | 0.24576 | 0.17426 | 0.1496 |
| P234T | 0.14996 | 0.08975 | 0.15023 | 0.1496 |
| A232T | 0.05973 | 0.08975 | 0.10057 | 0.14481 |
| A232G | 0.10909 | 0.08975 | 0.10639 | 0.13954 |
| D276E | 0.44694 | 0.08975 | 0.19794 | 0.13736 |
| P234R | 0.15717 | 0.08975 | 0.13922 | 0.13493 |
| L231M | 0.20912 | 0.08975 | 0.05185 | 0.13493 |
| L244V | 0.37536 | 0.20909 | 0.19166 | 0.13145 |
| L255M | 0.31235 | 0.08975 | 0.13097 | 0.13093 |
| R265I | 0.68238 | 0.24609 | 0.18908 | 0.12803 |
| V111L | 0.13088 | 0.4977 | 0.17954 | 0.12672 |
| R113H | 0.1002 | 0.81001 | 0.28053 | 0.12517 |
| S169C | 0.7849 | 0.27684 | 0.47448 | 0.12439 |
| R86Q | 0.05755 | 0.08975 | 0.03823 | 0.12388 |
| D90E | 0.02465 | 0.81001 | 0.27145 | 0.12362 |
| V269M | 0.20532 | 0.31036 | 0.31572 | 0.12337 |
| V143F | 0.42783 | 0.23459 | 0.2357 | 0.11981 |
| A85S | 0.5748 | 0.08975 | 0.32068 | 0.11855 |
| D61H | 0.65419 | 0.08975 | 0.23128 | 0.11681 |
| L244M | 0.35537 | 0.20694 | 0.19024 | 0.11656 |
| C272Y | 0.31088 | 0.35168 | 0.38027 | 0.11607 |
| D276H | 0.50676 | 0.08975 | 0.2089 | 0.11118 |
| D276Y | 0.56456 | 0.08975 | 0.22053 | 0.11118 |
| R265T | 0.59928 | 0.08975 | 0.20367 | 0.11118 |
| A246D | 0.26965 | 0.08975 | 0.22397 | 0.10997 |
| T239S | 0.23271 | 0.28388 | 0.29521 | 0.10902 |
| F7L | 0.08359 | 0.08975 | 0.1031 | 0.10246 |
| G245V | 0.33418 | 0.46353 | 0.32846 | 0.09723 |
| A232P | 0.15561 | 0.08975 | 0.13052 | 0.09458 |
| L231V | 0.07521 | 0.08975 | 0.04523 | 0.09413 |
| G252S | 0.10412 | 0.08975 | 0.0917 | 0.09282 |
| E223V | 0.23631 | 0.08975 | 0.14776 | 0.09065 |
| R262K | 0.12305 | 0.08975 | 0.08508 | 0.08083 |
| G264R | 0.2708 | 0.08975 | 0.05302 | 0.07701 |
| F207L | 0.63226 | 0.43474 | 0.49787 | 0.07349 |
| R113C | 0.25768 | 0.81001 | 0.20826 | 0.0731 |
| K271N | 0.34269 | 0.36339 | 0.31517 | 0.07233 |
| M36I | 0.54683 | 0.81001 | 0.39734 | 0.07137 |
| T148A | 0.48642 | 0.34888 | 0.22288 | 0.06965 |
| Y28S | 0.61437 | 0.20581 | 0.32755 | 0.06852 |
| D268N | 0.27544 | 0.3866 | 0.40674 | 0.06498 |
| D103Y | 0.5553 | 0.81001 | 0.243 | 0.0648 |
| Y160H | 0.91255 | 0.40624 | 0.41461 | 0.06407 |
| C272R | 0.50132 | 0.53665 | 0.32021 | 0.05735 |
| D284N | 0.20154 | 0.81001 | 0.47931 | 0.05549 |
| E228G | 0.33418 | 0.51308 | 0.39979 | 0.05449 |
| N96Y | 0.58626 | 0.26267 | 0.2356 | 0.04869 |
| L53I | 0.2345 | 0.23778 | 0.13427 | 0.0364 |
| D268G | 0.40573 | 0.43662 | 0.49607 | 0.03517 |
| K281N | 0.26192 | 0.08975 | 0.09815 | 0.02832 |
| D61Y | 0.72154 | 0.08975 | 0.22502 | 0.02733 |
| L48S | 0.7849 | 0.81001 | 0.56721 | 0.01744 |
| A157T | 0.68238 | 0.4168 | 0.38343 | 0.01689 |
| L53R | 0.7849 | 0.81001 | 0.35684 | 0.01458 |
| W167C | 0.91255 | 0.81001 | 0.57613 | 0.0099 |
| A222G | 0.45039 | 0.4486 | 0.48385 | 0.00961 |
| D122E | 0.91255 | 0.81001 | 0.51077 | 0.00778 |
| V55F | 0.72154 | 0.81001 | 0.68928 | 0.00496 |
| T37A | 0.65419 | 0.81001 | 0.75557 | 0.00496 |
| K124N | 0.91255 | 0.81001 | 0.5607 | 0.00464 |
| T37K | 0.7849 | 0.81001 | 0.62191 | 0.00359 |
| Y123H | 0.91255 | 0.81001 | 0.45765 | 0.00331 |
| A222V | 0.56456 | 0.45092 | 0.45407 | 0.00294 |
| G95E | 0.68238 | 0.81001 | 0.74713 | 0.0029 |
| P161L | 0.91255 | 0.81001 | 0.49773 | 0.00275 |
| P161S | 0.91255 | 0.53665 | 0.49944 | 0.00275 |
| L53P | 0.7849 | 0.81001 | 0.34164 | 0.0027 |
| G70R | 0.68238 | 0.81001 | 0.78218 | 0.00228 |
| D122N | 0.91255 | 0.81001 | 0.76697 | 0.00211 |
| I126F | 0.65419 | 0.81001 | 0.69353 | 0.00206 |
| D122Y | 0.91255 | 0.81001 | 0.79327 | 0.00095 |
| I126S | 0.7849 | 0.81001 | 0.64709 | 0.00093 |
| G95R | 0.42783 | 0.81001 | 0.6625 | 0.00033 |
| G70E | 0.63226 | 0.81001 | 0.78218 | 0.00022 |
| G70A | 0.63226 | 0.81001 | 0.81754 | 0.00022 |

### DISCUSSION

The results described in this Example provide a study of a large cohort of 1,081 clinically advanced malignancies with CD274 non-amplification SV mutations, including 213 samples with concurrent PD-L1 protein expression levels. The overall prevalence of CD274 non-amplification SV mutations across tumor types was low (0.3%, 1,081/314,631), and most of the SV mutations found were missense substitutions, with rarer nonsense and indel alterations. Lastly, the prevalence of CD274 SV mutations was higher in patients with MSI-high associated endometrial and UV light exposed cutaneous cancers.

While most of the non-truncating SV CD274 mutations had low to no tumor cell expression of PD-L1, the expression levels of PD-L1 differed among the various CD274 SV mutation categories. Interestingly, of the eleven R260H cases concurrently tested with PD-L1 IHC, most showed little to no PD-L1 expression, suggesting that patients with R260H mutations might not be treated with ICPI if only tested with PD-L1 IHC. This contrasted with the five E237K cases, where most cases had some PD-L1 expression, suggesting that the E237K has little to no effect on PD-L1 protein expression. Most other variants were only observed in a single sample (with or without concurrent PD-L1 expression data) due the rarity of the mutations.

Samples with clonal truncating mutations had a lower level of PD-L1 expression when compared with cases without a CD274 mutation and to samples with sub-clonal truncating mutations. These data suggest that when a clonal truncating event is observed in CD274, PD-L1 expression is inhibited, but in the setting of a sub-clonal truncation, clinical assessment of sample-level PD-L1 expression is often not affected. Clonal truncating variants can act as resistance biomarkers for ICPI due the lack of PD-L1 protein present on the tumor cells as exemplified by the PD-L1 IHC expression data described herein. With decreased or no ligand for the PD-L1/PD-1 inhibitors to bind to, the efficacy of ICPI would likely be diminished.

CD274 missense mutations could mediate resistance to ICPI due to potential steric or affinity-altering interferences in the binding of the PD-L1 ligand to the PD-1 receptor, similar to a resistance mechanism described for ROS1 (Huang et al., JTO Clinical and Research Reports (2020) 100100; Huang et al., Int J Cancer (2021) 148(7):1778-1788). A slightly lower level of PD-L1 IHC staining in the cases with CD274 missense mutations was observed, when compared to cases without CD274 mutations. In addition, a significantly lower PD-L1 IHC staining in the clonal missense mutations was observed, when compared to the sub-clonal missense mutations. This could be due to the lower rates of PD-L1 antibody binding (from the IHC assay) to the PD-L1 ligand on the tumor cells instead of actual lower PD-L1 protein expression. No correlation between the predicted functional status of missense mutations and PD-L1 IHC expression was observed. The functionality prediction algorithms do not formally consider the steric or binding affinity interferences that can result from missense mutations into the functionality assessment. Taken together, the results described herein suggest that CD274 mutations are a potential mechanism for resistance of cancers to ICPI.

In conclusion, this Example describes the landscape of CD274 mutations in a large pan-cancer cohort that can be used for examining CD274 mutations as potential resistance biomarkers for ICPI. Furthermore, novel data on the correlation of CD274 mutations and PD-L1 protein expression is presented, which provides important data on the functionality of these mutations.

1-11. (canceled)

12. A method of treating or delaying progression of cancer, comprising:
(a) acquiring knowledge of one or more mutations in a CD274 gene in a sample from an individual; and
(b) responsive to said knowledge, administering to the individual an effective amount of a treatment that comprises an anti-cancer therapy.

13. A method of treating or delaying progression of cancer, comprising, responsive to acquiring knowledge of one or more mutations in a CD274 gene in a sample from an individual, administering to the individual an effective amount of a treatment that comprises an anti-cancer therapy.

14-16. (canceled)

17. A method of treating or delaying progression of cancer, comprising:
(a) detecting one or more mutations in a CD274 gene in a sample from an individual; and
(b) administering to the individual an effective amount of a treatment that comprises an anti-cancer therapy.

18-24. (canceled)

25. The method of claim 17, wherein the anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

26-30. (canceled)

31. The method of claim 17, wherein the one or more mutations in a CD274 gene comprise one or more mutations listed in Tables 1-4 and 6.

32. The method of claim 31, wherein the one or more mutations in a CD274 gene further comprise:
(a) a CD274 gene deletion, wherein the CD274 gene is a deletion of a portion of the CD274 gene; and/or
(b) a CD274 genomic rearrangement or a CD274 gene fusion.

33-36. (canceled)

37. The method of claim 31, wherein the one or more mutations in a CD274 gene further comprise a CD274 gene amplification.

38. The method of claim 17, wherein the one or more mutations in a CD274 gene result in:
(a) low expression of a PD-L1 protein in the cancer,
(b) no expression of a PD-L1 protein in the cancer, or
(c) high expression of a PD-L1 protein in the cancer.

39-41. (canceled)

42. The method of claim 38, wherein the one or more mutations in a CD274 gene comprise one or more mutations listed in Table 2 or Table 3.

43-45. (canceled)

46. The method of claim 38, wherein the one or more mutations in a CD274 gene comprise one or more mutations listed in Table 4.

47-48. (canceled)

49. The method of claim 17, wherein: (a) the one or more mutations in a CD274 gene reduce the interaction between a PD-L1 polypeptide encoded by the CD274 gene and a PD-1 receptor; and/or (b) the one or more mutations in a CD274 gene reduce the activity of a PD-L1 polypeptide encoded by the CD274 gene.

50. The method of claim 17, wherein the one or more mutations in a CD274 gene result in an immune checkpoint inhibitor resistant cancer.

51. The method of claim 17, wherein the anti-cancer therapy is a therapy other than an immune checkpoint inhibitor.

52-55. (canceled)

56. The method of claim 17, further comprising selectively enriching for one or more nucleic acids comprising nucleotide sequences that comprise the one or more mutations in the CD274 gene; wherein the selectively enriching produces an enriched sample.

57-59. (canceled)

60. The method of claim 17, wherein the cancer is a carcinoma, a sarcoma, a lymphoma, a leukemia, a myeloma, a germ cell cancer, or a blastoma.

61-62. (canceled)

63. The method of claim 17, wherein the cancer is a cancer listed in Table 5 or Table 6.

64. (canceled)

65. The method of claim 17, wherein the cancer is a skin cancer.

66-76. (canceled)

77. The method of claim 17, wherein the cancer is a non-serous endometrial adenocarcinoma.

78-79. (canceled)

80. The method of claim 17, wherein the cancer is a cancer comprising a CD274 mutation as listed in Table 6.

81-82. (canceled)

83. The method of claim 17, wherein the sample:
(a) is a formalin-fixed paraffin-embedded (FFPE) sample,
(b) comprises fluid, cells, or tissue;
(c) comprises a tumor biopsy or a circulating tumor cell; or
(d) is a nucleic acid sample.

84-132. (canceled)


SEQUENCE LISTING (2 sequences)

SEQ ID NO: 1 - DNA, 3634 bp, Homo sapiens
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 2 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET